(19) United States
(12) Patent Application Publication
(10) Pub. No.: US 2024/0168030 A1
(43) Pub. Date: May 23, 2024

(54) DIRECT FLUORESCENT GLYCAN LABELING

(71) Applicant: BIO-TECHNE CORPORATION, Minneapolis, MN (US)

(72) Inventors: Zhengliang Wu, Edina, MN (US); Mark Russell Whittaker, Saint Paul, MN (US); Anthony D. Person, Blaine, MN (US); Vassilios Kalabokis, Fridley, MN (US); James Michael Ertelt, Watertown, MN (US)

(21) Appl. No.: 17/773,282

(22) PCT Filed: Oct. 30, 2020
(86) PCT No.: PCT/US2020/058282

§ 371 Date: Apr. 29, 2022

Related U.S. Application Data
(60) Provisional application No. 62/929,318, filed on Nov. 1, 2019.
(60) Provisional application No. 62/993,920, filed on Mar. 24, 2020.

(51) Int. Cl. G01N 33/58

(52) U.S. Cl. CPC G01N 33/582

(57) ABSTRACT

This disclosure describes compositions including fluorophore-conjugated sialic acids and fluorophore-conjugated fucose and methods of making and using those compositions. A fluorophore-conjugated sialic acid may include a cytidine monophosphate activated fluorophore-conjugated sialic acids (CMP-f-SA); a fluorophore-conjugated fucose may include a guanosine 5′-diphosphate activated fluorophore-conjugated fucose (GDP-f-Fuc). Methods of using the compositions include enzymatic incorporation of a fluorophore-conjugated sialic acid or a fluorophore-conjugated fucose or both to label and detect N- and O-glycans on glycoproteins. The compositions and methods may allow for the detection of specific glycans.

### CONTINUING APPLICATION DATA

This application claims the benefit of U.S. Provisional Application Ser. No. 62/929,318, filed Nov. 1, 2019, and U.S. Provisional Application Ser. No. 62/993,920, filed Mar. 24, 2020, each of which is incorporated by reference herein in its entirety.

### BACKGROUND

Glycosylation is the reaction in which a carbohydrate is attached to a hydroxyl or other functional group of another molecule. In particular, in biology, glycosylation refers to the enzymatic process that attaches glycans to proteins or other organic molecules.

A “glycan,” also referred to as a polysaccharide, is a compound that includes monosaccharides linked in various combinations and linkages, and featuring diverse and asymmetric types of branching. In some cases, “glycan” may also be used to refer to the carbohydrate portion of a glycoconjugate, including, for example, the carbohydrate portion of a glycoprotein, the carbohydrate portion of a glycolipid, or the carbohydrate portion of a proteoglycan.

Glycans are commonly found on cell membrane and secreted proteins, the result of the post-translational modification of most proteins expressed by mammalian cells. Glycans are frequently terminated with sialic acid, a negatively charged monosaccharide, or fucose, a deoxy hexosaccharide.

Due to the important role of glycosylation and glycans, in particular in antibody function, understanding how glycans are regulated is increasingly important. For example, sialic acids and fucose are essential constituents of various glycan epitopes that are recognized by lectins and antibodies and involved in important biological roles.

Direct detection of glycans on intact biomolecules continues to be a challenge, however.

### Methods of Making a Fluorophore-Conjugated Sialic Acid

The fluorophore-conjugated sialic acid (for example, CMP-f-SA) may be prepared by any suitable method. In some embodiments, an activated fluorophore-conjugated sialic acid may be prepared via copper (I)-catalyzed azide-alkyne cycloaddition. For example, incubating a CMP-Azido-Sialic acid (CMP-N₃-SA) and an alkyne-conjugated fluorophore results in conjugation between the components via copper (I)-catalyzed azide-alkyne cycloaddition.

In some embodiments, a method of preparing an activated fluorophore-conjugated sialic acid may further include purifying and/or concentrating the activated fluorophore-conjugated sialic acid.

In some embodiments, CMP-f-SA may be prepared as described in Example 1.

### SUMMARY OF THE INVENTION

This disclosure describes fluorophore-conjugated sialic acids and fluorophore-conjugated fucose and methods that include enzymatic incorporation of a fluorophore-conjugated sialic acid or a fluorophore-conjugated fucose or both to label and detect N- and O-glycans on glycoproteins. These compositions and methods allow for the detection of specific glycans without the laborious gel blotting and chemiluminescence reactions used in Western blotting and the detection of a glycan in its native state.

In one aspect, this disclosure describes a composition that includes fluorophore-conjugated sialic acid or a fluorophore-conjugated fucose or both a fluorophore-conjugated sialic acid and a fluorophore-conjugated fucose

In another aspect, this disclosure describes a method of making a fluorophore-conjugated sialic acid or a fluorophore-conjugated fucose. The method of making a fluorophore-conjugated sialic acid includes incubating a CMP-Azido-Sialic acid (CMP-N₃-SA) and an alkyne-conjugated fluorophore. In some embodiments, the method further includes forming cytidine monophosphate activated fluorophore-conjugated sialic acid (CMP-f-SA). The method of making a fluorophore-conjugated fucose includes incubating a GDP-Azido-Fucose (GDP-N₃-Fucose) and an alkyne-conjugated fluorophore. In some embodiments, the method further includes forming guanosine diphosphate activated fluorophore-conjugated fucose (GDP-f-Fuc).

In a further aspect, this disclosure describes a method that includes using a fluorophore-conjugated sialic acid or a fluorophore-conjugated fucose or both. In some embodiments, the method includes attaching the fluorophore-conjugated sialic acid or the fluorophore-conjugated fucose or both to a glycan. In some embodiments, the method the glycan is present on a target protein.

In some embodiments, the method includes mixing the glycan or a target protein comprising the glycan, a fluorophore-conjugated sugar comprising the fluorophore-conjugated sialic acid or the fluorophore-conjugated fucose or both, and an enzyme comprising a sialyltransferase or a fucosyltransferase or both. The fluorophore-conjugated sugar is attached to the glycan to form a labeled glycan or a labeled target protein.

In some embodiments, when a labeled target protein is formed, the method may include separating components of a mixture including the labeled target protein. This method may permit analysis and detection of a glycan in its native state—that is, without cleaving glycans from a glycoprotein or glycolipid—providing valuable information about the whole glycan structure.

In some embodiments, when a labeled target protein is formed, the method may include cleaving the labeled glycan from the labeled target protein to form a freed labeled glycan. This method may permit comparison of the freed labeled glycan with a glycan standard or a glycan ladder, allowing for identification of the freed labeled glycan.

In yet another aspect, this disclosure describes a glycan ladder that includes two or more labeled (for example, fluorophore-conjugated) glycans.

As used herein, the terms “incubating” and “incubated” and the like refer to a process that includes exposing the contents of a mixture to the other components of the mixture while maintaining a state of controlled conditions (including, for example, a particular temperature) over a period of time; the term does not imply any particular time or temperature requirement, unless otherwise specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity but include the general class of which a specific example may be used for illustration. The terms “a,” “an,” and “the” are used interchangeably with the term “at least one.”

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Also herein, the recitations of numerical ranges by endpoints include all numbers subsumed within that range (for example, 1 to 5 includes 1, 1.5, 2, 2.75, 3, 3.80, 4, 5, etc.). Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### BRIEF DESCRIPTION OF THE FIGURES

FIG. 1A-FIG. 1C show an exemplary method of Direct Fluorescent Glycan Labeling (DFGL) (FIG. 1A) and a labeling reaction on N-glycans (FIG. 1B) and O-glycans (FIG. 1C). In each case, existing terminal sialic acids may be removed by neuraminidase treatment and replaced with fluorophore-conjugated sialic acids. Neu, neuraminidase; STs, sialyltransferases; CMP, cytidine monophosphate. Monosaccharide symbols follow the SNFG (Symbol Nomenclature for Glycans) system (Varki et al. Glycobiology 25, 1323-1324 (2015)).

FIG. 2A-FIG. 2B show direct fluorescent glycan labeling of fetal bovine fetuin and asialofetuin. All labeled samples were separated on SDS-PAGE and imaged by both trichloroethanol (TCE) staining (top panels) and fluorescent imaging (lower panels). FIG. 2A. Fetal bovine fetuin (F) and asialofetuin (AF) were labeled by α-2,3-sialyltransferase 1 (ST3Gal1 or 31), α-2,6-sialyltransferase 1 (ST6Gal1 or 61) and α-N-acetylgalactosaminide α-2,6-sialyltransferase 4 (ST6GalNAc4 or A4) with cytidine monophosphate activated Alexa-Fluor®488-conjugated sialic acid (CMP-AF488-SA) (AF488), cytidine monophosphate activated Cy5-conjugated sialic acid (CMP-Cy5-SA) (Cy5), and cytidine monophosphate activated Alexa-Fluor® 555-conjugated sialic acid (CMP-AF555-SA) (AF555). FIG. 2B. Labeling of fetuin and asialofetuin samples by α-2,3-sialyltransferase 2 (ST3Gal2), α-N-acetylgalactosaminide α-2,6-sialyltransferase 1 (ST6GalNAc1), α-N-acetylgalactosaminide α-2,6-sialyltransferase 2 (ST6GalNAc2) and α-2,3-sialyltransferase 4 (ST3Gal4) with AF555, Cy5, and AF488. Asialofetuin in FIG. 2A was purchased from Sigma Aldrich (St. Louis, MO). Asialofetuin in FIG. 2B was generated from fetuin by addition of C. perfringens neuraminidase (Neu) to the labeling reactions. ST6GalNAc1 exhibited self-labeling (indicated with arrow in FIG. 2B). The amount of protein (2.5 micrograms (m)) was loaded into each lane; however, due to the presence of multiple benzene rings, Alexa Fluor®-labeled samples exhibited significantly increased band intensities in TCE images. M, molecular marker.

FIG. 3A-FIG. 3B show differential labeling of O-glycans and N-glycans on recombinant mucins and integrins. O-glycans and N-glycans on recombinant mucins and integrins were labeled by ST3Gal1, ST6Gal1, ST6GalNAc1 and ST3Gal4 with Cy5. All samples were pretreated with recombinant C. perfringens neuraminidase to remove preexisting sialic acids. The labeling reactions were separated on SDS-PAGE and imaged by silver staining (FIG. 3A) and fluorescent imager (FIG. 3B). The same amount of protein (2.5 μg) was loaded into each lane. M, Western molecular marker (Bio-Rad Laboratories, Hercules, CA). MUC1 was found to exclusively contain O-glycans, as it was only labeled by ST3Gal1 and ST6GalNAc1; MUC16 was found to contain both N-and 0-glycans, as it was labeled strongly by all four enzymes. All integrins were found to contain N-glycans but not O-glycans, as they were only labeled by ST6Gal1 and ST3Gal4 but not ST3Gal1 and ST6GalNAc1

FIG. 4 shows maximal labeling of asialofetuin (AF) was achieved with 1.2 μM of CMP-Cy5-SA. Labeling of 5 μg asialofetuin (Sigma Aldrich, St. Louis, MO) by 0.2 μg of ST3Gal1 or ST6Gal1 with variable amounts of CMP-Cy5-SA in 30 μL of reaction volume. The labeling reactions were incubated at 37° C. for 30 minutes and were terminated by 6×SDS gel loading buffer. Labeled samples were separated on SDS-PAGE and visualized by trichloroethanol (TCE) imaging (top panel) and fluorescent imaging (lower panel). Visible labeling on asialofetuin by ST3Gal1 was observed at 4.11 picomoles (pmol) of CMP-Cy5-SA and maximal labeling by ST3Gal1 was achieved at 37.0 pmol of CMP-Cy5-SA (1.2 Visible labeling on asialofetuin by ST6Gal1 was observed at 1.37 pmol of CMP-Cy5-SA level and maximal labeling by ST6Gal1 was achieved at 37.0 pmol of CMP-Cy5-SA (1.2 Asialofetuin was partially degraded and the degradation products are visible in the fluorescent image. M, Western molecular marker (Bio-Rad Laboratories, Hercules, CA).

FIG. 5 shows the lower limit of detection for asialofetuin labeled by ST3Gal1 and ST6Gal1 is at a sub-microgram level. Variable amounts of asialofetuin (Sigma-Aldrich, St. Louis, MO), as indicated, were labeled by 0.2 μg of ST3Gal1 or ST6Gal1 with 0.2 nanomoles (nmol) of CMP-Cy5-SA in 30 μL of reaction volume. The labeling reactions were incubated at 37° C. for 30 minutes and terminated by 6×SDS gel loading buffer. The samples were separated on SDS-PAGE and visualized by trichloroethanol (TCE) imaging (top panel) and fluorescent imaging (lower panel). In both cases, 0.37 μg of asialofetuin is the limit that may be detected. However, due to the significant degradation of asialofetuin, the actual limit for detection could be much lower. ST6Gal1 (61) showed significant self-labeling. M, Western molecular marker (Bio-Rad Laboratories, Hercules, CA).

FIG. 6 shows the lower limit of detection for recombinant MUC1 labeled by ST3Gal1 is at a nanogram level. Variable amounts of MUC1 (R&D Systems, Minneapolis, MN), as indicated, were labeled by 0.2 μg of ST3Gal1 or ST6Gal1 with 0.2 nmol of CMP-Cy5-SA in 30 μL of reaction volume. The labeling reactions were incubated at 37° C. for 30 minutes and terminated by 6×SDS gel loading buffer. The samples were separated on SDS-PAGE and visualized by trichloroethanol (TCE) imaging (top panel), fluorescent imaging (middle panel), and silver staining (lower panel). ST3Gal1 strongly labeled MUC1 with the lower limit of detection around 0.012 μg. ST6Gal1 did not label MUC1 but labeled itself. ST3Gal1 did not label itself but was labeled by ST6Gal 1. M, Western molecular marker (Bio-Rad Laboratories, Hercules, CA).

FIG. 7 shows the optimal amount of ST3Gal1 and ST6Gal1 for asialofetuin (AF) labeling is below 1 μg. 5 μg of asialofetuin (Sigma Aldrich, St. Louis, MO) was labeled by variable amounts of ST3Gal1 or ST6Gal1 with 0.2 nmol of CMP-Cy5-SA in 30 μL of reaction volume. The labeling reactions were incubated at 37° C. for 30 minutes and terminated by 6×SDS gel loading buffer. Labeled samples were separated on SDS-PAGE and visualized by trichloroethanol (TCE) imaging (top panel) and fluorescent imaging (lower panel). ST3Gal1 at 0.063 μg level showed clear visible labeling on asialofetuin and at 0.5 μg level achieved maximal labeling. ST6Gal1 at 0.016 μg level showed clear visible labeling on asialofetuin and at 0.25 μg level achieved maximal labeling. Asialofetuin was partially degraded and the degradation products are visible by the labeling. M, Western molecular marker (Bio-Rad Laboratories, Hercules, CA).

FIG. 8A-FIG. 8E shows substrate specificities of various fucosyltransferases and exemplary strategies for their substrate glycans labeling. FIG. 8A. FUT2 substrate specificity and its substrate glycan labeling. FIG. 8B. An exemplary strategy for labeling lactosamine (LN) by α-3 linkage specific FUT6 and FUT9. FIG. 8C. An exemplary strategy for labeling sialyl-lactosamine (sLN) by α-3 linkage specific FUT6 and FUT7. FIG. 8D. An exemplary strategy for detecting high-mannose glycans. Man9 type high-mannose glycan is depicted here, but other types of high-mannose glycans may also be detected as long as a terminal β-2 linked GlcNAc is installed on the α-3 arm by Mannosyl (α-1,3-)-Glycoprotein β-1,2-N-Acetylglucosaminyltransferase (MGAT1) (prior to probing the sample by adding fluorescent fucose in the presence of FUT8). FIG. 8E. An exemplary strategy for detecting core-fucosylation. The sialic acids and galactose residues on complex N-glycans of a glycoprotein sample are first removed enzymatically using neuraminidase and galactosidase before fucosylation may be measured by probing the sample by adding fluorophore-conjugated fucose in the presence of FUT8Flexible sugar residues are depicted with dashed outlines.

FIG. 9A-FIG. 9B show exemplary results of probing substrate glycans of various fucosyltransferases on fetuin and asialofetuin. FIG. 9A. Probing substrate glycans of fucosyltransferases with guanosine 5′-diphosphate activated Cy5 conjugated fucose (GDP-Cy5-Fuc). Samples of fetuin (Fet) or asialofetuin (A-fet) of fetal bovine origin were probed with fucosyltransferase (FUT) 2 (F2), FUT6 (F6), FUT7 (F7), and FUT9 (F9) in the presence of GDP-Cy5-Fuc. FUT9 labeled itself (indicated by an arrow in the gel). FIG. 9B. Tolerance of Alexa-Fluor®555 (555), Alexa-Fluor®488 (488), and Cy5 by the indicated fucosyltransferases. All reactions were incubated at 37° C. for 30 minutes and then separated on SDS-PAGE and imaged with silver staining (upper panels) and fluorescent imager (lower panels). M, molecular marker.

FIG. 10A-FIG. 10D show exemplary results of probing core-6 fucosylation on antibodies by FUT8 incorporation of fluorophores. FIG. 10A. Results of probing substrate glycans of FUT8 (F8) and FUT9 (F9) on Cantuzumab, an anti-Muc1 therapeutic antibody, and NIST reference mAb 8671 using guanosine 5′-diphosphate activated Alexa-Fluor®555 conjugated fucose (GDP-AF555-Fuc) (555), GDP-Cy5-fucose (Cy5), and guanosine 5′-diphosphate activated Alexa-Fluor®488 conjugated fucose (GDP-AF488-Fuc) (488) as donor substrates. Cantuzumab was expressed in FUT8 knockout cells and is devoid of core-6 fucose. Molecular marker (MM) is a prestained Western molecular marker (Bio-Rad Laboratories, Hercules, CA). FUT9 (50 kDa) showed self-labeling (indicated by an arrow in the gel). The product of each reaction was separated using SDS-PAGE and imaged with trichloroethanol (TCE) staining (upper panel) and fluorescent imager (lower panel). Labeling on the light chains of the antibodies by Alexa-Fluor®555 is likely the result of non-specific staining due to the use of unpurified GDP-AF555-Fuc that contained free Alexa-Fluor®555. FIG. 10B. Gly-Q™ analysis of the Cantuzumab samples prepared side-by-side for the samples in FIG. 10A indicates that FUT8 modified G0 and G1[6], and, FUT9 modified G1[6] and G1[3]. FIG. 10C. Gly-Q™ analysis (Prozyme, Inc., Agilent Technologies, Santa Clara, CA) of the NIST mAb samples prepared side-by-side for the samples in FIG. 10A indicates that FUT8 had no modification on any glycan species and FUT9 modified G1[6]F^{c} and G2F^{c}. FIG. 10D. Schematics of the glycan structures analyzed in FIG. 10B and FIG. 10C.

FIG. 11A-FIG. 11D show detecting high mannose glycans on glycoproteins by FUT8.

FIG. 11A. Detecting Man5 on RNase B and Man3 on recombinant H1N1 neuraminidase monomer (Neu). Samples were pretreated with MGAT1, FUT8, β-1,4-Galactosyltransferase 1 (B4GalT1) and ST6Gal1 with their respective unmodified donor substrates (indicated with +). The pretreated samples were then labeled by FUT8 with GDP-Cy5-Fuc or ST6Gal1 (61) with CMP-Cy5-SA. All samples were separated on SDS-PAGE and visualized by silver staining and fluorescent imaging. The middle and lower panels belong to a same image with different contrasts. Only MGAT1 modified sample was strongly labeled by FUT8. FIG. 11B. Representative Gly-Q™ analysis data of pretreated samples of RNase B (RB), showing that Man5 (M5) was sequentially modified by MGAT1 and FUT8. FIG. 11C. Representative Gly-Q™ analysis data of pretreated samples of Neu, showing that both Man3 (M3) and M3F^{c} were modified by MGAT1, but only M3N was further modified by FUT8. FIG. 11D. Molecular structures of Man3 and Man5 and their derivatives for FUT8 and ST6Gal1 labeling. While the (32 linked GlcNAc introduced by MGAT1 on α3 arm is essential for FUT8 recognition, the mannose residues in lighter shade on α6 arm are flexible for substrate recognition. Further elongation of the α3 arm with B4GalT1 renders the glycan to be the substrate glycan for ST6Gal1.

FIG. 12A-FIG. 12B show sequential conversion of Man5 by MGAT1, B4GalT1, ST6Gal1, and FUT8. FIG. 12A. Gly-Q™ analysis of sequential samples in FIG. 11, panel A. The Man5 glycan (M5) on RNase B (RB) was the only glycan that was converted by these enzymes. It was sequentially converted by MGAT1 and B4GalT1 to M5N and M5NG, respectively. FUT8 was able to convert M5N to M5NF. ST6Gal1 was able to convert M5NG to M5NGS. FIG. 12B. Schematics of the structures of the glycans that were converted by the sequential enzymatic reactions in FIG. 12A.

FIG. 13 shows the effect of sequential modification of Man3 on recombinant H1N1 influenza viral neuraminidase by MGAT1, B4GalT1, ST6Gal1 on FUT8 labeling. Recombinant 1918 H1N1 viral neuraminidase monomer (Mo) and dimer (Di) were used as substrates for the glycan modification. Pretreatment of the substrates for FUT8 labeling was performed by incubating the substrates with MGAT1, B4GalT1, and ST6Gal1 (in the presence of their donor substrates) for 30 minutes at 37° C. Labeling was achieved by incubating the pretreated samples with the labeling enzymes FUT8 and the fluorescent donor substrate GDP-AF555-Fuc (555) or GDP-Cy5-Fuc (Cy5). For comparison, the dimeric neuraminidase was also pretreated with MGAT1 and FUT8 (in the presence of their donor substrates) at 37° C. for 30 minutes, and then labeled by incubating with B4GalT1 (B4) and ST6Gal1 (61) in the presence of CMP-AF555-SA or CMP-Cy5-SA.

FIG. 14A-FIG. 14C show the effects of simultaneous labeling by FUT9 (F9) or FUT7 (F7), and ST6Gal1 (61) or ST3Gal2 (32) on HEK293 cell extracts. HEK293 cell extracts were labeled with Alexa-Fluor® 555-conjugated fucose (AF555-Fuc) by an indicated fucosyltransferase (lane a) or Cy5-conjugated sialic acid (Cy5-SA) by an indicated sialyltransferase (lane b) or by both (lane c). Recombinant C. perfringens neuraminidase (Neu) was added into some lanes as indicated to remove existing terminal sialic acids. All reactions were separated on SDS-PAGE. FIG. 14A. TCE image of the gel. FIG. 14B. Fluorescent image of the gel from both green and red channels. FUT9 labeled by itself, ST3Gal2 labeled by FUT9, and FUT7 labeled by ST6Gal1 are indicated by arrows. FIG. 14C. Single channel images of FUT9 plus ST6Gal1, and FUT9 plus ST3Gal2 double labeling. M, prestained Protein Ladder (Bio-Rad Laboratories, Hercules, CA), visible from the red channel in FIG. 14B.

FIG. 15A shows the nine-carbon backbone of sialic acid in the α configuration. FIG. 15B shows a diagram of Cytidine Monophosphate Activated Fluorophore-Conjugated Sialic Acid (CMP-f-SA).

FIG. 16A shows the six-carbon backbone of fucose in the α configuration. FIG. 16B shows a diagram of Guanidine Diphosphate Activated Fluorophore-Conjugated Fucose (GDP-f-Fuc).

FIG. 17 shows exemplary results of a kinetic study of the labeling by FUT9. Mouse IgG (R&D Systems, Minneapolis, MN) was digested with Endo S thoroughly and labeled with 0.3 μg of FUT9 and 0.2 nmol of GDP-Cy5-Fuc in 30 μl at 37° C. for the indicated time points. The glycans were also pretreated with B4GalT2 for 5 minutes at 37° C. and then labeled in the same way. The reactions were separated on 15% SDS PAGE. B4GalT2 pretreatment converted all G1′ to G2′. Maximal labeling on G1′ and G2′ were achieved around 30 minutes. FUT9 showed self-labeling. B4GalT2 was labeled as well and maximal labeling was achieved at 20 minutes. The heavy chain (HC) and light chain (LC) were visible in the TCE image. M, Western blot marker (Bio-Rad Laboratories, Hercules, CA).

FIG. 18A-FIG. 18B show characterization of the labeled glycans on fetuin (Fet) and asialofetuin (AF) with PNGase F and FUCA1. FIG. 18A. PNGase F treatment of the labeled samples. AF was labeled by FUT2 (F2), FUT6 (F6) and FUT9(F9) with Alexa Fluor® 555 (green) or Cy5 (red). Fet was labeled by FUT7 (F7) with Alexa Fluor® 488 (blue). Labeled samples were then treated with PNGase F to release the glycans. FIG. 18B. Effect of FUCA1 on the labeling. Samples without or with FUCA1 treatment were labeled by the indicated enzymes with Cy5. All samples were separated on 15% SDS-PAGE and imaged by silver staining, TCE staining and fluorescent imaging as indicated. M, Western blot molecular marker.

FIG. 19A shows establishment of reference glycan standards. G0 was first labeled by FUT8 and then enzymatically converted to 5 other glycans, including G2F2 that carries Lewis X structure and A2[3]F2 that carries sialyl Lewis X structure. The labeled glycans were then separated on a 17% SDS-PAE (about 0.25 ng each of the labeled glycan was loaded in each lane). FIG. 19B shows characterization of glycans on Cantuzumab (CAN) and NIST mAb (NIST). Glycans on Cantuzumab and NIST mAb (2.5 μg per sample) were released by either Endo S or PNGase F and then labeled by FUT9 or FUT8 directly or after treatment of B4GalT1. The labeled glycans were separated on a 17% SDS-PAGE together with some of the glycan standards generated in FIG. 19A (panel a). Endo S released glycans lack the core GlcNAc residue at the reducing ends (with dashed lines and light shades) and are indicated with prime symbols. For better viewing the glycan separation, labeling on PNGase F released glycans by FUT9 was repeated in panel b. Nomenclature is consistent with FIG. 10D. The galactose residue in the monogalactosylated glycans in FIG. 19B can be in either arm, but only one is presented.

FIG. 20 shows sequential conversion of molecular structures Man5 by MGAT1, B4GalT1, ST6Gal1, and FUT8, that is the glycans that were converted by the sequential enzymatic reactions of Example 3. In the nomenclature, M is mannose, N is GlcNAc, G is Gal, S is sialic acid, and F′ is core fucose.

FIG. 21A-FIG. 21D shows exemplary kinetic comparisons of fucosylation and sialylation on Lewis X (Le^{x}) and sialyl Lewis X (sLe^{x}) synthesis. FIG. 21A. Schematic view of the enzymatic steps for Le^{X} and sLe^{X} synthesis on the antibody glycan G0. The glycan short names follow that of FIG. 10. FIG. 21B Kinetic comparison of galactosylation on G0 by B4GalT1, sialyation on G2 by ST3Gal4 and ST6Gal1, and fucosylation on A2[3] by FUT7. The reaction time for B4GalT1 was 10 minutes. The reaction time for ST3Gal4, FUT7 and ST6Gal1 was 5 hours. In each case, the labeling enzyme was subject to a 3-fold serial dilution starting from 1 μg each of B4GalT1, FUT7 and ST6Gal1, and 3 μg ST3Gal4. FIG. 21C. Kinetic comparison of sLeX synthesis by FUT6 and FUT7 from A2[3]. Reaction time for both cases was 20 minutes. Both FUT6 and FUT7 were subject to 6-fold serial dilution starting from 1 μg. FIG. 21D. Kinetic comparison of LeX synthesis by FUT6 and FUT9 from G2. Reaction time for both cases was 20 minutes. Both FUT6 and FUT9 were subject to 6-fold serial dilution starting from 1 μg.

FIG. 22A-FIG. 22C show quantification of Le^{a} and its intermediate products. FIG. 22A shows a scheme of the synthesis of Le^{a} using B3GalT2, ST3Gal3 and FUT3. FIG. 22B shows the step-by-step synthesis of Le^{a} and a 2-fold serial dilution of G0f. Lane 1 is labeled G0 (G0f) and lane 2 is labeled G2 (G2f). Lane 3 to 5 contain the intermediate reactions of converting G0f to the final Le^{a} carrying glycan A1 G1[3]F1f with the indicated enzymes. Lane 6 to lane 13 is a 2-fold serial dilution of G0f starting from 25 ng (19 pmol). The exposure time is 240 ms. The mass inputs of the serial dilution and all the band intensities were listed below the picture. FIG. 22C. The response curve of the serial dilution in FIG. 22B. The slope of the line represents the signal to mass ratio of G0f, which allows the quantification of the bands from lanes 1 to 5.

FIG. 23 shows nomenclature for non-reducing end monosaccharides used in Example 4 and in the detailed description of this application. A non-reducing end monosaccharide of an N-glycan is represented with a single capital letter followed by the number of the monosaccharide present in the glycan. If necessary, a number in a parenthesis is used to specify the linkage of the monosaccharide. A prime symbol (′) is used to indicate if a monosaccharide is conjugated to a fluorophore. A short name of an N-glycan is a composite of the all the non-reducing end monosaccharides.

FIG. 24A-FIG. 24E shows relative mobilities of various Cy5-labeled glycans and their enzymatic synthesis. All glycans were enzymatically synthesized based on N2 (also referred to herein as G0). The Glycan ladders were composed by combining some of the labeled glycans. Except enzymatic reactions of FUT8, MGAT3 and MGAT5, all other enzymatic reactions resulted in one or two intermediates as that there are two or more branches in each glycan that allow enzymatic modification (see FIG. 25 for intermediates). The labeled glycans were separated on 17% gel and visualized with a fluorescent imager. FIG. 24A. Relative mobilities of glycans that are labeled at the core-fucose. The enzymes used for generating these glycans are listed on the top of the gel. FIG. 24B. Relative mobility change on N2f contributed by a bisecting GlcNAc introduced by MGAT3 (MT3) versus a β1-6G1cNAc introduced by MGAT5 (MT5). MGAT3 and MGAT5 were introduced in different orders. The second enzyme was introduced 30 minutes after the first enzyme. Doubly modified product N3nf′ can only be observed when MGAT5 was introduced first. FIG. 24C. Relative mobility change on ST[6]G1 contributed by a bisecting GlcNAc versus a core-fucose. The enzymes used for generating these glycans are specified on the top of the gel in the specified order and they are MGAT3 (MT3), FUT8(FT8), B4GalT1 (B41) and ST6Gal1 (ST61).

FIG. 24D shows schemes for enzymatic generation of the labeled glycans in FIG. 24A and FIG. 24B. FIG. 24E shows schemes of enzymatic generation of the labeled glycans in FIG. 24C. Two fluorophore sialic acids can be introduced to each of the glycan, but only glycans with one Cy5-conjugated sialic acid were displayed in FIG. 24B, FIG. 24C, and FIG. 24E.

FIG. 25A-FIG. 25D show intermediates of enzymatically generated labeled glycans. All glycans were enzymatically extended from N2f′ that was labeled at the core-fucose by Fut8 with GDP-Cy5-Fuc. Enzymatic reactions were incubated at room temperature for 30 minutes (FIG. 25A), 90 minutes (FIG. 25B), or overnight (FIG. 25C) and were separated on a 17% SDS gel. Synthesis of N2nf′ and G3f were only introduced in FIG. 25B and FIG. 25C, respectively. Enzymes used for the conversion are indicated above the images. B41, B4GalT1; 61, ST6Gal1; 36, ST3Gal6; 34, ST3Gal4; 61, ST6Gal1; MT3, MGAT3; MT5, MGAT5, F9, Fut9. Short names of glycans and schemes for enzymatic generation of these glycans are indicated in FIG. 25D, according to the nomenclature of FIG. 23.

FIG. 26A-FIG. 26C show results of selecting ST6Gal1 and ST3Gal6 for glycan finger printing and their substrate concentration optimization. In all cases, labeling reactions were proceeded for 2 hours at 37° C. and then separated on indicated SDS-gel, and, imaged by TCE staining (top panels) and fluorescent imaging (low panels). 33, ST3Gal3; 34, ST3Gal4; 36, ST3Gal6; 61, ST6Gal 1. ST3Gal6 showed self-labeling as indicated. FIG. 26A. Glycans from SARS2 RBD protein expressed in CHO cells were released by PNGase F and labeled by various sialyltransferases with CMP-Cy5-Sialic acid. Samples were also labeled with (+) or without (−) neuraminidase (Neu) pretreatment. The glycan ladder contained G2f′, N2f′ and S2[6]f′. FIG. 26B. Variable amounts of RBD protein were labeled by ST6Gal1 or ST3Gal6. FIG. 26C. Variable amounts of CMP-Cy5-Sialic acid were used to label RBD proteins by ST6Gal1 or ST3Gal6.

FIG. 27A-FIG. 27B show exemplary results of fingerprinting glycans released from various SARS2 Spike proteins with ST6Gal1 (red) and FUT8 (green). Glycans of various recombinant spike proteins released by PNGase F were labeled by ST6Gal1 and FUT8 with standard glycans (FIG. 27A), or, with glycan ladders (FIG. 27B) with (+) or without (−) pretreatment of neuraminidase. Labeled samples were separated on 17% SDS gel and imaged with regular protein imaging (upper panels) or fluorescent imaging (middle and lower panel in different contrasts). Bands in red are complex or hybrid glycans labeled by ST6Gal1 (via the incorporation of Cy5-conjugated sialic acid). Bands in green are oligo-mannose glycans labeled by FUT8 (via the incorporation of AlexaFluor 555-conjugated Fucose). For oligomannose labeling, MGAT1 and UDP-GlcNAc were also included in the labeling mix to convert the oligomannose glycans to the substrates for FUT8. Labeled glycans from Ribonuclease B, S′1[6]N1, S′1[6]G1 and S′1[6]G1f were run as references in FIG. 27A, and a glycan ladder with 10 labeled standard glycans was run in FIG. 27B. RS, RBD domain of Spike protein expressed in Sf21 cells; RC, RBD domain of Spike protein expressed in CHO cells; RH, RBD domain of Spike protein expressed in HEK293 cells; SC, whole Spike protein expressed in CHO cells; S1H, 51 protein expressed in HEK293 cells; SH, Spike protein expressed in HEK293 cells.

FIG. 28A-FIG. 28C shows mobility shift of band d and band 5 of FIG. 27 by enzymatic modification. Glycans of the recombinant RBD expressed in Sf21 cells (RS) and whole spike protein expressed in HEK293 cells (SH) along with that of RNase B released by PNGase F were first labeled by FUT8 with GDP-Cy3-fucose together with MGAT1 and then further treated with B4GalT1 and ST6Gal1 (indicated with + and − signs). RNase B known to contain Man5 (M3) was used as a control. Samples were run on 17% SDS gel visualized by TCE imaging (upper panel) and fluorescent imaging (lower panel) (FIG. 28A). The scheme for labeling and molecular conversion of MAN3 (M2) is shown in FIG. 28B. The scheme for labeling and molecular conversion of M3 is shown in FIG. 28C. The labeled bands from SH and RNase B are at same the position, suggesting that the glycan released from SH is indeed M3. The spacing between the labeled band of RS and SH suggests that the glycan released from RS is M2. The shifts on the labeled bands from RS and SH caused by B4Galt 1 and ST6Gal1 are the same, further confirming that the glycan released from RS is M2.

FIG. 29 shows fingerprinting by ST3Gal6 and ST6Gal1 on various SARS2 Spike proteins. Glycans of various recombinant spike proteins released by PNGase F with (+) or without (−) pretreatment of neuraminidase (Neu) were labeled by ST3Gal6 and ST6Gal1 together with FUT8 and separated on 17% SDS gel. The gel was then imaged with silver staining (upper panel) or fluorescent imaging (middle and lower panel in different contrasts). Bands in red are complex or hybrid glycans labeled by ST6Gal1 or ST3Gal6 (via the incorporation of Cy5-conjugated sialic acid). Bands in green are oligo-mannose glycans labeled by FUT8 (via the incorporation of AlexaFluor 555-conjugated Fucose). For oligomannose labeling, MGAT1 and UDP-GlcNAc were also included in the labeling mix to convert the glycans to the substrates for FUT8. RC, RBD domain of Spike protein expressed in CHO cells; RH, RBD domain of Spike protein expressed in HEK293 cells; SH, whole Spike protein expressed in HEK293 cells.

### DETAILED DESCRIPTION OF ILLUSTRATIVE EMBODIMENTS

This disclosure describes fluorophore-conjugated sialic acids and fluorophore-conjugated fucose and methods that include enzymatic incorporation of a fluorophore-conjugated sialic acid or a fluorophore-conjugated fucose or both to label and detect N- and O-glycans on glycoproteins. These compositions and methods allow for the detection of specific glycans without the laborious gel blotting and chemiluminescence reactions used in Western blotting and the detection of a glycan in its native state.

### Sialylation

Sialylation is catalyzed by multiple sialyltransferases (Harduin-Lepers et al. Glycobiology 15, 805-817 (2005)). N-glycan sialylation typically occurs on galactose (Gal) residues and is mediated by the N-glycan specific α-2,6-sialyltransferase 1 (ST6Gal1) (Weinstein et al. J Biol Chem 262, 17735-17743 (1987)) and α-2,3-sialyltransferase 4 (ST3Gal4) (Mereiter et al. Biochim Biophys Acta 1860, 1795-1808 (2016)). O-glycans may also be sialylated on Gal residues by 0-glycan specific α-2,3-sialyltransferase 1 and 2 (ST3Gal1 and ST3Gal2) and on O-GalNAc residues by a family of α-N-acetylgalactosaminide α-2,6-sialyltransferases (ST6GalNAc) (Ju et al. Cancer Res 68, 1636-1646 (2008), Kitagawa et al. J Biol Chem 269, 1394-1401 (1994)). Among all ST6GalNAcs, ST6GalNAc4 is strictly active on sialylated T antigen and is responsible for disialylated T antigen expression (Harduin-Lepers et al. Glycobiology 15, 805-817 (2005)).

Despite the abundance of N- and O-glycans and their important biological functions, research on these glycans has been hampered by the lack of high affinity (Ambrosi et al. Org Biomol Chem 3, 1593-1608 (2005)) and specific binding reagents (Geisler et al. Glycobiology 21, 988-993 (2011), Sterner et al. ACS Chem Biol 11, 1773-1783 (2016)). The emergence of click chemistry (Kolb et al. Angew Chem Int Ed Engl 40, 2004-2021 (2001)) provided a new avenue for glycan labeling (Codelli et al. J Am Chem Soc 130, 11486-11493 (2008), Hsu et al. Proc Natl Acad Sci USA 104, 2614-2619 (2007)). Subsequently, using enzymatic incorporation of clickable monosaccharides, specific glycan labeling became feasible (Chaubard et al. JAm Chem Soc 134, 4489-4492 (2012), Mbua et al. Angew Chem Int Ed Engl 52, 13012-13015 (2013), Wu et al. Glycobiology 28, 69-79 (2018)). More recently, glycan labeling via direct incorporation of biotinylated sialic acids using ST6Gal1 (Capicciotti et al. J Am Chem Soc 139, 13342-13348 (2017)) and ST6GalNAc4 (Wen et al. ACS Cent Sci 4, 451-457 (2018)) has been reported.

### Fluorophore-Conjugated Sialic Acid

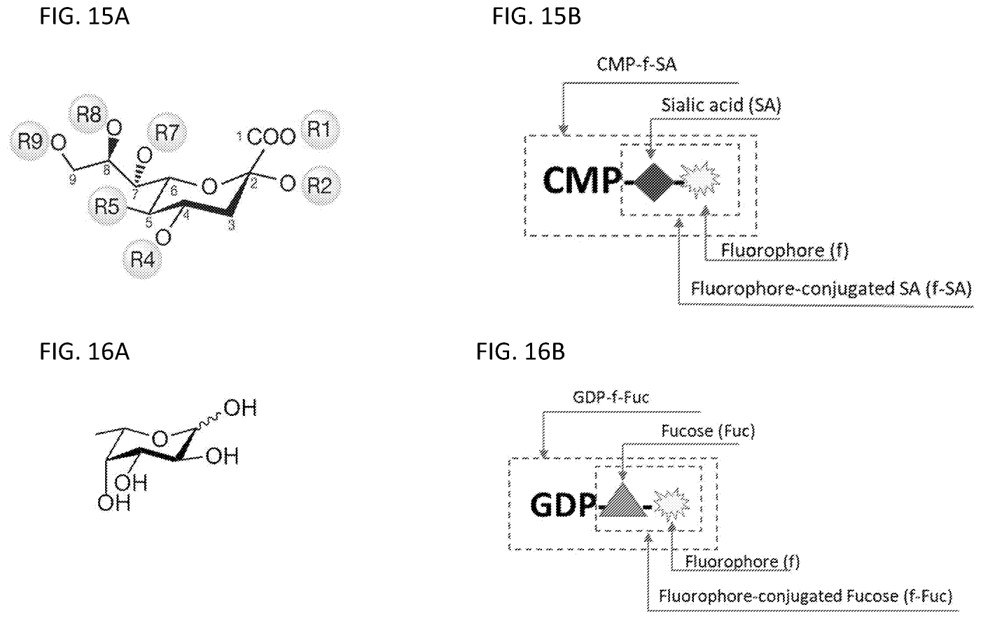

In one aspect, this disclosure describes a fluorophore-conjugated sialic acid and a composition including the fluorophore-conjugated sialic acid. The nine-carbon backbone common to all known sialic acids is shown in the α configuration in FIG. 15A and is reproduced, below.

In some embodiments, the fluorophore-conjugated sialic acid preferably includes an activated fluorophore-conjugated sialic acid. As used herein, an “activated” sialic acid means a nucleotide-conjugated sialic acid. For a sialic acid to enter into an oligosaccharide biosynthesis process, the sialic acid must be activated by conjugation to a monophosphate nucleoside (typically from a cytidine triphosphate).

In some embodiments, the activated fluorophore-conjugated sialic acid preferably includes a cytidine monophosphate activated fluorophore-conjugated sialic acid (CMP-f-SA). A diagram of CMP-f-SA is shown in FIG. 15B and is reproduced below

In some embodiments, the sialic acid includes N-acetyl-neuraminic acid (Neu5Ac or NANA), 2-keto-3-deoxynononic acid (Kdn), N-glycolylneuraminic acid (Neu5Gc), neuraminic acid (Neu), or 2-deoxy-2,3-didehydro-Neu5Ac (Neu2en5Ac), or combinations thereof.

In some embodiments, the sialic acid preferably includes the α-anomer, that is, the form of sialic acid that is bound to glycans.

The fluorophore may include any suitable fluorophore that allows the sialic acid to be incorporated into a glycan by a sialyltransferases, as further described below. In some embodiments, the fluorophore includes Alexa Fluor® 488, Alexa Fluor® 555, or Cy5. In some embodiments, the fluorophore for the fluorophore-conjugated sialic acid may be selected based on the sialyltransferase to be used. (See, for example, Table 1.)

### Methods of Using a Fluorophore-Conjugated Sialic Acid

This disclosure further describes methods for using a fluorophore-conjugated sialic acid, preferably, an activated fluorophore-conjugated sialic acid including, for example, CMP-f-SA.

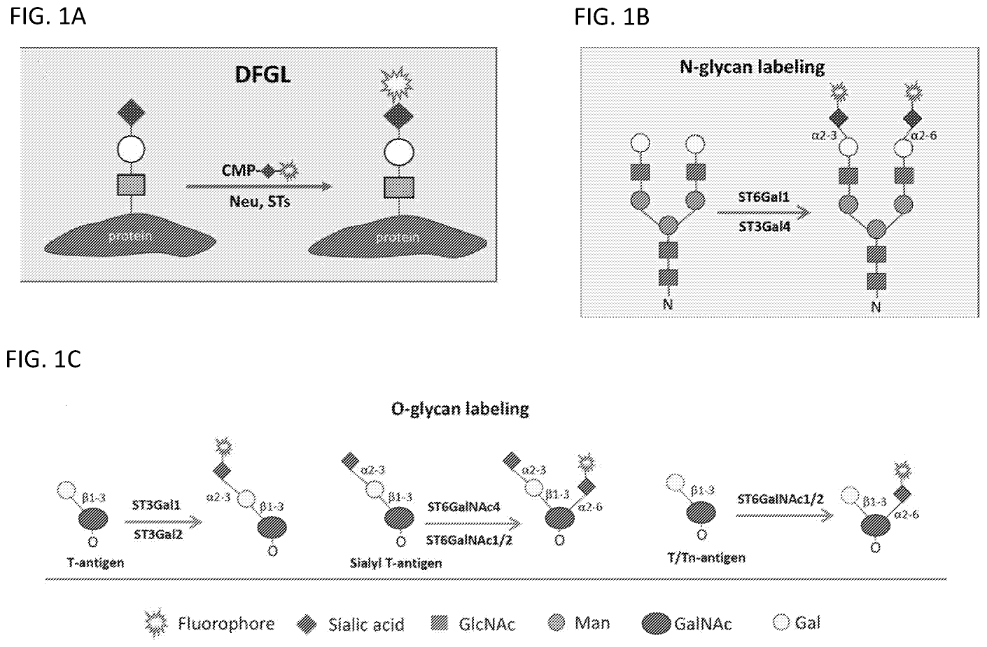

In some embodiments, the fluorophore-conjugated sialic acid is used in a method for specific glycan labeling (also referred to herein as Direct Fluorescent Glycan Labeling (DFGL)), in which a fluorophore-conjugated sugar is directly attached to a target glycan via a specific enzyme (for example, a specific sialyltransferase). Some exemplary methods are shown in FIG. 1 and described in Example 1.

The method may include using any sialyltransferase that is able to incorporate the fluorophore-conjugated sialic acid into a glycan. In some embodiments, the sialyltransferase may include, for example, ST3Gal 1, ST3Gal2, ST3Gal3, ST3Gal4, ST3Gal5, ST3Gal6, ST6Gal 1, ST6Gal2, ST6GalNAc1, ST6GalNAc2, ST6GalNAc3, ST6GalNAc4, ST6GalNAc5, ST6GalNAc6, ST8SIA1, ST8SIA2, ST8SIA3, ST8SIA4, ST8SIA5, or ST8SIA6, or a combination thereof.

In some embodiments, the method includes mixing a target glycan with an activated fluorophore-conjugated sialic acid (for example, CMP-f-SA), and the sialyltransferase. In some embodiments the target glycan, the activated fluorophore-conjugated sialic acid, and the sialyltransferase may be mixed in a buffer.

In some embodiments, the method includes mixing a target protein (for example, a glycoprotein) which includes a glycan with an activated fluorophore-conjugated sialic acid (for example, CMP-f-SA), and the sialyltransferase. In some embodiments the target protein, the activated fluorophore-conjugated sialic acid, and the sialyltransferase may be mixed in a buffer.

In an exemplary embodiment, the buffer includes 25 mM Tris pH 7.5 and 10 mM MnC12.

In some embodiments, the target glycan or the target protein, the activated fluorophore-conjugated sialic acid, and the sialyltransferase are incubated together for at least 1 minute, at least 5 minutes, at least 10 minutes, at least 15 minutes, at least 20 minutes, at least 25 minutes, or at least 30 minutes. In some embodiments, the target glycan or the target protein, the activated fluorophore-conjugated sialic acid, and the sialyltransferase are incubated together for up to 10 minutes, up to 15 minutes, up to 20 minutes, up to 25 minutes, up to 30 minutes, up to 1 hour, up to 2 hours, up to 24 hours, or up to 48 hours. In an exemplary embodiment, the target glycan or the target protein, the activated fluorophore-conjugated sialic acid, and the sialyltransferase are incubated together for 30 minutes.

The target glycan or the target protein, the activated fluorophore-conjugated sialic acid, and the sialyltransferase may be incubated together at any temperature at which the sialyltransferase is able to incorporate the fluorophore-conjugated sialic acid into the target glycan or into the target glycan or a glycan of the target protein. In some embodiments, the target glycan or the target protein, the activated fluorophore-conjugated sialic acid, and the sialyltransferase may be incubated together at a temperature of at least 20° C., at least 25° C., at least 28° C., or at least 30° C. In some embodiments, the target glycan or the target protein, the activated fluorophore-conjugated sialic acid, and the sialyltransferase may be incubated together at a temperature of up to 32° C., up to 35° C., up to 37° C., up to 40° C., up to 45° C., or up to 50° C. In an exemplary embodiment, the target glycan or the target protein, the activated fluorophore-conjugated sialic acid, and the sialyltransferase may be incubated together at 37° C.

In some embodiments, including when the target glycan or a glycan of the target protein includes a preexisting sialic acid, the method includes adding a C. perfringens neuraminidase to the mixture including the target protein, the activated fluorophore-conjugated sialic acid, and the sialyltransferase. In some embodiments, the C. perfringens neuraminidase may include recombinant C. perfringens neuraminidase. As further described in Example 1, recombinant C. perfringens neuraminidase showed no activity on fluorophore-conjugated sialic acids but was able to remove natural (that is, non-fluorophore-conjugated) sialic acids from the glycans of target proteins. In some embodiments, at least 0.01 microgram (μg), at least 0.05 μg, or at least 0.1 μg of C. perfringens neuraminidase may be added. In some embodiments, up to 0.05 μg, up to 0.1 μg, up to 0.5 μg, or up to 1 μg of C. perfringens neuraminidase may be added. In an exemplary embodiment, 0.1 μg of recombinant C. perfringens neuraminidase is added into the mixture including the target protein, the activated fluorophore-conjugated sialic acid, and the sialyltransferase. In some embodiments, the C. perfringens neuraminidase may be used to remove a non-fluorophore-conjugated sialic acid, allowing for a fluorescent sialic acid to be added to the target glycan or the target protein, at a site which would otherwise not have been available.

In some embodiments, the method includes separating components of the mixture including the target glycan or the target protein, the fluorophore-conjugated sialic acid, the sialyltransferase, and the optional C. perfringens neuraminidase. In some embodiments, the components of the mixture may be separated using protein gel electrophoresis including, for example, sodium dodecyl sulfate-polyacrylamide gel electrophoresis (SDS-PAGE), capillary gel electrophoresis, isoelectric focusing electrophoresis, etc.

In some embodiments, as exemplified in Example 4, and as further discussed below, prior to separating the components, the method may further include cleaving a glycan including the fluorophore-conjugated sialic acid from the target protein. In such embodiments, as further discussed below, the labeled glycans cleaved from the target protein may be separated to assess mobility of the labeled glycans.

In some embodiments, the method includes imaging the separated components. For example, in an exemplary embodiment, when the components are separated in an SDS-PAGE gel, they may be imaged by fluorescent imaging including, for example, using a fluorescent imager. Additionally or alternatively, the components may be imaged using trichloroethanol (TCE) staining, silver staining, or Coomassie blue staining, or a combination of these methods. That is, in contrast to previous methods which relied on Western blotting to visualize the components, a combination of imaging methods may be used.

When the glycans have not been cleaved from a target protein, separating the mixture and then imaging the separated components allows assessment of the labeling of the target protein. This process is also referred to herein as the Direct Fluorescent Glycan Labeling (DFGL) method.

When the glycans have been cleaved from a target protein, separating the mixture and imaging the labeled glycans allows for characterization of a glycan or of the glycosylation of a glycoprotein by assessing their mobility, as further described below.

The Direct Fluorescent Glycan Labeling (DFGL) method provides several advantages over detection of incorporation of a clickable sugar or a biotinylated sugar. First, the method is more convenient because it involves only a single enzymatic reaction step and allows direct imaging of separated samples without time-consuming membrane transfer and the chemiluminescence reaction required by the aforementioned alternative sugars.

Second, the method eliminates side effects caused by click chemistry reagents, such as oxidative cleavage of target proteins by copper ions and non-specific click reactions, by removal of these reagents before labeling reaction.

Third, because the fluorophores are specifically introduced by enzymatic reactions, the method virtually eliminates all non-specific background staining.

Fourth, DFGL allows direct imaging (for example, without binding to streptavidin-HRP) and/or combinations of different ways of imaging, methods that are not possible with clickable sugars or biotinylated sugars.

Additionally, in contrast to mass spectrometry analysis, in some embodiments, DFGL permits analysis and detection of a glycan in its native state—that is, without cleaving glycans from a glycoprotein or glycolipid. Detection of a glycan in its native state may provide valuable information about the whole glycan structure (as opposed to a singly glycan epitope). As further discussed below, in cases when further characterization of a glycan epitope is desired, a glycan may, alternatively, be released from the target glycoprotein or glycolipid, and its mobility analyzed.

### Fucosylation

Fucose is usually located at the non-reducing ends of various glycans on glycoproteins, and it constitutes important glycan epitopes. Detecting the substrate glycans of fucosyltransferases is important for understanding how these glycan epitopes are regulated in response to different growth conditions and external stimuli.

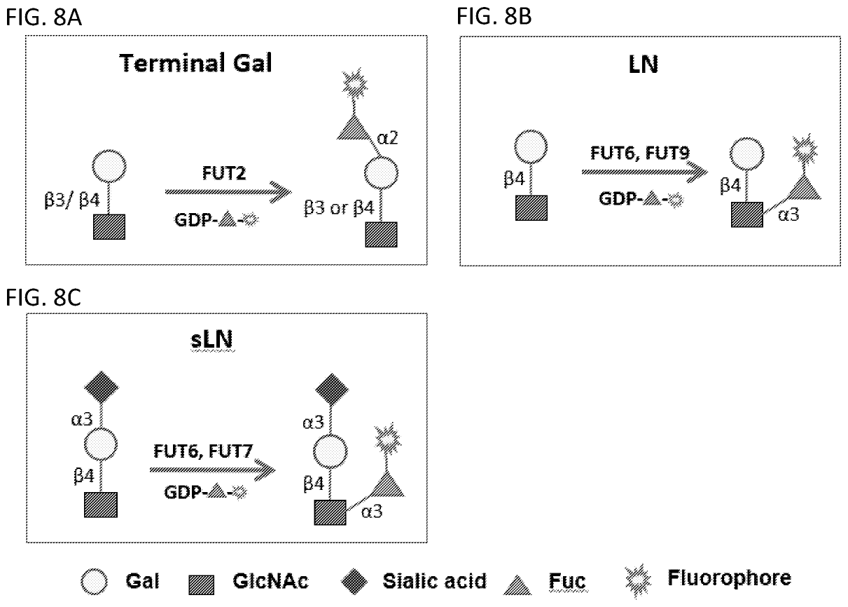

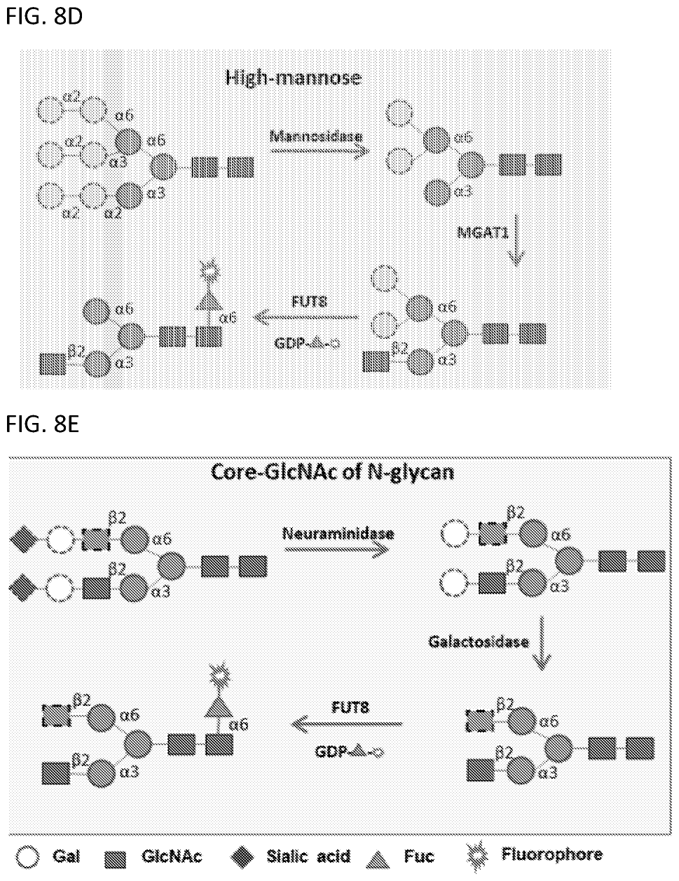

Well-known fucosylated glycans include blood group H-antigen, Lewis X structures, and core fucosylated N-glycan. As shown in FIG. 8, these fucosylated glycans are generated by various fucosyltransferases (Ma et al. Glycobiology 16, 158R-184R (2006)). H-antigen on red blood cells contains an α1-2 linked fucose introduced by FUT1 and FUT2 (Kelly et al. J Biol Chem 270, 4640-4649 (1995)). Lewis X structure is a trisaccharide (Galβ1-4[Fucα1-3] GlcNAc) that has a fucose residue linked to a GlcNAc residue through an α1-3 linkage. Lewis X structure may be sialylated at the Gal residue and becomes sialyl-Lewis X structure (Neu5Acα2-3Galβ1-4[Fucα1-3] GlcNAc) that is the ligand for E-selectin and is essential for lymphocyte extravasation (Nelson et al. J Clin Invest 91, 1157-1166 (1993)). The α-3 linked fucose on Lewis X and sialyl-Lewis X structures is introduced via several fucosyltransferases including FUT6, FUT7, and FUT9 (Mondal et al. J Biol Chem 293, 7300-7314 (2018)). Among these enzymes, FUT7 is strictly active on sialyllactosamine (Sasaki et al. J Biol Chem 269, 14730-14737 (1994)), FUT9 is strictly active on lactosamine (Brito et al. Biochimie 90, 1279-1290 (2008)), and FUT6 is active on both structures (Weston et al. J Biol Chem 267, 24575-24584 (1992)).

Fucosylation carried out by FUT9 is critical to ricin toxicity (Stadlmann et al. Nature 549, 538-542 (2017)). Core-6 fucosylation on the innermost GlcNAc of N-glycan introduced by FUT8 (Ihara et al. Glycobiology 16, 333-342 (2006)) plays critical role in the antibody-dependent cellular cytotoxicity (ADCC) of therapeutic antibodies (Jefferis Nat Rev Drug Discov 8, 226-234 (2009)). For FUT8 substrate recognition, an unmodified (31-2 linked GlcNAc residue introduced to the α-3 arm of N-glycan by MGAT1 (Kumar et al. Proc Natl Acad Sci USA 87, 9948-9952 (1990)) is used (Yang et al. J Biol Chem 292, 14796-14803 (2017)).

Lewis A structure (Galβ1-3[Fucα1-4] GlcNAc) and its sialylated version sialyl Lewis A are isomers of Lewis X and sialyl Lewis A structures, are fucosylated by FUT3 (Kukowska-Latallo et al. Genes Dev 4, 1288-1303 (1990)). Core-6 fucosylation on the innermost GlcNAc of N-glycan introduced by FUT8 (Ihara et al. Glycobiology 16, 333-342 (2006)) plays critical role in the antibody-dependent cellular cytotoxicity (ADCC) of therapeutic antibodies (Jefferis Nat Rev Drug Discov 8, 226-234 (2009)). For FUT8 substrate recognition, an unmodified β1,2-linked GlcNAc residue introduced to the α3 arm of N-glycan by MGAT1 (Kumar et al. Proc Natl Acad Sci USA 87, 9948-9952 (1990)) is critical (Yang et al. J Biol Chem 292, 14796-14803 (2017)).

Because of their important biological roles, cellular display of fucosylated glycan epitopes epitopes (Adey et al. Nature 500, 207-211 (2013)) are tightly regulated (Sackstein Immunol Rev 230, 51-74 (2009)). This regulation is believed to be achieved via the establishment of precursor glycan pools and controlled expression of key fucosyltransferases. Upon environmental stimuli, cells may quickly convert the precursor glycans to functional epitopes via the action of these enzymes. Therefore, it is important to detect the glycan epitopes as well as their precursor glycans.

### Fluorophore-Conjugated Fucose

In another aspect, this disclosure describes a fluorophore-conjugated fucose. The structure of fucose is shown in FIG. 16A and is reproduced below:

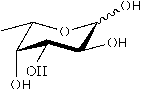

In some embodiments, the fluorophore-conjugated fucose preferably includes an activated fluorophore-conjugated fucose. As used herein, an “activated” fucose means a nucleotide-conjugated fucose. For a fucose to enter into an oligosaccharide biosynthesis process and be incorporated in a glycan by a fucosyltransferase, the fucose must be activated by conjugation to a guanosine diphosphate.

In some embodiments, the activated fluorophore-conjugated fucose preferably includes a guanosine diphosphate activated fluorophore-conjugated fucose (GDP-f-Fuc). A diagram of GDP-f-Fuc is shown in FIG. 16B and is reproduced below.

The fluorophore may include any suitable fluorophore that allows the fluorophore-conjugated fucose to be incorporated into a glycan by a fucosyltransferase, as further described below. In some embodiments, the fluorophore includes Alexa Fluor® 488, Alexa Fluor® 555, or Cy5. In some embodiments, the fluorophore is preferably conjugated to the c6 of fucose. In some embodiments, the fluorophore may be selected based on the fucosyltransferase to be used. (See, for example, Table 2.) As shown in Example 2, Cy5, AlexaFluor®555 and AlexaFluor® 488 conjugated fucoses were well tolerated by various fucosyltransferases

### Methods of Making a Fluorophore-Conjugated Fucose

The fluorophore-conjugated fucose may be prepared by any suitable method. In some embodiments, an activated fluorophore-conjugated fucose may be prepared via copper (I)-catalyzed azide-alkyne cycloaddition. For example, incubating a GDP-Azido-Fucose (GDP-N₃-Fuc) and an alkyne-conjugated fluorophore results in conjugation between the components via copper (I)-catalyzed azide-alkyne cycloaddition.

In some embodiments, a method of preparing an activated fluorophore-conjugated fucose may further include purifying and/or concentrating the activated fluorophore-conjugated fucose.

In some embodiments, the GDP-f-Fuc may be prepared as described in Example 2.

### Methods of Using a Fluorophore-Conjugated Fucose

This disclosure further describes methods for using a fluorophore-conjugated fucose preferably, an activated fluorophore-conjugated fucose including, for example, GDP-f-Fuc.

In some embodiments, an activated fluorophore-conjugated fucose (for example, GDP-f-Fuc) is used in a method for specific glycan labeling (also referred to herein as Direct Fluorescent Glycan Labeling (DFGL)), in which fluorophore-conjugated fucose is directly attached to target glycans via specific fucosyltransferases. Some exemplary methods are shown in FIG. 8 and described in Example 2 and Example 3.

As described above and shown in Example 1, a direct fluorescent glycan labeling (DFGL) strategy was first developed to label and detect the substrate glycans of various sialyltransferases. Surprisingly, as described below and shown in Example 2, DFGL may also be used to label and detect the substrate glycans of some fucosyltransferases.

First, it was unknown whether fucosyltransferases would tolerate and incorporate a fluorophore-conjugated fucose. And, indeed, incorporation of a fluorophore-conjugated glucose or galactose by the relevant enzymes were so slow as to be unusable. For example, incorporation of a fluorophore-conjugated glucose was more than 1000 times less efficient than incorporation of a fluorophore-conjugated fucose by their respective glycosyltransferases. Similarly, fluorophore-conjugated glucosamine and fluorophore-conjugated galactosamine were not successfully incorporated using DFGL.

Moreover, for fucosyltransferases—unlike for sialyltransferases, as shown in FIG. 8, panels D and E, and described in Example 2, to detect high-mannose glycans or probe core fucosylation modification of a glycan requires different pre-treatment of the glycan. For example, to detect a high-mannose glycan by incorporation of a fluorophore-conjugated fucose using FUT8, terminal α-2 linked mannose residues must be removed using mannoisidase, then a GlcNAc residue must be added using MGAT1 before the glycan is exposed to GDP-f-Fuc and FUT8 (see FIG. 8, panel D). In another example, to probe the status of core fucosylation, the terminal sialic acid residues must be removed using neuraminidase, then the galactose residues removed using galactosidase before the glycan is exposed to GDP-f-Fuc and FUT8 (see FIG. 8, panel E).

In some embodiments, the method includes mixing a target glycan with an activated fluorophore-conjugated fucose (for example, GDP-f-Fuc), and a fucosyltransferase. In some embodiments the target glycan, the activated fluorophore-conjugated fucose, and the fucosyltransferase may be mixed in a buffer.

In some embodiments, the method includes mixing a target protein (for example, a glycoprotein) which includes a glycan with an activated fluorophore-conjugated fucose (for example, GDP-f-Fuc), and a fucosyltransferase. In some embodiments the target protein, the activated fluorophore-conjugated fucose, and the fucosyltransferase may be mixed in a buffer.

In an exemplary embodiment, the buffer includes 25 mM Tris pH 7.5 and 10 mM MnCl₂.

The method may include using any fucosyltransferase that is able to incorporate the fluorophore-conjugated fucose into a glycan. In some embodiments, the fucosyltransferase may include, for example, FUT1, FUT2, FUT3, FUT4, FUT5, FUT6, FUT7, FUT8, FUT9, FUT10, or FUT11, or a combination thereof. In some embodiments, the fucosyltransferase may include, for example, FUT2, FUT6, FUT7, FUT8, and FUT9, or a combination thereof. In some embodiments, the fucosyltransferase preferably includes a human fucosyltransferase. In some embodiments, the fucosyltransferase includes a non-human fucosyltransferase. As described in Example 2, fluorophore-conjugated fucose was incorporated using FUT2, FUT6, FUT7, FUT8, and FUT9.

In some embodiments, the fucosyltransferase may be selected based on the type of fucosylation to be detected. For example, FUT6 may incorporate a fluorophore-conjugated fucose to both lactosamine (LN) and sialyl-lactosamine (sLN), and would not be selected if the intent was to distinguish LN and sLN. In contrast, the more specific FUT9 and FUT7 might be used to detect LN and sLN, respectively.

In some embodiments, the target glycan or the target protein, the activated fluorophore-conjugated fucose, and the fucosyltransferase are incubated together for at least 1 minute, at least 5 minutes, at least 10 minutes, at least 15 minutes, at least 20 minutes, at least 25 minutes, or at least 30 minutes. In some embodiments, the target glycan or the target protein, the activated fluorophore-conjugated fucose, and the fucosyltransferase are incubated together for up to 10 minutes, up to 15 minutes, up to 20 minutes, up to 25 minutes, up to 30 minutes, up to 1 hour, up to 2 hours, up to 24 hours, or up to 48 hours. In an exemplary embodiment, the target protein, the activated fluorophore-conjugated fucose, and the fucosyltransferase are incubated together for 30 minutes.

The target glycan or the target protein, the activated fluorophore-conjugated fucose, and the fucosyltransferase may be incubated together at any temperature at which the fucosyltransferase is able to incorporate the fluorophore-conjugated fucose into the target glycan or into a glycan of the target protein. In some embodiments, the target glycan or the target protein, the activated fluorophore-conjugated fucose, and the fucosyltransferase may be incubated together at a temperature of at least 20° C., at least 25° C., at least 28° C., or at least 30° C. In some embodiments, the target glycan or the target protein, the activated fluorophore-conjugated fucose, and the fucosyltransferase may be incubated together at a temperature of up to 32° C., up 35° C., up 37° C., up to 40° C., up to 45° C., or up to 50° C. In an exemplary embodiment, the target glycan or the target protein, the activated fluorophore-conjugated fucose, and the fucosyltransferase may be incubated together at 37° C.

In some embodiments, the target glycan or the target protein may be pre-treated prior to being mixed with the activated fluorophore-conjugated fucose and the fucosyltransferase. For example, to detect high-mannose glycans or to probe for core fucosylation modification of a glycan of the target protein, pre-treatment of the protein may be required to remove or to add some terminal sugar residues on a glycan or glycans of the target protein.

In some embodiments, to probe for a core fucosylation modification, the target glycan or the target protein may be pre-treated with neuraminidase or galactosidase, or both. In some embodiments, the target glycan or the target protein may be pre-treated with neuraminidase and then galactosidase before being mixed with activated fluorophore-conjugated fucose (for example, GDP-f-Fuc) and the fucosyltransferase. In some embodiments, the target glycan or the target protein may be pre-treated in the presence of UDP-GlcNAc.

In some embodiments, to detect a high-mannose glycan, the target glycan or the target protein may be pre-treated with α-2 specific mannosidase, or MGAT1, or both. In some embodiments, the target protein may be pre-treated with α-2 specific mannosidase and MGAT1 before being mixed with activated fluorophore-conjugated fucose (for example, GDP-f-Fuc) and the fucosyltransferase. In some embodiments, the target glycan or the target protein may be pre-treated in the presence of UDP-GlcNAc.

In some embodiments, the method includes separating the mixture including the target glycan or the target protein, the fluorophore-conjugated fucose, and the fucosyltransferase. In some embodiments, the components of the mixture may be separated using protein gel electrophoresis including, for example, sodium dodecyl sulfate—polyacrylamide gel electrophoresis (SDS-PAGE), capillary gel electrophoresis, isoelectric focusing electrophoresis, etc.

In some embodiments, as exemplified in Example 4, and as further discussed below, prior to separating the components, the method may further include cleaving a glycan including the fluorophore-conjugated fucose from the target protein. In such embodiments, as further discussed below, the labeled glycans cleaved from the target protein may be separated to assess mobility of the labeled glycans.

In some embodiments, the method includes imaging the separated components. For example, in an exemplary embodiment, when the components are incorporated in an SDS-PAGE gel, they may be imaged by fluorescent imaging for example, using a fluorescent imager. Additionally or alternatively, the components may be imaged using trichloroethanol (TCE) staining, silver staining, or Coomassie blue staining, or a combination of these methods. That is, in contrast to previous methods which relied on Western blotting to visualize the components, a combination of imaging methods may be used.

When the glycans have not been cleaved from a target protein, separating the mixture and then imaging the separated components allows assessment of the labeling of the target protein. This process is also referred to herein as the Direct Fluorescent Glycan Labeling (DFGL) method.

When the glycans have been cleaved from a target protein, separating the mixture and imaging the labeled glycans allows for characterization of a glycan or of the glycosylation of a glycoprotein by assessing their mobility, as further described below.

As described in Example 2, the substrate glycans of a fucosyltransferase may be detected via enzymatic incorporation of a fluorophore-conjugated fucose by a fucosyltransferase including FUT2, FUT6, FUT7, FUT8, and FUT9. Example 2 further describes the detection of substrate glycans of FUT8 and FUT9 on therapeutic antibodies and the detection of high mannose glycans on glycoproteins by enzymatic conversion of high mannose glycans to the substrate glycans of FUT8. By establishing a series of precursor glycans, the substrate specificities of FUT8 were also demonstrated.

In some embodiments, DGFL may be effective on a wide variety of glycoproteins. Exemplary glycoproteins include fetal bovine fetuin (Ma et al. Glycobiology 16, 158R-184R (2006)), which contains complex N-glycans and O-glycans; ribonuclease B (Prien et al. J Am Soc Mass Spectrom 20, 539-556 (2009)), which contains high mannose N-glycans; insect cell expressed recombinant H1N1 neuraminidase (Wu et al. Biochem Biophys Res Commun 473, 524-529 (2016)), which contains Man3 type high mannose N-glycan; and Cantuzumab (Rodon et al. Cancer Chemother Pharmacol 62, 911-919 (2008)), a therapeutic antibody that contains complex type of N-glycans.

As described in Example 2, using enzymatic incorporation of a fluorophore-conjugated fucose, the presence of the substrate glycans of various fucosyltransferases on glycoproteins, particularly on therapeutic antibodies, may be revealed. The detection of high mannose glycans on glycoproteins and the substrate specificities of FUT8 were also demonstrated.

### Methods of Using Fluorophore-Conjugated Sialic Acid and Fluorophore-Conjugated Fucose

In another aspect, this disclosure further describes methods for using a fluorophore-conjugated sialic acid and a fluorophore-conjugated fucose.

Using simultaneous enzymatic incorporation of both a fluorophore-conjugated sialic acid and a fluorophore-conjugated fucose, the interplay between fucosylation and sialylation may be studied or demonstrated.

In some embodiments, a target glycan or a target protein (for example, a glycoprotein) may be simultaneously labeled with a sialic acid and a fucose that are conjugated to different types of fluorophores. Additionally or alternatively, a target glycan or a target protein may be labeled with a fluorophore-conjugated sialic acid and then labeled with a fluorophore-conjugated fucose or labeled with a fluorophore-conjugated fucose and then a fluorophore-conjugated sialic acid.

In some embodiments, a target glycan or a target protein may be exposed to a sialyltransferase and a fucosyltransferase at the same time. Additionally or alternatively, a target glycan or a target protein may be exposed to a fucosyltransferase and then a sialyltransferase or exposed to a sialyltransferase and then a fucosyltransferase.

In some embodiments, the method may further include adding a C. perfringens neuraminidase to the mixture including the target glycan or the target protein, the fluorophore-conjugated sugar, and the sialyltransferase and/or fucosyltransferase.

In some embodiments, the target glycan or the target protein may be pre-treated prior to being mixed with the fluorophore-conjugated fucose and/or the fucosyltransferase. In some embodiments, the target glycan or the target protein may be pre-treated prior to being mixed with the fluorophore-conjugated sialic acid (including, for example, CMP-f-SA) and/or the sialyltransferase.

For example, as described in Example 2, using enzymatic incorporation of fluorophore-conjugated fucoses and sialic acids, dual labeling of N- and O-glycans on the cellular extracts of HEK293 cells and the interplay between FUT9 and N-glycan specific sialyltransferase ST6Gal1 were demonstrated.

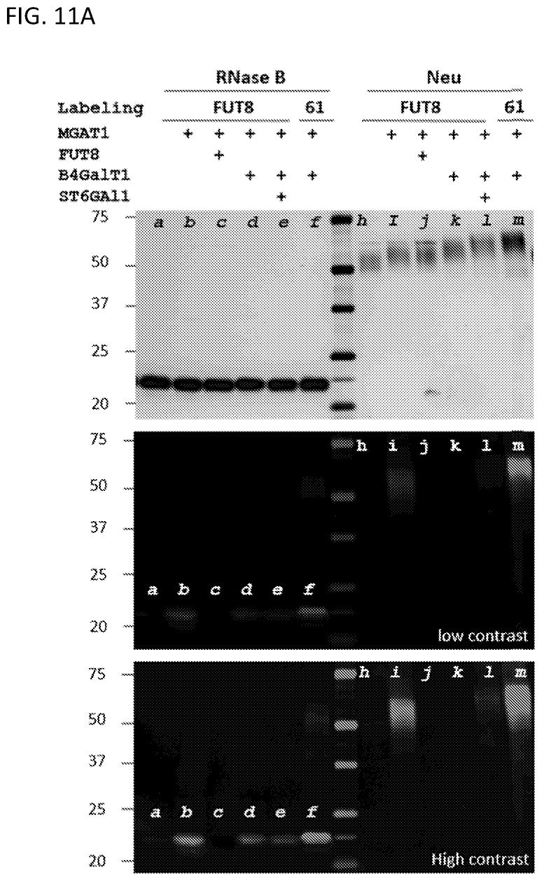

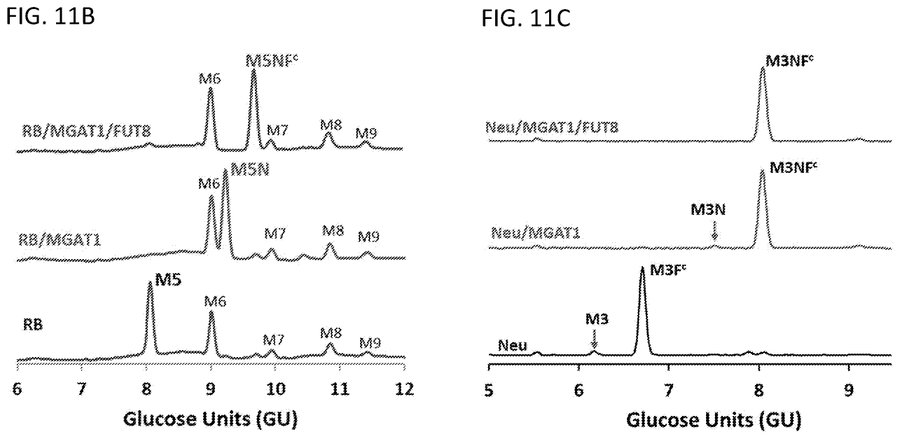

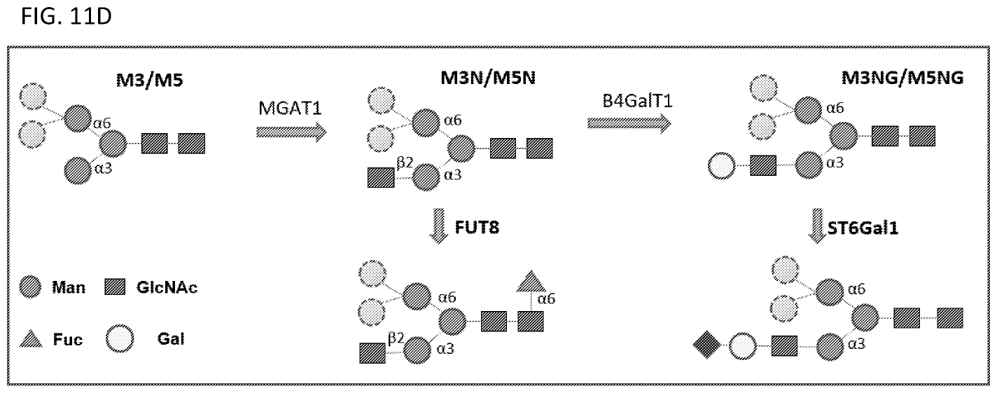

In some cases, glycans may be labeled by either a sialyltransferase or a fucosyltransferase. For example, terminal lactosamine may be labeled by either ST6Gal1 or FUT9, and high-mannose glycans may be converted to substrate glycan for either ST6Gal1 or FUT8 for labeling (FIG. 11). In other cases, however, a substrate glycan may only be revealed by incorporation of a fluorophore-conjugated sialic acid or a fluorophore-conjugated fucose. For example, the status of core-6 fucosylation may only be revealed by fluorophore-conjugated fucose incorporation by FUT8, and the sialylation on core-1 O-glycan may only be revealed by fluorophore-conjugated sialic acid incorporation by ST3Gal1 or ST3Gal2.

The interplay between sialylation and fucosylation may determine some important biological properties of a cell. For example, the counteractive action between FUT9 and ST3Gal4 determines the sensitivity of a cell to the toxin ricin (Taubenschmid et al. Cell Res 27, 1351-1364 (2017)), and the counteractive action between FUT2 and ST3Gals determines the expression of sialyl Lewis X expression (Zerfaoui et al. Eur J Biochem 267, 53-61 (2000)). By labeling with both fluorophore-conjugated sialic acid and/or a fluorophore-conjugated fucose, this interplay may be studied. As an example, in Example 2, the mutually exclusive relationship between the sialylation by ST6Gal1 and the fucosylation by FUT9 was demonstrated. Since sialylation by ST6Gal1 creates the receptors for H1N1 influenza virus (Viswanathan et al. Glycoconj J 27, 561-570 (2010)), the counteractive action by FUT9 could mitigate the susceptibility of a cell to the virus. In contrast, no obvious interplay was found between ST3Gal2 and FUT9, which is expected because these two enzymes recognize different substrate glycans.

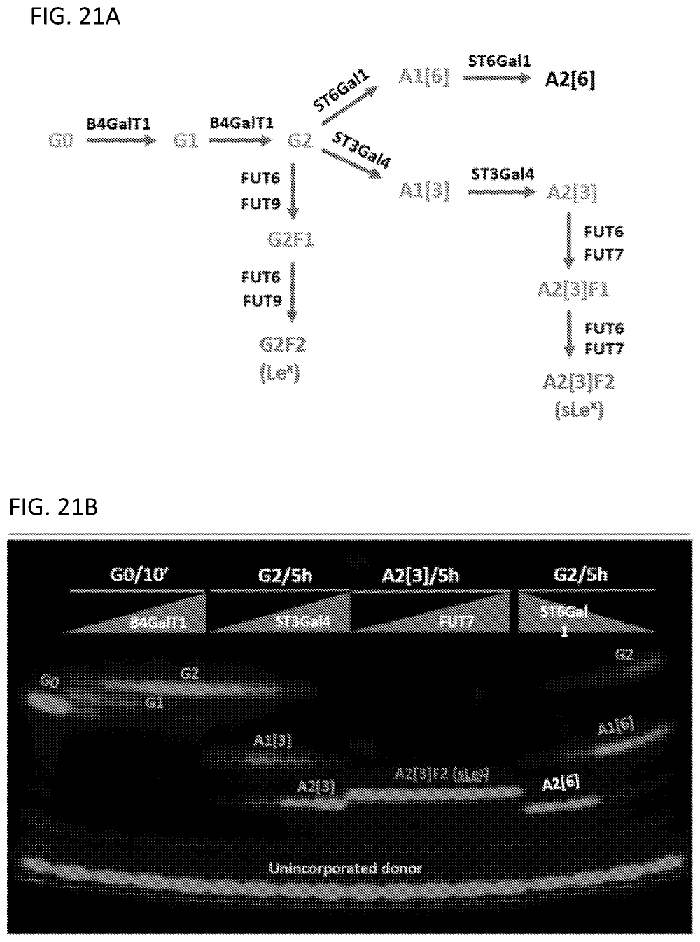

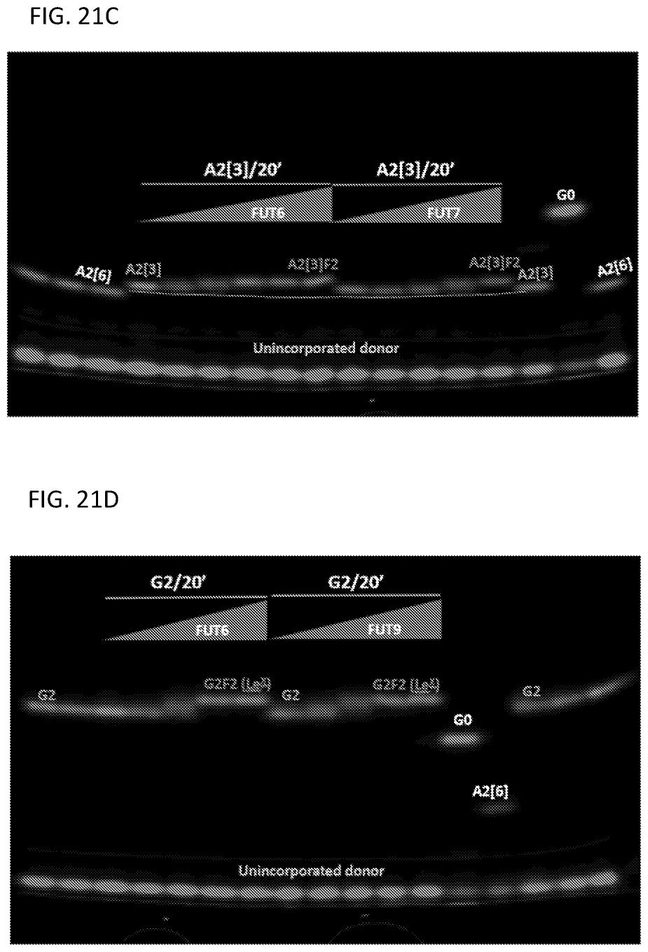

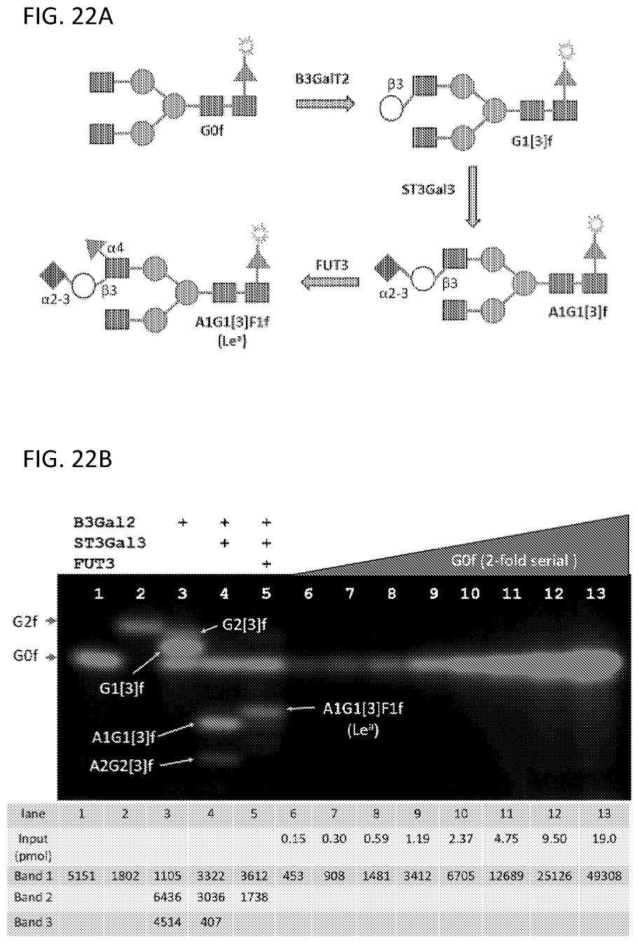

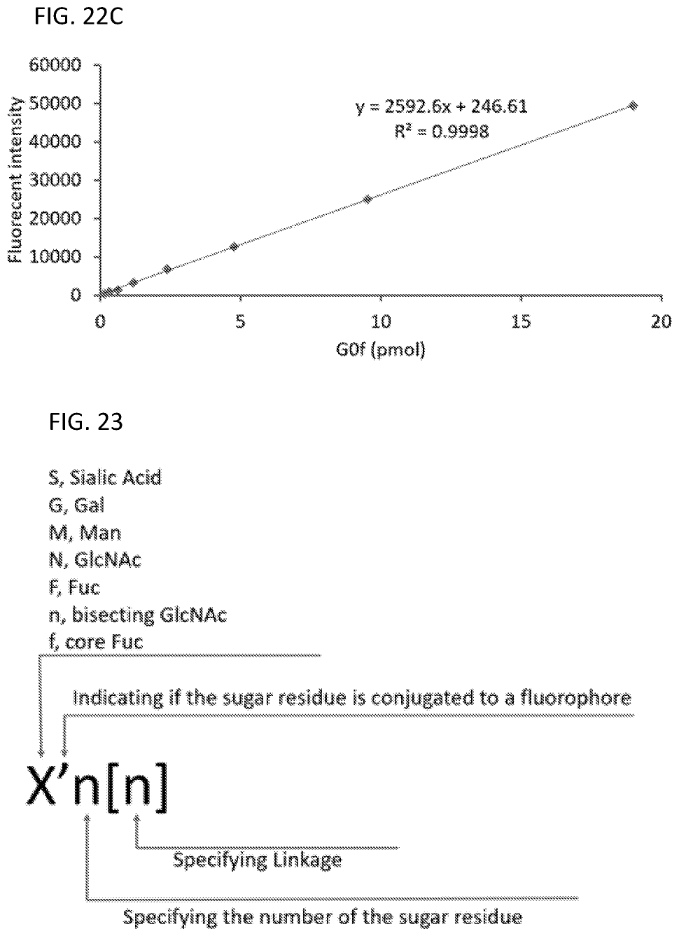

In some embodiments, a fluorophore-conjugated sialic acid or a fluorophore-conjugated fucose may be used to elucidate glycan synthesis. Glycan synthesis is determined by the availability of individual glycosyltransferases and their substrate glycans, therefore characterizing these glycosyltransferases and their kinetics is key to the understanding of how glycan epitopes are synthesized. As shown in Example 3, using fluorophore-conjugated fucose may be particularly helpful for studying these enzymatic processes. For example, the enzymatic synthesis of Le^{x} and sLe^{x} is exemplified in FIG. 21 and the enzymatic synthesis of Le^{a} and sLe^{a} is exemplified in FIG. 22.

### Methods of Using DFGL and Glycan Mobility to Characterize the Glycosylation of a Glycan or Glycoprotein

In some embodiments, the methods described herein may be used to characterize a glycan or to characterize the glycosylation of a glycoprotein. For example, the methods may involve labeling a glycan and then detecting the mobility of the resulting glycan. As described in Examples 3 and 4, the overall glycosylation pattern of a protein may also be determined using this method, allowing for screening of a protein for consistent glycosylation and/or abnormal glycosylation.

In some embodiments, a target glycan may be labeled with a fluorophore-conjugated sugar, forming a labeled glycan. In some embodiments, the target glycan may be labeled with a fluorophore-conjugated sialic acid or a fluorophore-conjugated fucose, or both.

In some embodiments, at the time of incorporation of the fluorophore-conjugated sugar into the target glycan, the target glycan may be present on a target protein. That is, a target glycan on a target protein may be labeled with a fluorophore-conjugated sugar, resulting in a labeled target protein.

In some embodiments, the mobility of a labeled glycan (that has never been attached to a target protein) may be evaluated. In some embodiments, the mobility of the labeled target protein may be evaluated. Additionally or alternatively, a labeled glycan may be cleaved from the labeled target protein to form a freed labeled glycan. The mobility of the freed labeled glycan may be evaluated.

A labeled glycan may be cleaved from the target protein by any suitable method. The cleavage may preferably be enzymatic. For example, the glycan may be cleaved from the target protein by an endoglycosidase. Exemplary endoglycosidases include PNGaseF, Endo F1, Endo F2, Endo F3, Endo M and EndoS.

In some embodiments, the mobility of the labeled glycan, the labeled target protein, or the free labeled glycan may be measured using electrophoresis. Any suitable means of electrophoresis may be used including, for example, sodium dodecyl sulfate—polyacrylamide gel electrophoresis (SDS-PAGE), capillary electrophoresis. In some embodiments, SDS-PAGE may be preferred. Chromatographic separation techniques, such as ion-exchange chromatography and paper chromatography, may also be useful.

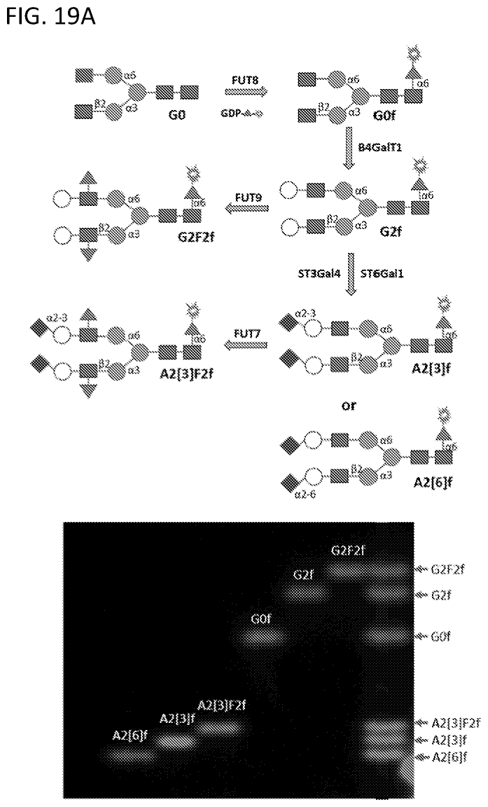

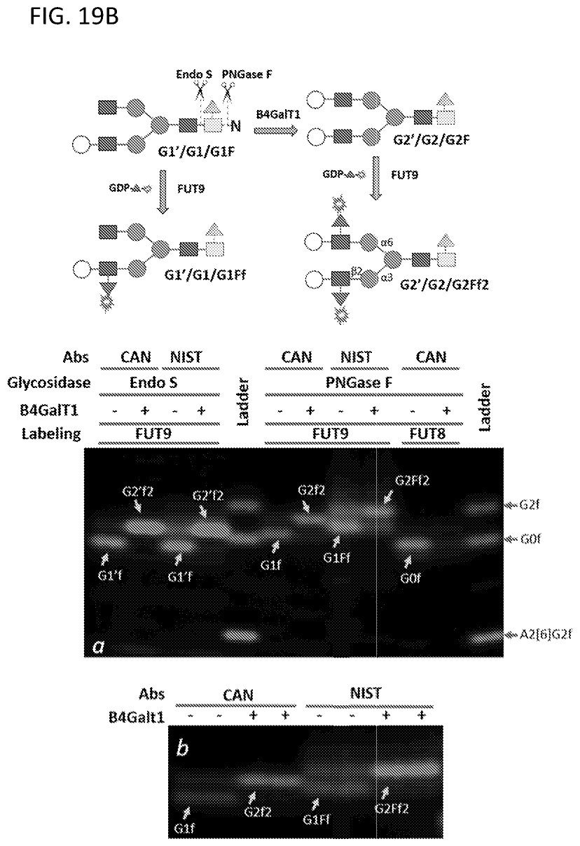

Example 3 shows an example of how electrophoresis in combination with DFGL may be applied to study glycans. During electrophoresis, separation is based on the differences on charge, mass and molecular structures of the glycans. Charges may be introduced along with the incorporation of fluorophores that are usually negatively charged. Sialic acids naturally contribute negative charges, therefore, sialylated glycans have much faster mobility than other glycans. Neutral sugars such as galactose and fucose usually slow down the mobility of a glycan. Glycans with same charge and mass may be separated on structural differences. For examples, while monogalactosylated G1[3] and G1[6] were not separated in FIG. 19B, di-sialylated A2[3] and A2[6] were well separated in FIG. 19A.

In some embodiments, the method may further include visualizing the labeled glycan, the freed labeled glycan, or the labeled target protein. If gel electrophoresis has been used, the labeled glycan, the freed labeled glycan, or the labeled target protein may be visualized while still present in a gel. Any suitable method of measuring the labeled glycan may be used including, for example, by detecting fluorescence (UV, infrared, or visible), measuring chemiluminescence, silver staining, trichloroethanol (TCE) staining, etc.

In some embodiments, measuring the mobility of the glycan may include comparing the mobility of the labeled glycan or the freed labeled glycan to the mobility of a glycan standard or a glycan ladder. As further described below, a glycan standard includes a single labeled (for example, fluorophore-conjugated) glycan of known structure. A glycan ladder includes multiple (that is, at least two) glycan standards.

In some embodiments, a glycan ladder may include each of the labeled glycans included in a reaction. In some embodiments, the glycan ladder may preferably include labeled versions of each of the possible glycans present in a sample.

In some embodiments, comparing the mobility of labeled glycan or a freed labeled glycan to the mobility of the reference glycans (alone or in a glycan ladder) may allow for the determination of the identity of a labeled band including a labeled glycan or a freed labeled glycan.

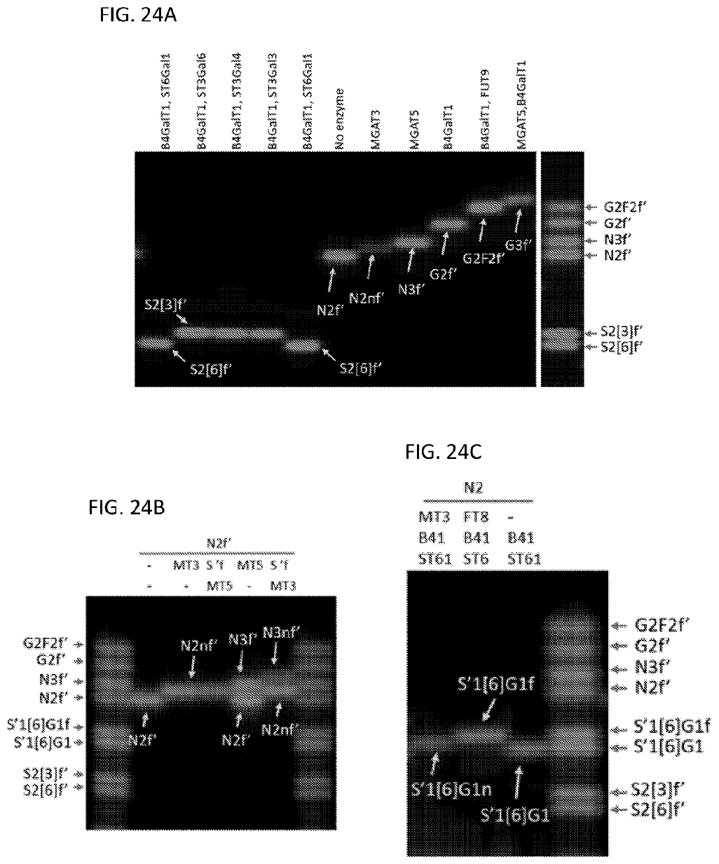

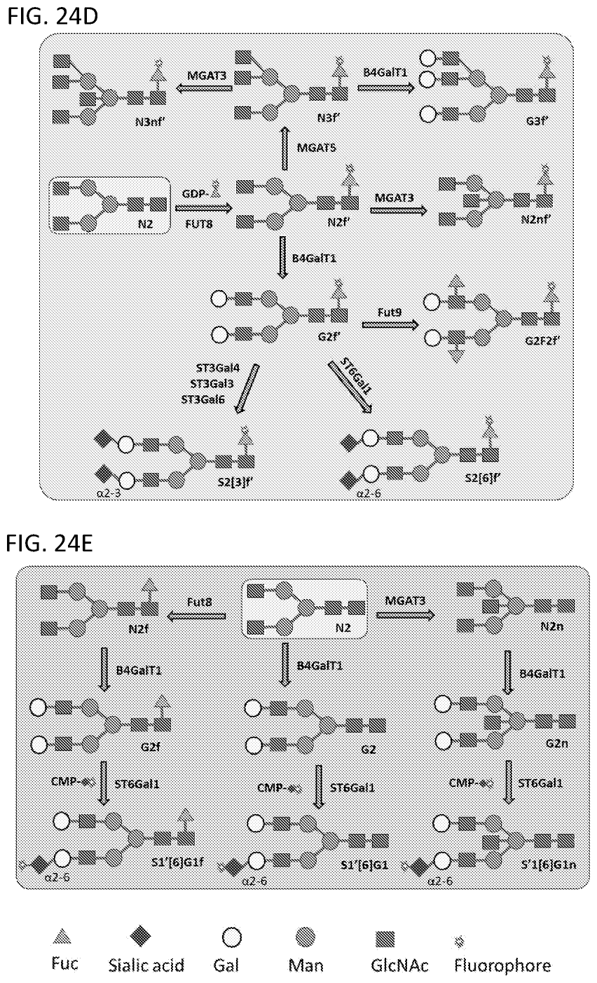

As further described in Example 4, the addition of a linkage specific monosaccharide may change the mobility of a glycan at relatively constant rate (FIG. 24). Information about the mobility shift caused by the addition of certain monosaccharides may also allow for the deduction of the identities of labeled bands.

In some embodiments, the methods described herein may be used to detect the glycosylation of antibodies. As described in, for example, Cobb, The History of IgG Glycosylation and Where We Are Now, Glycobiology 2019, glycosylation affects antibody structure and function.

For example, in some embodiments, the methods described herein may be used to characterize the glycosylation of monoclonal antibody drugs. For example, the methods described herein may be used to characterize the glycosylation of monoclonal antibodies during antibody production.

For example, the sialylation of the glycans on an antibody may be assayed using ST6Gal1 or FUT9; the existence of high-mannose glycans on an antibody may be assayed using the FUT8 in combination with α-2 mannosidase and MGAT1; the status of core-6 fucosylation of the glycans on an antibody may be probed by using a combination of neuraminidase and β-galactosidase.

At the time of the invention, glycosylation analysis was achieved mainly through mass spectrometry analysis, which requires expensive instrumentation and highly trained personnel expertise. In contrast, the methods described herein and exemplified in Example 4 allow for glycosylation analysis (also referred to as glycan fingerprinting) based on enzymatic fluorescent glycan labeling and electrophoresis. These methods may provide a quick and inexpensive way to interrogate if different batches of glycoproteins exhibit consistent glycosylation and/or to screen for samples including abnormal glycosylation. Although the strategy does not allow site specific and detailed structural glycan analysis, these methods offer some major advantages over mass spectrometry analysis. First, the method is simple, convenient, and much more affordable. Second, the data acquired are visually informative and therefore rather easy to interpret. Third, multiple samples can be processed simultaneously, therefore it is highly efficient. Fourth, the signal intensity is directly related to the abundance of a glycan species and therefore is more quantitative (Wu et al. Glycobiology, cwaa030 (2020)). While this method only reveals the substrate glycans of the labeling enzyme, and glycans that are not recognized by the labeling enzyme remain to be undetected, this information could be advantageous when only specific glycans are being examined.

### Glycan Ladder

In another aspect, this disclosure describes a glycan ladder. A glycan ladder includes at least two labeled (e.g., fluorophore-conjugated) glycans. In some embodiments, the labeled glycans may include a fluorophore-conjugated sialic acid or a fluorophore-conjugated fucose or both. The identity of the labeled glycans in the glycan ladder is preferably known.

In some embodiments, the glycan ladder may include equal amounts of each labeled glycan.

In some embodiments, a glycan ladder may include each of the labeled glycans included in a reaction. In some embodiments, the glycan ladder may preferably include labeled versions of each of the possible glycans present in a sample.

The glycans of the glycan ladder may be labeled with any suitable label. The label is preferably included by incorporation of a labeled sugar (for example, a fluorescent sialic acid or a fluorescent fucose) into the target glycan. Exemplary labels include fluorophores, biotin, radioactive isotopes, etc. In some embodiments, the label is preferably a fluorophore. In some embodiments, the fluorophore may preferably have an emission wavelength in the visible spectrum (that is, about 380 nm to about 740 nm). Exemplary fluorophores include the fluorophores of the Alexa Fluor® family such as Alexa Fluor® 488 and Alexa Fluor® 555. Additional exemplary fluorophores include Cy5 and Cy3. Combinations of fluorophores may also be useful.

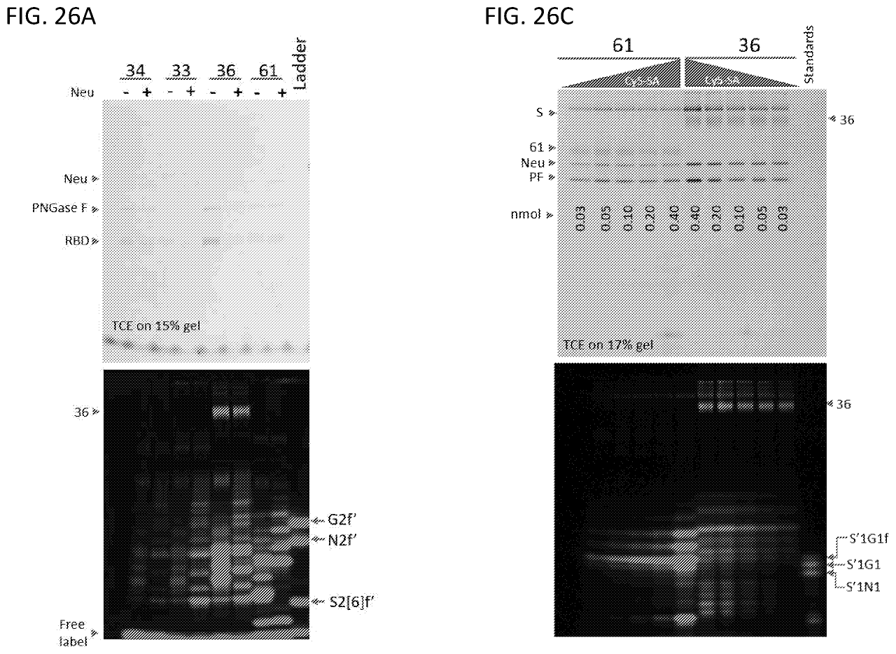

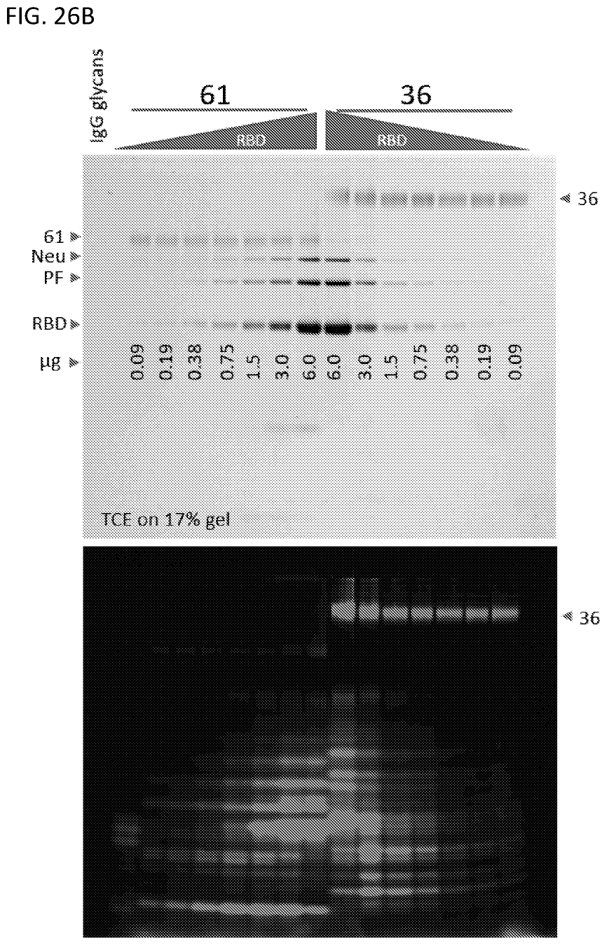

In some embodiments, a glycan ladder may include a mixture of extended labeled glycans. An extended glycan is formed from a glycan extended by a glycotransferase. An extended labeled glycan is formed from a labeled glycan extended by a glycotransferase. In some embodiments, the extended labeled glycans may each be formed from the same labeled glycan using a variety of glycotransferases. For example, the glycan ladders shown in FIG. 26A (including G2f′, N2f′, and S2[6]f′) were generated from a single labeled glycan N₂f. In some embodiments, the extended labeled glycans may be formed from a combination of two or more labeled glycans. For example, the glycan ladder of FIG. 24B and FIG. 24C (including G2F2f′, G2f′, N3f′, N2f′, S′1[6]G1f, S′1[6]G1, S2[3]f′, and S2[6]f′) are derived from two labeled glycans that were labeled through fluorescent sialic acid and fucose (specifically, G2F2f′, G2f′, N3f′, S2[3]f′, and S2[6]f′ were extended from N2f′, S′1[6]G1f and S′1[6]G1 were generated by addition of a fluorescent sialic acid to G1f and G1, respectively. (The nomenclature of the glycans in the previous sentence is as described in FIG. 23 and its legend).

An exemplary embodiment is described in Example 4, where Cy5-Fucose labeled glycan was extended by a variety of glycosyltransferases (including MGAT3, MGAT5, B4GalT1, FUT9, ST3Gal6), and a glycan ladder was built by mixing equal amounts of the extended labeled glycans.

In some embodiments, the glycan ladder includes at least three labeled (for example, fluorophore-conjugated) glycans, at least four labeled glycans, at least five labeled glycans, at least six labeled glycans (see, for example, FIG. 19A and FIG. 24A), at least seven labeled glycans, or at least eight labeled glycans (see, for example, FIG. 24B and FIG. 24C).

In some embodiments, the glycans of the glycan ladder may be selected for a specific purpose. For example, in some embodiments, the glycans of the glycan ladder may be selected to characterize glycans from an unknown antibody. Exemplary combinations of glycans that might be useful for the characterization of glycans from an unknown antibody include combinations of two or more of G2F2f′, G2f′, N3f′, N2f′, S′1[6]G1f, S′1[6]G1, S2[3]f′, and S2[6]f′ (wherein the nomenclature of these glycans is as described in FIG. 23 and its legend). For example, the glycan ladder used in FIG. 24A included G2F2f′, G2f′, N3f′, N2f′, S2[3]f′, and S2[6]f′ (wherein the nomenclature of these glycans is as described in FIG. 23 and its legend). In yet another example, the glycan ladders in FIG. 24B and FIG. 24C included G2F2f′, G2f′, N3f′, N2f′, S′1[6]G1f, S′1[6]G1, S2[3]f′, and S2[6]f′ (wherein the nomenclature of these glycans is as described in FIG. 23 and its legend). Combinations and sub-combinations of these specific examples are also envisioned.

In some embodiments, a composition including the glycan standard or the glycan ladder may be suitable for use in an assay to evaluate the mobility of a glycan. For example, the composition may be suitable for use in an electrophoresis assay. In some embodiments the composition may include a buffer compound. Exemplary buffer compounds include Tris, HEPES, etc.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Fluorophore-Conjugated Sialic Acid Aspects

Aspect A1 is composition comprising a fluorophore-conjugated sialic acid.

Aspect A2 is the composition of Aspect A1, wherein the fluorophore-conjugated sialic acid comprises an activated fluorophore-conjugated sialic acid.

Aspect A3 is the composition of Aspect A1 or A2, wherein the fluorophore-conjugated sialic acid comprises a cytidine monophosphate activated fluorophore-conjugated sialic acid (CMP-f-SA). Aspect A4 is the composition of any one of the previous Aspects, wherein the fluorophore-conjugated sialic acid comprises N-acetyl-neuraminic acid (Neu5Ac or NANA), 2-keto-3-deoxynononic acid (Kdn), N-glycolylneuraminic acid (Neu5Gc), neuraminic acid (Neu), or 2-deoxy-2,3-didehydro-Neu5Ac (Neu2en5Ac), or a combination thereof.

Aspect A5 is the composition of any one of the previous Aspects, wherein the fluorophore-conjugated sialic acid comprises Alexa Fluor® 488, Alexa Fluor® 555, or Cy5.

### Exemplary Methods of Making Fluorophore-Conjugated Sialic Acid Aspects

Aspect B1 is a method comprising incubating a CMP-Azido-Sialic acid (CMP-N₃-SA) and an alkyne-conjugated fluorophore.

Aspect B2 is the method of Aspect B1, wherein the CMP-N₃-SA and the alkyne-conjugated fluorophore are conjugated via copper (I)-catalyzed azide-alkyne cycloaddition.

Aspect B3 is the method of Aspect B1 or B2, wherein the method further comprises forming cytidine monophosphate activated fluorophore-conjugated sialic acid (CMP-f-SA).

Aspect B4 is the method of Aspect B3, wherein the method further comprises purifying the CMP-f-SA.

Aspect B5 is the method of Aspect B3 or Aspect B4, wherein the method further comprises concentrating the CMP-f-SA.

### Exemplary Methods of Using Fluorophore-Conjugated Sialic Acid Aspects

Aspect C1 is a method comprising using a fluorophore-conjugated sialic acid wherein the method comprises attaching the fluorophore-conjugated sialic acid to a glycan to form a labeled glycan.

Aspect C2 is the method of Aspect 1, wherein the fluorophore-conjugated sialic acid comprises the fluorophore-conjugated sialic acid of any one of the Exemplary Fluorophore-Conjugated Sialic Acid Aspects (A1-A5).

Aspect C3 is the method of Aspect 1 or Aspect 2, wherein the method comprises attaching the fluorophore-conjugated sialic acid to the glycan using a sialyltransferase.

Aspect C4 is the method of Aspect 3, wherein the sialyltransferase comprises ST3Gal1, ST3Gal2, ST3Gal3, ST3Gal4, ST3Gal5, ST3Gal6, ST6Gal 1, ST6Gal2, ST6GalNAc1, ST6GalNAc2, ST6GalNAc3, ST6GalNAc4, ST6GalNAc5, ST6GalNAc6, ST8SIA1, ST8SIA2, ST8SIA3, ST8SIA4, ST8SIA5, or ST8SIA6, or a combination thereof.

Aspect C5 is the method of any one Aspects C1 to C4 wherein the method comprises mixing a target protein comprising the glycan with the fluorophore-conjugated sialic acid, and a sialyltransferase.

Aspect C6 is the method of any one Aspects C1 to C5, wherein the method comprises mixing the glycan, the fluorophore-conjugated sialic acid, and the sialyltransferase in a buffer.

Aspect C7 is the method of any one Aspects C1 to C6, wherein the method comprises incubating the glycan, the fluorophore-conjugated sialic acid, and the sialyltransferase together for at least 1 minute and up to 48 hours.

Aspect C8 is the method of any one Aspects C1 to C7 wherein the glycan, the fluorophore-conjugated sialic acid, and the sialyltransferase are incubated together at a temperature of at least 20° C. and up to 50° C.

Aspect C9 is the method of any one of Aspects C1 to C8, wherein the method comprises mixing the glycan, the fluorophore-conjugated sialic acid, and the sialyltransferase with a C. perfringens neuraminidase.

Aspect C10 is the method of any one of Aspects C5 to C9, wherein the method comprises attaching the fluorophore-conjugated sialic acid to the glycan on the target protein to form a labeled target protein comprising the labeled glycan.

Aspect C11 is the method of any one of Aspects C1 to C10, wherein the method further comprises separating components of a mixture comprising the labeled glycan or the labeled target protein comprising the labeled glycan.

Aspect C12 is the method of Aspect C11, wherein separating the components comprises gel electrophoresis of a composition comprising the labeled glycan or a composition comprising the labeled protein.

Aspect C13 is the method of Aspect C11 or C12, wherein the method comprises imaging the labeled glycan or labeled target protein.

Aspect C14 is the method of any one of Aspects C1 to C13, wherein imaging the labeled glycan or the labeled target protein, the fluorophore-conjugated sialic acid, and/or the sialyltransferase comprises using silver staining, trichloroethanol (TCE) staining, fluorescent imaging, or a combination thereof.

Aspect C15 is the method of any one of Aspects C10 to C14, wherein the method comprises cleaving the labeled glycan from the labeled target protein to form a freed labeled glycan.

Aspect C16 is the method of Aspect C16, wherein the method comprises comparing mobility of the freed labeled glycan to mobility of a glycan standard or a glycan ladder, wherein the glycan standard comprises a fluorophore-conjugated glycan and wherein the glycan ladder comprises two or more fluorophore-conjugated glycans.

Aspect C17 is the method of Aspect C16, wherein the glycan ladder comprises a combination of extended glycans.

Aspect C18 is the method of Aspect C17, wherein the extended glycans comprise extended labeled glycans, and wherein the extended labeled glycans comprise fluorophore-conjugated glycans.

Aspect C19 is the method of Aspect C15 or C16, wherein the extended glycans comprise a glycan or glycans extended by one or more glycosyltransferases.

Aspect C20 is the method of C19, wherein the one or more glycosyltransferases comprise MGAT3, MGAT5, B4GalT1, FUT9, or ST3Gal6, or a combination thereof.

Aspect C21 is the method of any one of Aspects C15 to C20, wherein the method comprises gel electrophoresis of a composition comprising the free labeled glycan and imaging the freed labeled glycan.

Aspect C22 is the method of any one of Aspects C1 to 21, wherein the method further comprises attaching a fluorophore-conjugated fucose to a glycan.

Aspect C23 is the method of any one of Aspects C5 to C22, wherein the target protein comprises a monoclonal antibody or a viral protein.

### Exemplary Fluorophore-Conjugated Fucose Aspects

Aspect D1 is a composition comprising a fluorophore-conjugated fucose.

Aspect D2 is the composition of Aspect D1, wherein the fluorophore-conjugated fucose comprises guanosine diphosphate activated fluorophore-conjugated fucose (GDP-f-Fuc).

Aspect D3 is the composition of Aspect D1 or D2, wherein the fluorophore-conjugated fucose comprises Alexa Fluor® 488, Alexa Fluor® 555, or Cy5.

### Exemplary Methods of Making Fluorophore-Conjugated Fucose Aspects

Aspect E1 is a method comprising incubating a GDP-Azido-Fucose (GDP-N₃-Fucose) and an alkyne-conjugated fluorophore.

Aspect E2 is the method of Aspect E1, wherein the GDP-N₃-Fucose and the alkyne-conjugated fluorophore are conjugated via copper (1)-catalyzed azide-alkyne cycloaddition.

Aspect E3 is the method of Aspect E1 or E2, wherein the method further comprises forming guanosine diphosphate activated fluorophore-conjugated fucose (GDP-f-Fuc).

Aspect E4 is the method of Aspect E3, wherein the method further comprises purifying the GDP-f-Fuc.

Aspect E5 is the method of Aspect E3 or E4, wherein the method further comprises concentrating the GDP-f-Fuc.

### Exemplary Methods of Using Fluorophore-Conjugated Fucose Aspects

Aspect F1 is a method comprising using a fluorophore-conjugated fucose wherein the method comprises attaching the fluorophore-conjugated fucose to a glycan to form a labeled glycan.

Aspect F2 is the method of Aspect F1, wherein the fluorophore-conjugated fucose comprises the fluorophore-conjugated fucose of any one of the Exemplary Fluorophore-Conjugated Fucose Aspects (D1-D3).

Aspect F3 is the method of Aspect F1 or F2, wherein the method comprises attaching the fluorophore-conjugated fucose to the glycan using a fucosyltransferase.

Aspect F4 is the method of Aspect F3, wherein the fucosyltransferase comprises FUT1, FUT2, FUT3, FUT4, FUT5, FUT6, FUT7, FUT8, FUT9, FUT10, or FUT11, or a combination thereof.

Aspect F5 is the method of Aspect F3, wherein the fucosyltransferase comprises FUT2, FUT6, FUT7, FUT8, and FUT9, or a combination thereof.

Aspect F6 is the method of any one of Aspects F1 to F5, wherein the method comprises mixing a target protein comprising the glycan with the fluorophore-conjugated fucose, and a fucosyltransferase.

Aspect F7 is the method of any one of Aspects F1 to F6, wherein the method comprises mixing the glycan, the fluorophore-conjugated fucose, and the fucosyltransferase in a buffer.

Aspect F8 is the method of any one of Aspects F1 to F7, wherein the method comprises incubating the glycan, the fluorophore-conjugated fucose, and the fucosyltransferase are incubated together for at least 1 minute and up to 48 hours.

Aspect F9 is the method of any one of Aspects F1 to F8 wherein the glycan, the fluorophore-conjugated fucose, and the fucosyltransferase are incubated together at a temperature of at least 20° C. and up to 50° C.

Aspect F10 is the method of any one of Aspects F1 to F9, wherein the method comprises mixing the glycan with a neuraminidase, a galactosidase, α-2 mannosidase, or MGAT1, or a combination thereof.

Aspect F11 is the method of Aspect F10, wherein the method comprises mixing the glycan with a neuraminidase and a 0-galactosidase or with α-2 mannosidase and MGAT1.

Aspect F12 is the method of Aspect F10 or F11, wherein the method comprises mixing the glycan in the presence of UDP-GlcNAc.

Aspect F13 is the method of any one of Aspects F10 to F12, wherein the method comprises pre-treating the glycan with neuraminidase and galactosidase before mixing the glycan with the fluorophore-conjugated fucose and the fucosyltransferase.

Aspect F14 is the method of any one of Aspects F10 to F12, wherein the method comprises pre-treating the glycan with an α-2 mannosidase and MGAT1 in the presence of UDP-GlcNAc before mixing the glycan with the fluorophore-conjugated fucose and the fucosyltransferase.

Aspect F15 is the method of any one of Aspects F6 to F14, wherein the method comprises attaching the fluorophore-conjugated fucose to the glycan on the target protein to form a labeled target protein comprising the labeled glycan.

Aspect F16 is the method of any one of Aspects F6 to F15, wherein the method further comprises separating components of a mixture comprising the labeled glycan or the labeled target protein comprising the labeled glycan.

Aspect F17 is the method of Aspect F16, wherein separating the components comprises gel electrophoresis of a composition comprising the labeled glycan or a composition comprising the labeled protein.

Aspect F18 is the method of Aspect F16 or F17, wherein the method comprises imaging the labeled glycan or labeled target protein.

Aspect F19 is the method of any one of Aspects F1 to F18, wherein imaging the labeled glycan or the labeled target protein, the fluorophore-conjugated fucose, and/or the fucosyltransferase comprises using silver staining, trichloroethanol (TCE) staining, fluorescent imaging, or a combination thereof.

Aspect F20 is the method of anyone of Aspects F1 to F19, wherein the method comprises cleaving the labeled glycan from the labeled target protein to form a freed labeled glycan.

Aspect F21 is the method of Aspect F20, wherein the method comprises comparing mobility of the freed labeled glycan to mobility of a glycan standard or a glycan ladder, wherein the glycan standard comprises a fluorophore-conjugated glycan and wherein the glycan ladder comprises two or more fluorophore-conjugated glycans.

Aspect F22 is the method of Aspect F21, wherein the glycan ladder comprises a combination of extended glycans.

Aspect F23 is the method of Aspect F22, wherein the extended glycans comprise extended labeled glycans, and wherein the extended labeled glycans comprise fluorophore-conjugated glycans.

Aspect F24 is the method of Aspect F22 or F23, wherein the extended glycans comprise a glycan or glycans extended by one or more glycosyltransferases.

Aspect F25 is the method of F24, wherein the one or more glycosyltransferases comprise MGAT3, MGAT5, B4GalT1, FUT9, or ST3Gal6, or a combination thereof.

Aspect F26 is the method of any one of Aspects F20 to F25, wherein the method comprises gel electrophoresis of a composition comprising the free labeled glycan and imaging the freed labeled glycan.

Aspect F27 is the method of any one of Aspects F6 to F26, wherein the target protein comprises a monoclonal antibody or a viral protein.

Aspect F28 is the method of any one of Aspects F1 to F27, wherein the method further comprises attaching a fluorophore-conjugated sialic acid to a glycan.

### Exemplary Glycan Ladder Aspects

Aspect G1 is a composition comprising a glycan ladder, wherein the glycan ladder comprises at least two labeled glycans.

Aspect G2 is the composition of Aspect G1, wherein the labeled glycans comprise a fluorophore-conjugated glycan.

Aspect G3 is the composition of Aspect G2, wherein the labeled glycans comprise a fluorophore-conjugated sialic acid or a fluorophore-conjugated fucose or both.

Aspect G4 is the composition of Aspect G3, wherein the fluorophore-conjugated sialic acid comprises the fluorophore-conjugated sialic acid of any one of the Exemplary Fluorophore-Conjugated Sialic Acid Aspects (A1-A5).

Aspect G5 is the composition of Aspect G3 or G4, wherein the fluorophore-conjugated fucose comprises the fluorophore-conjugated fucose of any one of the Exemplary Fluorophore-Conjugated Fucose Aspects (D1-D3).

Aspect G6 is the composition of any one of Aspects G1 to G5, wherein the glycan ladder comprises at least three labeled glycans, at least four labeled glycans, at least five labeled glycans, at least six labeled glycans, at least seven labeled glycans, or at least eight labeled glycans.

Aspect G7 is the composition of any one of Aspects G1 to G6, wherein the labeled glycans comprising the glycan ladder comprise a combination of extended glycans.

Aspect G8 is the composition of Aspect G7, wherein the extended glycans comprise extended labeled glycans, and wherein the extended labeled glycans comprise fluorophore-conjugated glycans.

Aspect G9 is the composition of Aspect G7 or G8, wherein the extended glycans comprise a glycan or glycans extended by one or more glycosyltransferases.

Aspect G10 is the composition of G9, wherein the one or more glycosyltransferases comprise MGAT3, MGAT5, B4GalT1, FUT9, or ST3Gal6, or a combination thereof.

Aspect G11 is the composition of any one of Aspects G1 to G10, wherein the glycan ladder comprises G2F2f′, G2f′, N3f′, N2f′, S′1[6]G1f, S′1[6]G1, S2[3]f′, or S2[6]f′.

Aspect G12 is the composition of any one of Aspects G1 to G10, wherein the glycan ladder comprises G2F2f′, G2f′, N3f′, N2f′, S2[3]f′, and S2[6]f′.

Aspect G13 is the composition of Aspect G12, wherein the glycan ladder further comprises S′1[6]G1f and S′1[6]G1.

Aspect G14 is the composition of any one of Aspects E1 to E12, wherein the composition further comprises a buffer compound.

The present invention is illustrated by the following examples. It is to be understood that the particular examples, materials, amounts, and procedures are to be interpreted broadly in accordance with the scope and spirit of the invention as set forth herein.

### EXAMPLES

### Example 1— Direct Fluorescent Glycan Labeling with Recombinant Sialyltransferases

This Example was published as Wu et al. (Wu et al. Glycobiology 29, 750-754 (2019)).

This Example describes using enzymatic incorporation of fluorophore-conjugated sialic acids to achieve the labeling and detection of N- and O-glycans on glycoproteins.

### Material and Methods

CMP-Azido-Sialic acid, recombinant human ST3Gal1, ST3Gal2, ST3Gal4, ST6Gal1, ST6GalNAc1, ST6GalNAc4, MUC1, MUC16, integrin α1β1, α3β1, α5β1, α1β3 and C. perfringens neuraminidase were from R&D Systems (Bio-Techne, Minneapolis, MN). Alexa Fluor® 488 alkyne and Alexa Fluor® 555 alkyne were from Thermo Fisher Scientific (Waltham, MA). Clickable Cy5 or Cy5-alkyne, ascorbic acid, fetal bovine fetuin and asialofetuin were from Sigma-Aldrich (St. Louis, MO).

6×SDS gel loading dye included 9% SDS, 50% Glycerol, and 0.03% Bromophenol blue.

### Preparation of Cytidine Monophosphate Activated Fluorophore-Conjugated Sialic Acid (CMP-f-SA)

Fluorophore-conjugated CMP-f-SA was prepared by incubating equivalent CMP-Azido-Sialic acid (CMP-N₃-SA) and an alkyne-conjugated fluorophore via copper (I)-catalyzed azide-alkyne cycloaddition. In a typical reaction, 5 millimolar (mM) of CMP-N₃-SA was mixed with 5 mM of Cy5-alkyne in the presence of 0.1 mM of Cu²⁺ and 1 mM of ascorbic acid, and the mix was kept at room temperature for 2 hours. Final products were purified on a HiTrap® Q HP column (GE Healthcare, Chicago, IL), eluted with a 0-100% gradient of NaCl elution buffer (300 mM NaCl, 25 mM Tris at pH 7.5) and concentrated to >0.1 mM by a speed-vacuum concentrator.

### Direct Fluorescent Glycan Labeling (DFGL)

For a typical labeling reaction, 1 microgram (μg) to 5 μg target protein was mixed with 0.2 nanomoles (nmol) CMP-f-SA, 0.2 μg of a sialyltransferase in a 30 microliters (μL) buffer of 25 mM Tris pH 7.5, 10 mM MnCl₂, and then incubated at 37° C. for 30 minutes. In the case that the preexisting sialic acid of a glycoprotein needed to be removed, 0.1 microgram (m) of recombinant C. perfringens neuraminidase was also added into the reaction. The neuraminidase showed no activity on fluorophore-conjugated sialic acids and was not removed in most cases. The reaction was then separated on a sodium dodecyl sulfate—polyacrylamide gel electrophoresis (SDS-PAGE) and imaged by a traditional protein imaging station via trichloroethanol (TCE) staining and a fluorescent imager (FluorChem M, ProteinSimple, Bio-Techne, Minneapolis, MN).

### Equipment and Settings

For taking fluorescent image of an SDS gels using FluorChem M (ProteinSimple, Bio-Techne, Minneapolis, MN); multiple fluorescent channel RGB or single fluorescent channel was selected based on the incorporated fluorescent dyes, and the exposure time was set at Auto.

### Results

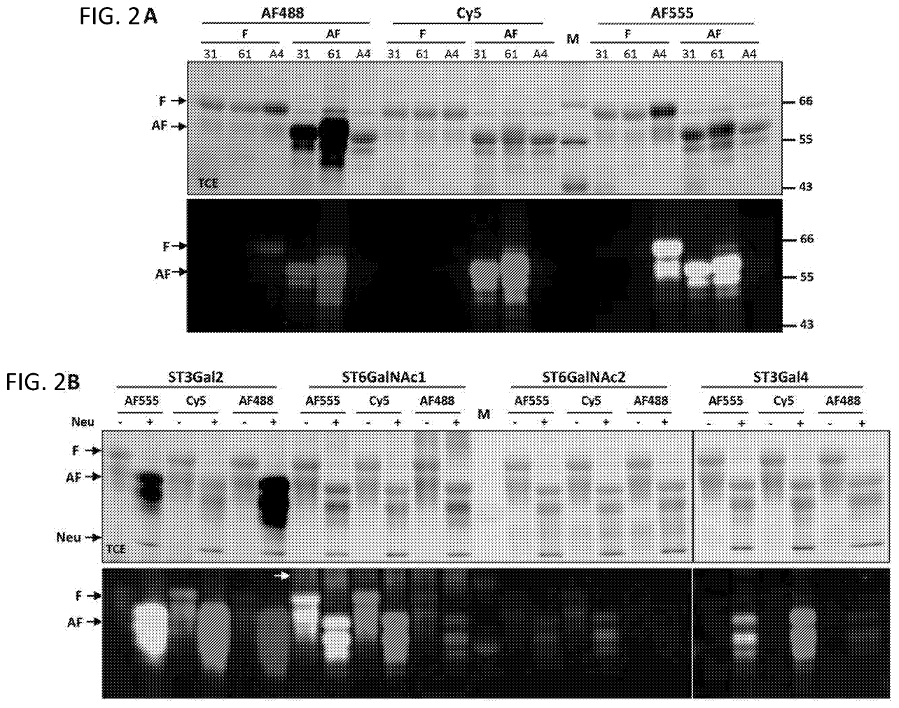

Three cytidine monophosphate activated fluorophore-conjugated sialic acids (CMP-f-SAs) were synthesized by incubating CMP-c5-azido-sialic acid (CMP-N₃-SA) and Alexa Fluor® 555-alkyne, Alexa Fluor® 488-alkyne or Cy5-alkyne via copper (I)-catalyzed azide-alkyne cycloaddition (Rostovtsev et al. Angew Chem Int Ed Engl 41, 2596-2599 (2002)). The synthesized CMP-f-SA was then applied to label the glycans on fetal bovine fetuin and asialofetuin using various sialyltransferases, including Core-1 O-glycan specific ST3Gal1 and ST3Gal2, N-glycan specific ST3Gal4 and ST6Gal1, and, 0-GalNAc specific ST6GalNAc1, ST6GalNAc2 and ST6GalNAc4 (see Table 1). Fetal bovine fetuin is known to contain both N- and O-glycans (Baenziger et al. J Biol Chem 254, 789-795 (1979)) and has historically been used as a model glycoprotein. The labeled reactions were separated by SDS-PAGE and directly imaged with a traditional protein gel imager with trichloroethanol (TCE) staining and a fluorescent gel imager (FIG. 2).

The results indicate that ST3Gal1, ST6Gal1 and ST3Gal4 only labeled asialofetuin; ST3Gal2 primarily labeled asialofetuin, but also weakly labeled fetuin; ST6GalNAc4 only labeled fetuin; ST6GalNAc1 and ST6GalNAc2 labeled both fetuin and asialofetuin (FIG. 2). These results demonstrated that fetal bovine fetuin contains both N- and O-glycans. The strict labeling on asialofetuin by ST6Gal1 and ST3Gal4 also suggests that N-glycans on fetuin are normally fully sialylated. Labeling by ST6GalNAc1, ST6GalNAc2 and ST6GalNAc4 indicated that O-GalNAc residues on fetuin are not fully sialylated. While ST3Gal1 and ST3Gal2 primarily labeled asialofetuin, ST3Gal2 showed some weak labeling on fetuin (FIG. 2, panel B), suggesting that the Gal residues on Core-1 O-glycan are largely sialylated, and that, ST3Gal1 and ST3Gal2 may have slightly different substrate specificities. In addition, the incorporation of Alexa Fluor® fluorophores

TABLE 1
Relative tolerance of the three fluorophores by the sialyltransferases used in this report.

| Sialyltransferases | Acceptor glycan type* | AlexaFluor ™ 555** | AlexaFluor ™ 488** | Cy5* |
|---|---|---|---|---|
| ST3Gal1 | Gal of Core-1 O-glycan | ++ | + | ++ |
| ST3Gal2 | Gal of Core-1 O-glycan | +++ | +++ | +++ |
| ST3Gal4 | Gal of N-glycan | ++ | + | +++ |
| ST6Gal1 | Gal of N-glycan | ++ | +++ | +++ |
| ST6GalNAc1 | O-GalNAc, sialylation on terminal Gal is flexible | ++ | ++ | ++ |
| ST6GalNAc2 | O-GalNAc, sialylation on terminal Gal is flexible | + | − | + |
| ST6GalNAc4 | O-GalNAc, with sialylated terminal Gal | +++ | + | − |

Note:
*Detailed acceptor glycan structures for these enzymes may need further characterization.
**The tolerance of the three fluorophores is based on the corresponding fluorescence intensity of the labeled fetuin or asialofetuin in FIG. 2 and is indicated by + and − signs only. As the fluorescent intensity of each labeled band is also dependent on the abundance of the acceptor glycan for the labeling enzyme on fetuin, comparison of the tolerance of fluorophores by different labeling enzymes is not valid.

greatly increased band intensities in TCE images, which is likely due to the presence of multiple benzene rings in these dyes.

Although some of the sialyltransferases tolerated the three fluorophores equally well, some enzymes showed a preference. For example, ST6GalNAc4 showed strong preference for Alexa Fluor® 555 over Cy5, while ST6GalNAc2 showed preference for Cy5 over Alexa Fluor® 488 (FIG. 2). The tolerances of the three fluorophores by these sialyltransferases are summarized in Table 1.

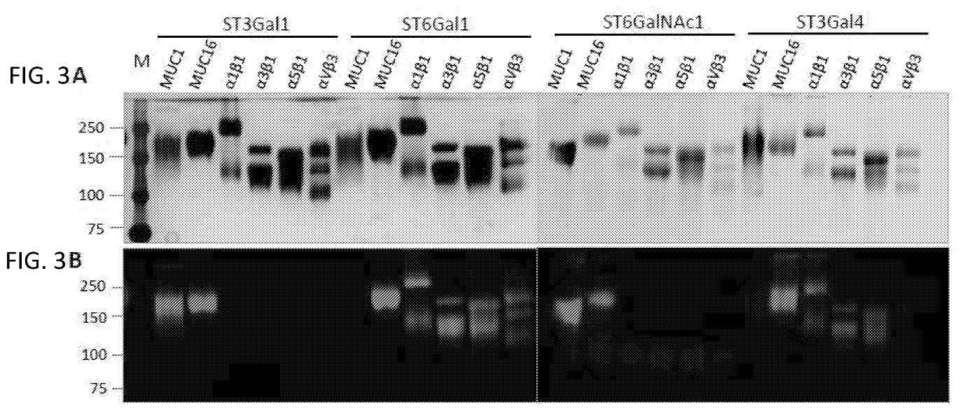

To further test the specificity of labeling by these sialyltransferases, representative mucins and integrins were labeled with Cy5 using 0-glycan specific ST3Gal1 and ST6GalNAc1, and N-glycan specific ST6Gal1 and ST3Gal4. Mucins are known to be abundant in O-glycans (Tran et al. J Biol Chem 288, 6921-6929 (2013)) and integrins are known to be abundant in N-glycans (Gu et al. Glycoconj J 21, 9-15 (2004)). MUC16 in particular contains both N- and O-glycans (Taniguchi et al. J Biol Chem 292, 11079-11090 (2017)). Indeed, it was found that MUC1 was strictly labeled by ST3Gal1 and ST6GalNAc1, all integrins were strictly labeled by ST6Gal1 and ST3Gal4, and MUC16 was labeled by all four enzymes (FIG. 3).

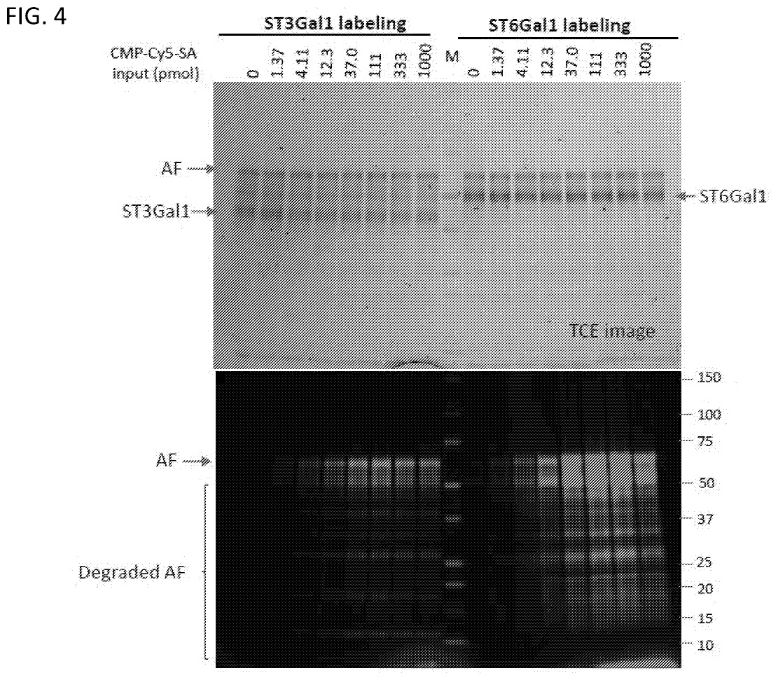

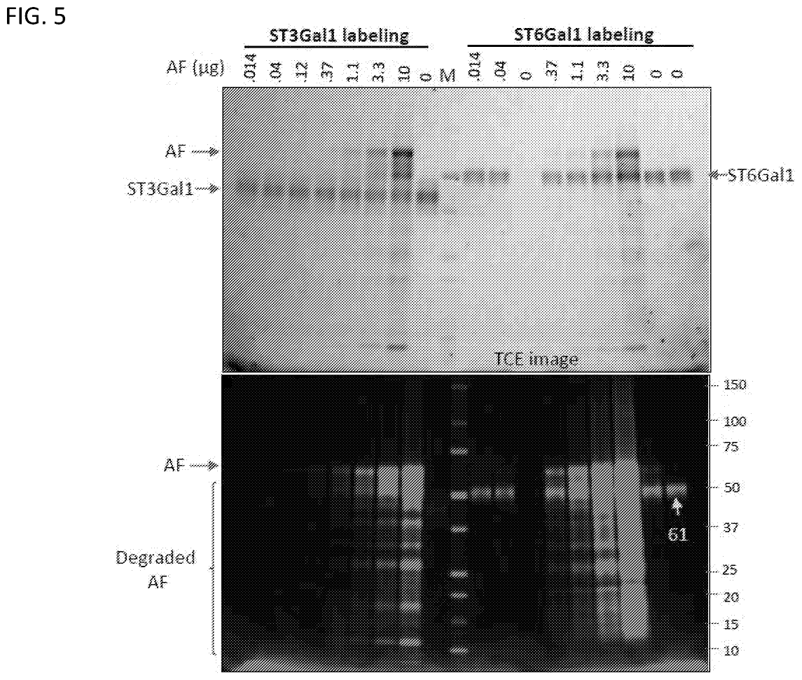

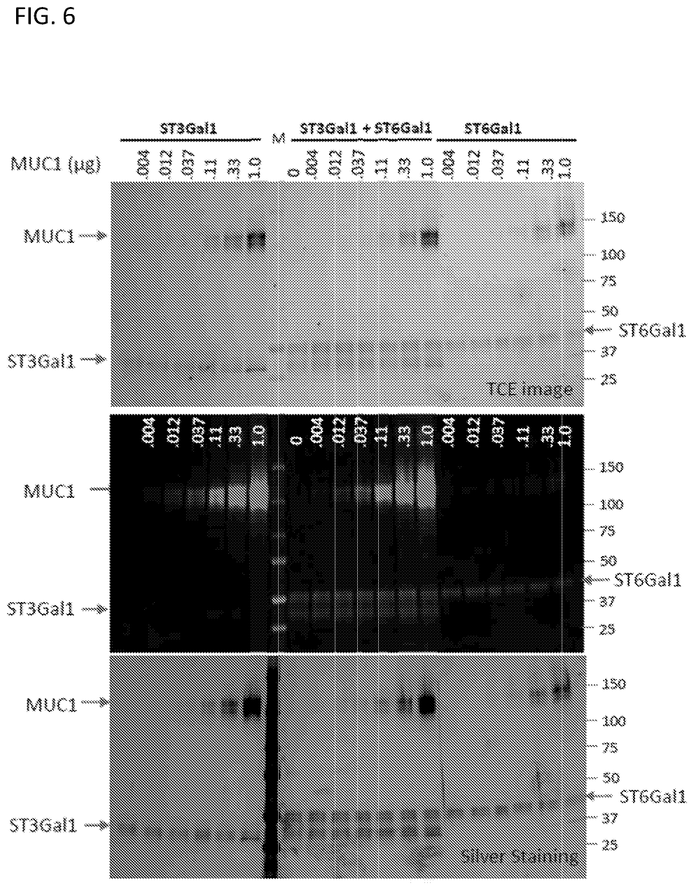

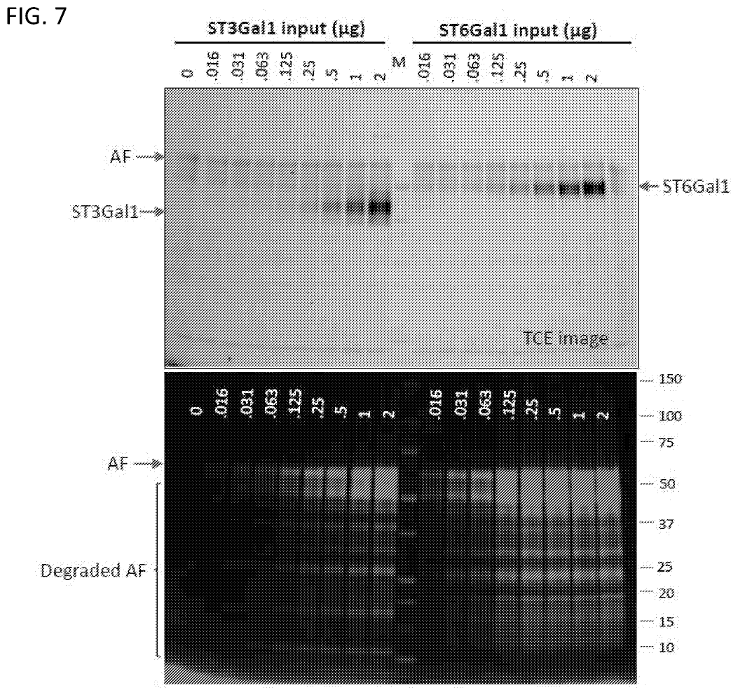

Furthermore, the sensitivity of the labeling regarding both the donor and acceptor substrates and the enzymes themselves were tested. It was found that the lower limits for detection was achieved at micromolar level of CMP-Cy5-SA (see FIG. 4, which shows that 1.211M of CMP-Cy5-SA is needed for maximal labeling of asialofetuin (AF) by both ST3Gal1 and ST6Gal1), sub-microgram levels of fetuin (see FIG. 5, which shows that the lower detection limit for AF labeled by ST3Gal1 and ST6Gal1 could be much lower than 0.37 μg), nanogram levels of MUC1 (see FIG. 6, which shows that the lower limit of detection for MUC1 labeled by ST3Gal1 is around 0.012 μg), and sub-microgram levels of labeling enzymes (see FIG. 7, which shows that 0.5 μg of ST3Gal1 is needed for maximal labeling of AF and 0.25 μg of ST6Gal1 is needed for maximal labeling of AF).

Resialylation with fluorophore-conjugated sialic acids did not obviously reduce the mobility of the target protein in SDS-PAGE (FIG. 2, FIG. 4, and FIG. 7). This phenomenon might be explained by the presence of multiple negative charges carried by these fluorophores. The net increase of a protein's negative charge may result in increased mobility in SDS-PAGE, counteracting the mobility reduction due to the increased molecular mass.

### Example 2— Detecting Substrate Glycans of Fucosyltransferases on Glycoproteins with Fluorophore-Conjugated Fucose

A modified version of this Example was published as Wu et al. (Wu et al. Glycobiology, cwaa030 (2020)).

This Example describes using enzymatic incorporation of fluorophore-conjugated sialic acids to achieve the labeling and detection of N- and O-glycans on glycoproteins to determine the differential distribution of N- and O-glycans and variable expression of sialyl-T antigen on HeLa cells.

### Materials and Methods

Recombinant fucosyltransferases FUT2, FUT6, FUT8, FUT9, MGAT1, B4GalT1, ST6Gal1, H1N1 viral neuraminidase, C. perfringens neuraminidase and GDP-Azido-Fucose were from Bio-Techne (Minneapolis, MN). Cantuzumab, an anti-Muc1 therapeutic antibody, was from Creative Biolabs (Shirley, NY). NIST monoclonal antibody reference material 8671 was from the National Institute of Standards and Technology (Gaithersburg, MD). Alkyne-Alexa Fluor® 488 and alkyne-Alexa Fluor® 555 were from Thermo Fisher Scientific (Waltham, MA). Cy5-alkyne, RNase B, fetal bovine fetuin and asialofetuin and all other chemical reagents were from Sigma-Aldrich (St. Louis, MO).

### Preparation of Guanosine 5′-diphosphate activated fluorophore-conjugated fucose (GDP-f-Fuc)

Activated fluorophore-conjugated fucoses (GDP-f-Fucs) were prepared by incubating equivalent GDP-Azido-Fucose (GDP-N₃-Fuc) and an alkyne-conjugated fluorophore via copper (I)-catalyzed azide-alkyne cycloaddition. As an example, 5 mM of GDP-N₃-Fuc was mixed with 5 mM of Cy5-alkyne in the presence of 0.1 mM of Cu′ and 1 mM of ascorbic acid. The reaction was maintained at room temperature for 2 hours. The synthesized GDP-f-Fuc was then purified on a HiTrap® Q HP (GE Healthcare, Chicago, IL) column and eluted with a 0-100% gradient of NaCl elution buffer (300 mM NaCl, 25 mM Tris at pH 7.5). The GDP-f-Fuc was collected based on color exhibition and UV absorption; GDP-f-Fuc was vivid in color and had UV absorption at 260 nm. Guanosine 5′-diphosphate activated Alexa Fluor® 555-conjugated fucose (GDP-AF555-Fuc, guanosine 5′-diphosphate activated Alexa Fluor® 488-conjugated fucose (GDP-AF488-Fuc), guanosine 5′-diphosphate activated Cy5-conjugated fucose (GDP-Cy5-Fuc) were prepared and purified accordingly and concentrated to >0.1 mM by a speed-vacuum concentrator.

### Fluorescent Labeling of Glycoproteins Using Fucosyltransferases

For a typical labeling reaction, 1 μg to 5 μg target protein was mixed with 0.2 nmol GDP-f-Fuc and 0.2 μg of a fucosyltransferase in 30 μL buffer of 25 mM Tris pH 7.5, 10 mM MnCl₂. The mixture was incubated at 37° C. for 30 minutes. The reaction was then separated by sodium dodecyl sulfate—polyacrylamide gel electrophoresis (SDS-PAGE) and the gel was directly imaged using a fluorescent imager FluorChem M ProteinSimple, Bio-Techne, Minneapolis, MN), followed by imaging with traditional protein imaging methods such as silver staining or trichloroethanol (TCE) staining.

### Gly-Q™ Analysis

All samples for Gly-Q™ analysis were prepared analyzed according to the manufacture's protocol (Prozyme, Inc., Agilent Technologies, Santa Clara, CA).

### Results

### Detection of Substrate Glycans of α-2 and α-3 Fucosyltransferases on Fetal Bovine Fetuin

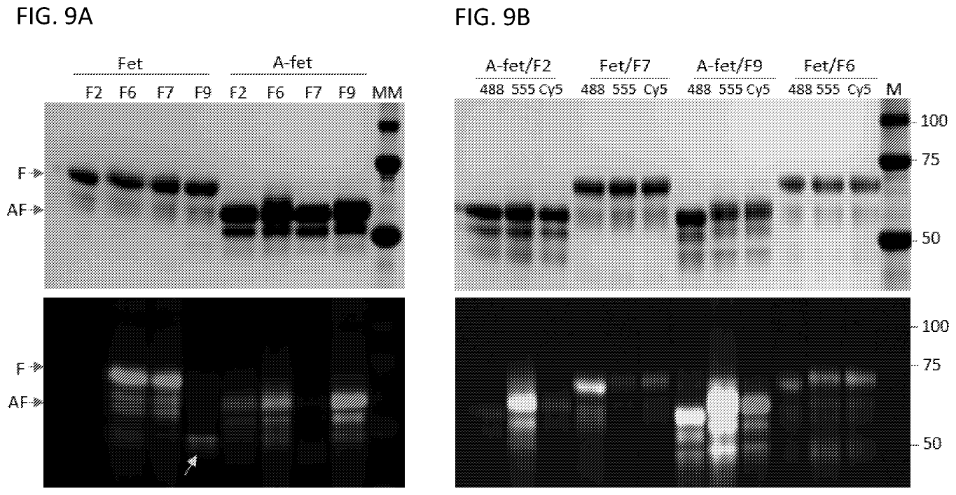

To test for detection of the substrate glycans of α-2 and α-3 fucosyltransferases, GDP-Cy5-Fuc was prepared and tested as a donor substrate for FUT2, FUT6, FUT7, and FUT9 on fetal bovine fetuin and asialofetuin (FIG. 9A). The labeled samples were then separated on SDS-PAGE, followed by traditional protein gel imaging and fluorescent imaging. By comparing the images, it was found that these enzymes may recognize Cy5-conjugated fucose (Cy5-Fuc) and labeled their substrate glycans. Specifically, FUT2 and FUT9 labeled asialofetuin; FUT7 labeled fetuin; FUT6 labeled both fetuin and asialofetuin. These results are consistent to the previously reported specificities of these enzymes (Mondal et al. J Biol Chem 293, 7300-7314 (2018), Sasaki et al. J Biol Chem 269, 14730-14737 (1994), Brito et al. Biochimie 90, 1279-1290 (2008), Weston et al. J Biol Chem 267, 24575-24584 (1992)).

FUT2, FUT6, FUT7, and FUT9 were also evaluated for their tolerance towards Cy5-, AlexaFluor® 488-, and AlexaFluor® 555-conjugated fucoses (FIG. 9B). The four enzymes tolerated the three fluorophores to different levels. For example, FUT2 preferred AlexaFluor® 555, FUT7 preferred AlexaFluor® 488, FUT9 preferred AlexaFluor® 555, and FUT6 showed no obvious preference among the three fluorophore-conjugated fucoses (see also Table 2).

TABLE 2
Fucosyltransferase used in this study and their tolerance* for Cy5,
AlexaFluor ® 488, and AlexaFluor ® 555

| | | Alexa | Alexa | |
|---|---|---|---|---|
| Fucosyl- | | Fluor ® | Fluor ® | |
| transferases | Acceptor glycan type | 555 | 488 | Cy5 |
| FUT2 | Terminal Gal of | +++ | + | + |
| | lactosamine | | | |
| FUT6 | GlcNAc of lactosamine, | ++ | ++ | ++ |
| | sialylation on terminal | | | |
| | Gal is flexible | | | |
| FUT7 | GlcNAc of | + | ++ | + |
| | sialylactosamine | | | |
| FUT8 | Core GlcNAc of | ++ | ++ | +++ |
| | N-glycans | | | |
| FUT9 | GlcNAc of lactosamine | +++ | +++ | +++ |

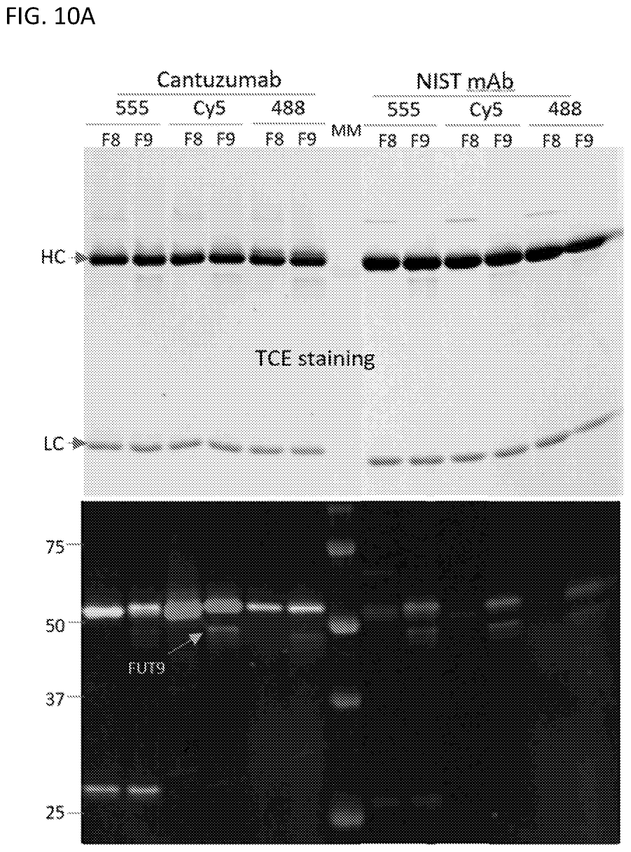

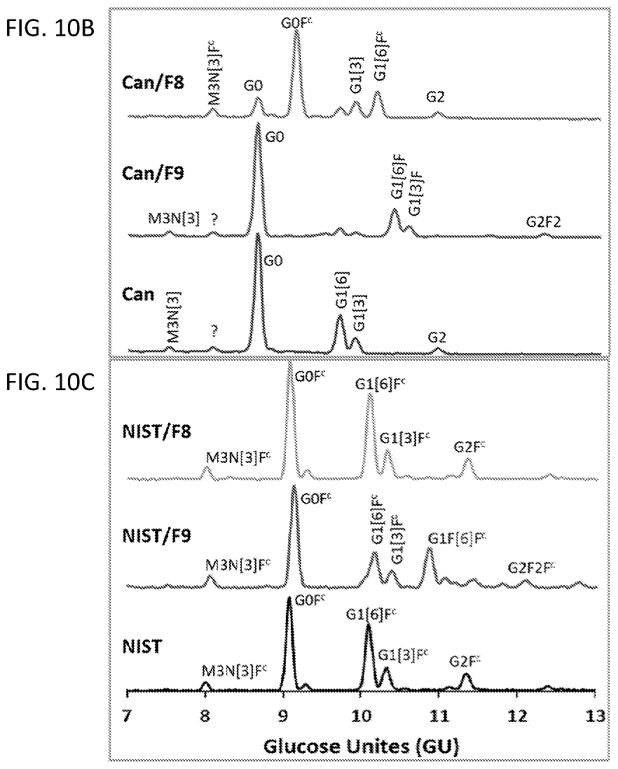

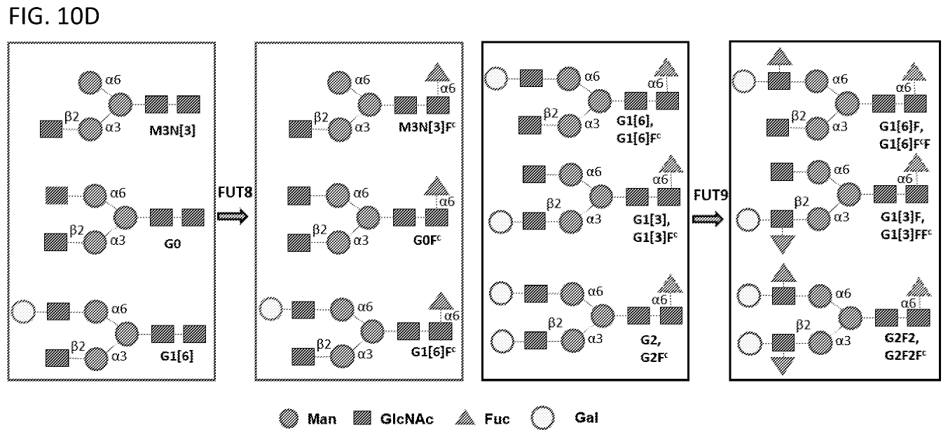

Note:
*The tolerance of fluorophores by fucosyltransferase is based on the corresponding fluorescent intensity of the labeled fetuin or asialofetuin in FIG. 9, or labeled Cantuzumab in FIG. 10, and, is indicated by number of + signs. As the fluorescent intensity of each labeled band is also dependent on the abundance of the glycan acceptor on a target protein, comparison of the tolerance of different fluorophores should be limited to those under a same labeling enzyme.

### Probing the Status of Core-6 Fucosylation on Therapeutic Antibodies by FUT8

Although FUT8 was known to tolerate azido-fucose (Wu et al. Biochem Biophys Res Commun 473, 524-529 (2016)), it was unknown whether FUT8 could tolerate a fluorophore-conjugated fucose. To test whether FUT8 could tolerate the fluorophore-conjugated fucose and detect its substrate glycans, two substrates were used: Cantuzumab was prepared from a FUT8 knockout cell line, and a reference monoclonal antibody from the National Institute of Standards and Technology (NIST mAb, material 8671, Gaithersburg, MD). The NIST mAb is a humanized IgG1κ monoclonal antibody (Kashi et al. MAbs 10, 922-933 (2018)). IgG antibodies are known to contain an N-glycan site on their heavy chains (Reusch et al. Glycobiology 25, 1325-1334 (2015)).

FIG. 10A shows that significant amounts of Alexa-Fluor® 555, Alexa-Fluor® 488 and Cy5 conjugated fucoses were introduced into Cantuzumab but not to NIST mAb by FUT8. For comparison, the samples were also probed by FUT9, which showed consistent incorporation of the three dyes to into both antibodies (FIG. 10A).

To determine if the labeling was specific to respective enzyme substrate glycans, in a parallel experiment, glycans of in vitro fucosylated Cantuzumab and NIST mAb were analyzed on a Gly-Q™ Glycan Analysis System (Prozyme, Inc., Agilent Technologies, Santa Clara, CA) (FIG. 10B). While FUT9 converted G1[6], G1[3] and G2 of Cantuzumab to G1[6]F, G1[3]F, and G2F2, respectively, FUT8 converted M3N[3], G0, and G1[6] of the antibody to M3N[3]F^{c}, G0F^{c} and G1[6]F^{c}, respectively, demonstrating the strict specificities of these two fucosyltransferases. Consistent to the labeling results of FIG. 10A, glycan analysis of NIST mAb suggested that G1[6]F^{c} and G2F^{c} were modified by FUT9 but no detectable substrate glycans for FUT8 were found (FIG. 10C). In fact, all peaks in the electropherogram of NIST mAb were found to be core-6 fucosylated. Schematics of the structures of glycans analyzed are shown in FIG. 10D.

### Detecting High Mannose Glycans by FUT8

High mannose N-glycans may affect serum clearance of therapeutic antibodies (Goetze et al. Glycobiology 21, 949-959 (2011)) and are frequently targeted in broad neutralizing antibody responses during human immunodeficiency viral infection (Lavine et al. J Virol 86, 2153-2164 (2012)); therefore, detection of high-mannose glycan would be particularly valuable. Thus, a strategy to probe high mannose glycans using FUT8 that demonstrates the substrate specificity of FUT8 was developed.

Bovine ribonuclease B (RNase B) is known to contain high-mannose glycans (Prien et al. J Am Soc Mass Spectrom 20, 539-556 (2009)). To test whether high-mannose glycans may be detected on a glycoprotein, RNase B labeling by FUT8 and GDP-Cy5-Fuc was evaluated. No labeled product was observed when the sample was not pretreated by MGAT1, FUT8, B4GalT1 and ST6Gal1 in the presence of their native donor substrates. (FIG. 11A, lane a.) In contrast, a sample of RNase B that was treated with MGAT1 to introduce the α1-3-arm GlcNAc residue before labeling by FUT8 (see FIG. 8, panel D) resulted in the addition of α1-3-arm GlcNAc residue by MGAT1 and in strong labeling by FUT8. Further galactosylation and sialylation significantly reduced the labeling (left side of FIG. 11, panel A). In addition, pretreatment of RNase B with unmodified fucose by FUT8 abolished the labeling completely, suggesting that the modification of fucose by Cy5 did not affect the substrate recognition by FUT8. As a positive control, an RNase B sample pretreated with MGAT1 and B4GalT1 was labeled with Cy5 conjugated sialic acid by ST6Gal1, which resulted in similar intensity of labeling by FUT8.

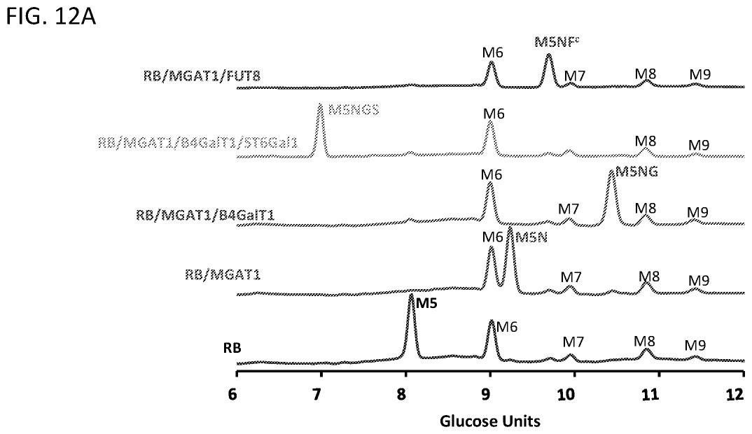

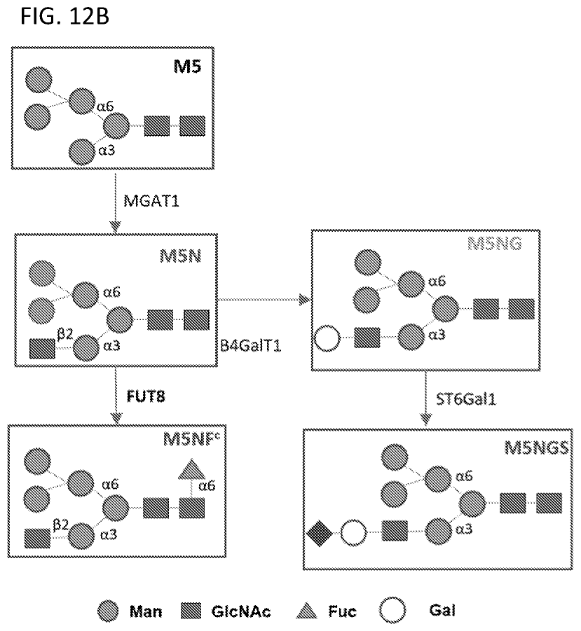

To confirm that the glycans labeled by FUT8 and ST6Gal1 were high-mannose glycans, sequentially modified RNase B samples were analyzed with Gly-Q™ Glycan Analysis System. The results indicated that only Man5 (M5) was modified by FUT8 via MGAT1 (FIG. 11, panel B) and ST6Gal1 via MGAT1 and B4GalT1 (FIG. 12). Since other high-mannose glycans including Man6, Man7, Man8, and Man9 may be converted to Man5 by α1,2 specific mannosidase treatment (Avezov et al. Mot Blot Cell 19, 216-225 (2008)), in theory, all these glycans may be detected by FUT8 as well.

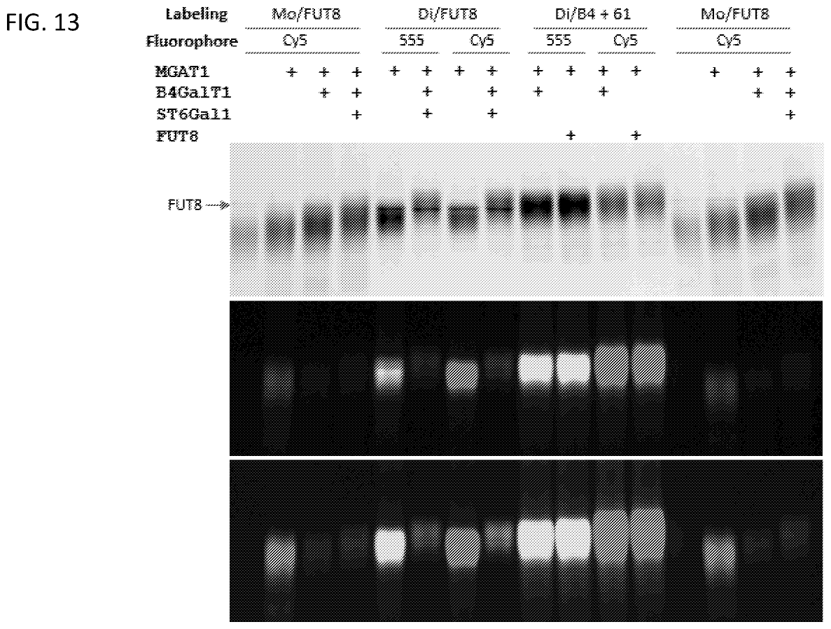

To test whether Man3 glycan may be labeled, monomeric Sf21 cell expressed recombinant 1918 H1N₁ influenza neuraminidase (H1N1 Neu) that is known to contain both Man3 (M3) and core-6 fucosylated Man3 (M3F^{c}) (Wu et al. Biochem Biophys Res Commun 473, 524-529 (2016)) was labeled by FUT8. Again, the sample was labeled significantly by FUT8 only after pretreatment with MGAT1 and the labeling was inhibited or abolished by additional pretreatment by B4GalT1 and ST6Gal1 (right side of FIG. 11, panel A), again confirming that an unmodified GlcNAc introduced by MGAT1 on the α1,3-arm of high-mannose N-glycan is critical for FUT8 recognition. Meanwhile, since the difference of Man5 and Man3 is on their α1,6-arms, these results also proved that the α1,6-arms are flexible for FUT8 recognition. In contrast to labeling on RNase B, the signal of FUT8 labeled product was only a fraction of that of ST6Gal1 labeled product. To understand this difference and confirm that Man3 was indeed modified, sequentially modified Neu samples were subject to Gly-Q™ analysis. It was found that the precursor substrate glycan for FUT8 (M3) on H1N1 Neu was only about 3% of that of the precursor substrate glycans for ST6Gal1 (both M3 and M3F^{c}) (FIG. 11, panel C), therefore explaining the difference on the signals labeled by FUT8 and ST6Gal1 in FIG. 11, panel A. Similar results were obtained when the experiment was repeated on both monomeric and dimeric recombinant 1918 H1N₁ influenza neuraminidase prepared in different batches (FIG. 13).

These experiments demonstrated that an unmodified α1-3 arm GlcNAc residue introduced by MGAT1 is important for FUT8 recognition; extension of the 1-3 arm GlcNAc residue by B4Gal1 and ST6Ga1 significantly inhibits FUT8 recognition; and while extension of the α1-3 arm GlcNAc residue by B4GalT1 and ST6Gal1 inhibits core-6 fucosylation, core-6 fucosylation has no obvious effect on ST6Gal1 substrate recognition.

Interplay between Sialylation and Fucosylation Revealed by Simultaneous Labeling of Fucose and Sialic Acid

Since fucosylation and sialylation involve different donor substrates, it may be possible to label a common substrate glycan with fucosyltransferases and sialyltransferases simultaneously, and thereby, to study the interplay between these two families of enzymes.

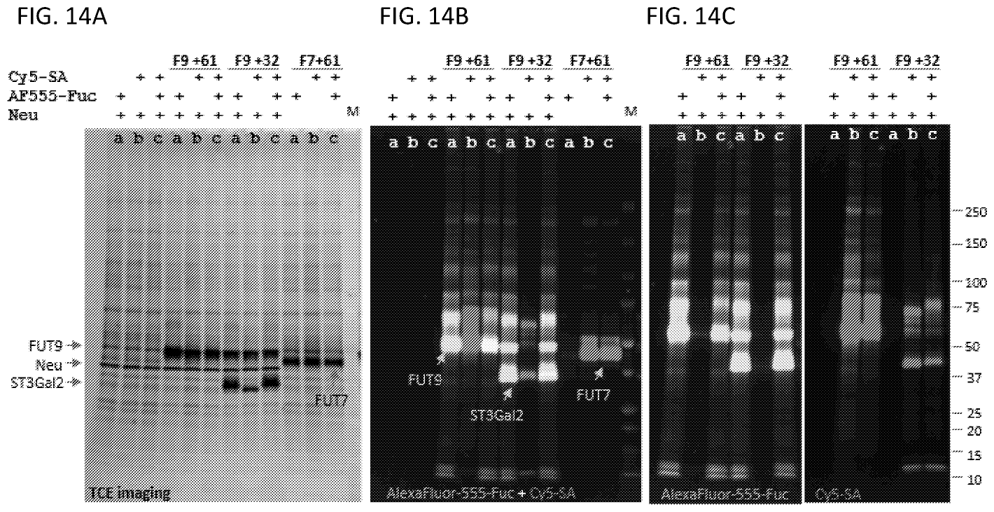

To test this hypothesis, cytoplasmic extracts of HEK293 cells were labeled simultaneously by a sialyltransferase (ST6Gal1 or ST3Gal2) and a fucosyltransferase (FUT7 or FUT9) (FIG. 14). ST6Gal1 is active on terminal Galβ1-4GalNAc disaccharide on N-glycans (Wu et al. Glycobiology 21, 727-733 (2011), Wu et al. Glycobiology 26, 329-334 (2016)). ST3Gal2 is active on terminal Galβ1-3GalNAc disaccharide found on O-glycans (Kitagawa et al. J Biol Chem 269, 1394-1401 (1994), Wu et al. Glycobiology 26, 329-334 (2016)). To generate substrate glycans for FUT9, samples were pretreated with recombinant C. perfringens neuraminidase to remove terminal sialic acids. The four enzymes exhibited distinctive labeling patterns, especially at bands around 10 kDa (likely to be labeled glycopeptides), demonstrating these enzymes recognize overlapping but distinct glycan substrates (FIG. 14, panel B). More specifically, FUT9 and ST6Gal1 exhibited largely overlapping but not identical labeling pattern. When the extract was labeled simultaneously by FUT9 and ST6Gal1, the signal generated by either of the two enzymes was reduced compared to the signals generated by them individually (FIG. 14, panel C), suggesting that the two enzymes have mutually exclusive relationship on their substrate recognition, that is, sialylation by ST6Gal1 prevents fucosylation by FUT9 and vice versa. In contrast, ST3Gal2 showed no obvious interference to FUT9 labeling. ST6Gal1 and FUT7 exhibited very weak labeling on samples without neuraminidase treatment, suggesting that the proteins in the extracts were largely α2-6 sialylated and did not contain the substrate glycans for FUT7.

### Example 3— Detecting Substrate Glycans of Fucosyltransferases with Fluorophore-Conjugated Fucose and Methods for Glycan Electrophoresis

A modified version of this Example was published as Wu et al. (Wu et al. Glycobiology, cwaa030 (2020)).

This Example describes the detection of the substrate glycans of fucosyltransferases on glycoproteins as well as in their free forms via enzymatic incorporation of fluorophore-conjugated fucose using FUT2, FUT6, FUT7, and FUT8 and FUT9. Specifically, the detection of the substrate glycans of these enzymes on fetal bovine fetuin, recombinant H1N1 viral neuraminidase and therapeutic antibodies is described. The detected glycans include complex and high-mannose N-glycans. Establishing a series of precursors for the synthesis of Lewis X and sialyl Lewis X structures, not only provides convenient electrophoresis methods for studying glycosylation but also demonstrates the substrate specificities and some kinetic features of these enzymes. These results support the notion that fucosyltransferases are key targets for regulating the synthesis of Lewis X and sialyl Lewis X structures.

As further described herein, the methods were demonstrated on several well characterized glycoproteins, including fetal bovine fetuin that contains complex N-glycans and O-glycans (Ma et al. Glycobiology 16, 158R-184R (2006)), ribonuclease B that contains high-mannose N-glycans (Prien et al. J Am Soc Mass Spectrom 20, 539-556 (2009)), insect cell expressed recombinant H1N1 neuraminidase that contains Man3 type high-mannose N-glycan (Wu et al. Biochem Biophys Res Commun 473, 524-529 (2016)), Cantuzumab (Rodon et al. Cancer Chemother Pharmacol 62, 911-919 (2008)) and the reference monoclonal antibody from National Institute of Standards and Technology (NIST mAb 8671) (Kashi et al. MAbs 10, 922-933 (2018)) that contains complex N-glycans. By establishing a series of precursor glycans through enzymatic conversion, these results reveal multiple intermediate products during the synthesis of Le^{x} and sLe^{x}. The results indicate that fucosylation is a much fast process than sialylation, suggesting that fucosylation is the step where the synthesis of Le^{x} and sLe^{x} is controlled.

### Materials and Methods

Recombinant fucosyltransferases and activated fluorophore-conjugated fucoses (GDP-f-Fucs) were prepared as described in Example 2.

### Fluorescent Labeling of Glycoproteins and Glycans and Separation of Labeled Sample on SDS-PAGE

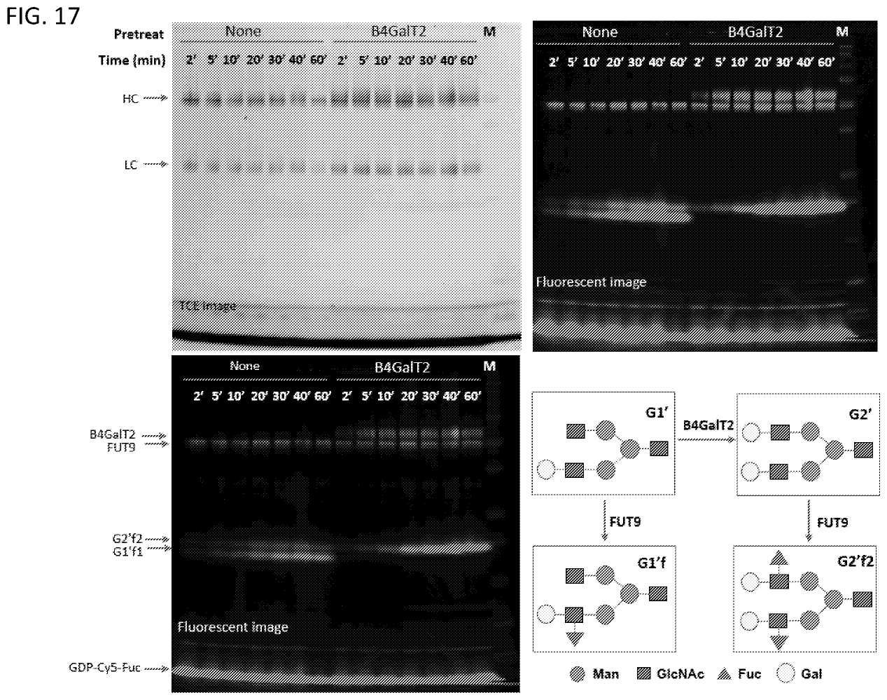

For a typical labeling reaction, 1 μg to 5 μg target protein was mixed with 0.2 nmol fluorophore-conjugated GDP-fucose and 0.2 μg of a fucosyltransferase in 30μL 25 mM Tris pH 7.5, 10 mM MnCl₂. The mixture was incubated at 37° C. for 30 minutes. Longer incubation may increase labeling but not significantly (FIG. 17). To release labeled glycans from a glycoprotein, the sample was first denatured by heating at 95° C. for two minutes in the presence of 0.5% SDS and 80 mM β-mercaptoethanol and then renatured with 1% Triton X-100, and finally treated with PNGase F at 10:1 mass ratio at 37° C. for 20 minutes. To release glycans from antibody, a sample was directly treated with Endo S at 10:1 mass ratio at 37° C. for 20 minutes. All samples were separated by sodium dodecyl sulfate—polyacrylamide gel electrophoresis (SDS-PAGE) at volts/cm. For separating labeled glycoproteins and antibodies, 4-20% gradient SDS gel was used. For separating labeled free glycans, 15% or 17% gel was used. After separation, all gels were imaged using a FluorChem M imager (ProteinSimple, Bio-Techne, Minneapolis, MN). For glycoproteins samples, the gel was also imaged with traditional methods such as silver staining or trichloroethanol (TCE) staining.

### GlyQ Analysis

All samples for GlyQ analysis were prepared and analyzed according to the manufacture's protocol in Agilent Gly-Q™ Glycan Analysis System (formerly ProZyme).

### Results

### Detection of Substrate Glycans of α-2 and α-3 Fucosyltransferases on Fetal Bovine Fetuin

As described in Example 2, FUT2, FUT6, FUT7, and FUT9 tolerated Cy5-, AlexaFluor® 488-, and AlexaFluor® 555-conjugated fucoses to different levels.

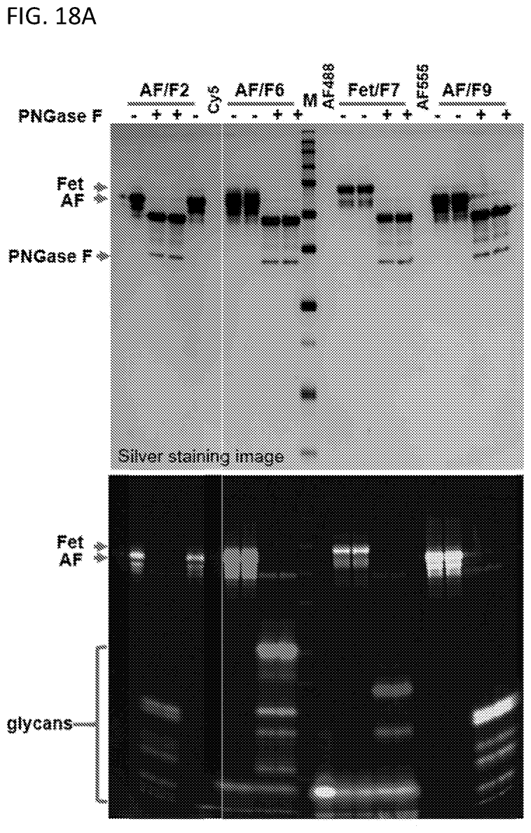

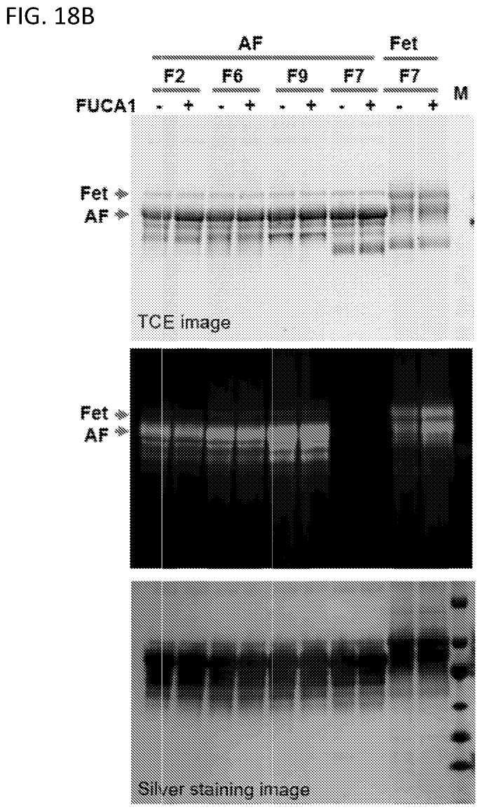

To further understand the nature of the glycans labeled by these enzymes, samples were either treated with PNGase F, an amidase that removes entire N-glycans from glycoproteins (Tarentino et al. Methods Enzymol 230, 44-57 (1994)), or FUCA1, a lysosomal enzyme that hydrolyze α-fucose residues from glycans (Fukushima et al. Proc Natl Acad Sci USA 82, 1262-1265 (1985)). When the FUT2-, FUT6-, FUT7-, and FUT9-labeled samples were treated with PNGase F, all incorporated fluorescent signals were released (FIG. 18A), suggesting that the substrate glycans for those enzymes on fetuin are exclusively carried by N-glycans. This experiment also demonstrated that free N-glycans can be separated well by gel electrophoresis. Among FUCA1 treated samples, two-fold increase on labeling was observed on FUT7 labeled fetuin after the treatment (FIG. 18B), suggesting the preexistence of sialyl Lewis X on native fetuin sample.

### Probing Fucosylation on Therapeutic Antibodies by FUT8 and FUT9

The substrate glycans for FUT8 and FUT9 on Cantuzumab and the NIST mAb were first evaluated as described in Example 2 (see FIG. 10).

To further identify the substrate glycans for FUT8 and FUT9 on Cantuzumab and the NIST mAb, a glycan ladder was established via enzymatic conversion of FUT8-labeled G0 glycan (FIG. 19A) and then compared the glycans released by Endo S and PNGase F from the two antibodies to the ladder (FIG. 19B). Endo S is an endoglycosidase specific for the glycans on IgG and its cleavage on IgG leaves the innermost GlcNAc residue of a target N-glycan attached to the protein backbone (Collin et al. EMBO J 20, 3046-3055 (2001)). One major PNGase F released glycan from Cantuzumab matched G0 through FUT8 labeling and the labeling was sensitive to B4Galt1 pretreatment, suggesting that the glycan is G0. FUT8 showed no labeling on Endo S released glycans, as these glycans lacked glycosylation sites for FUT8. FUT9 labeling resulted one major lower band and one minor upper band on both Endo S and PNGase F released glycans from both Cantuzumab and the NIST mAb, with the intensities of the two bands corresponding well with the peak intensities of G1 and G2 species in the GlyQ data of FIG. 10. Moreover, the lower bands in FIG. 19B were shifted to the upper bands by B4GalT1 treatment, suggesting that the two bands are corresponding to G1 and G2, respectively.

These results not only demonstrated that the substrate glycans on antibodies can be labeled and detected but also demonstrated that glycans that differ by one sugar residue such as G1′f, G0f, and G1f in FIG. 19B and even glycan isomers such as A2[6] and A2[3] in FIG. 19A can be separated in SDS-PAGE.

### Detecting High Mannose Glycans by FUT8

As described in Example 2, high-mannose glycans may be detected on a glycoprotein.

To further test whether high-mannose glycans can be detected on a glycoprotein, a sample of RNase B was first treated with α1,3-mannosyl-glycoprotein 2-β-N-acetylglucosaminyltransferase (MGAT1) to introduce the α3 arm GlcNAc residue before labeling by FUT8 (FIG. 11A). The addition of α3 arm GlcNAc residue by MGAT1 resulted in strong labeling by FUT8, and further galactosylation and sialylation significantly reduced the labeling (left side of FIG. 11A), which is consistent to the result in FIG. 19B and the observation that G1[3] was not modified by FUT8 in FIG. 10. In addition, pretreatment of RNase B by FUT8 with unmodified fucose abolished the labeling completely, suggesting that the conjugation of Cy5 to fucose did not affect the substrate recognition by FUT8. As a positive control, an RNase B sample pretreated with MGAT1 and β-1,4-galactosyltransferase 1 (B4GalT1) was labeled with Cy5-conjugated sialic acid by ST6Gal1, which resulted in similar intensity of labeling (comparing lane b and fin FIG. 11A).

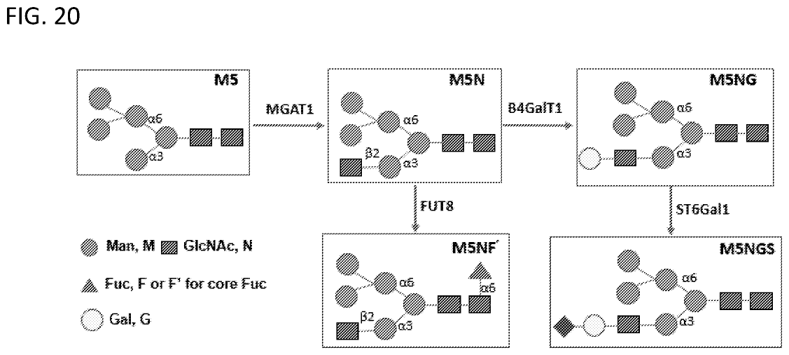

To identity the glycans labeled by FUT8 and ST6Gal1, sequentially modified RNase B samples were analyzed with Gly-Q™ Glycan Analysis System. The results indicated that only Man5 (M5) led to eventual modification by FUT8 (FIG. 11B) and ST6Gal1 (FIG. 20; see also FIG. 12A). Since other high-mannose glycans including Man6, Man7, Man8, and Man9 can be converted to Man5 by α1,2 specific mannosidase (Avezov et al. Mol Biol Cell 19, 216-225 (2008)), in theory, all these glycans can be detected by FUT8 as well.

To test whether Man3 glycan can be labeled, monomeric Sf21 cell expressed recombinant 1918 H1N₁ influenza neuraminidase (Neu) that is known to contain both Man3 (M3) and core-6 fucosylated Man3 (M3F) (Wu et al. Biochem Biophys Res Commun 473, 524-529 (2016)) was tested by FUT8. Again, the sample was labeled significantly by FUT8 only after pretreatment with MGAT1 and the labeling was inhibited or abolished by additional pretreatment by B4GalT1 and ST6Gal1 (right side of FIG. 11A). Meanwhile, since the difference between Man5 and Man3 is on their α6 arms, these results also proved that the α6 arms are flexible for FUT8 recognition. In contrast to labeling on RNase B, the signal of FUT8 labeled Neu was only a fraction of that of ST6Gal1 labeled Neu (comparing lane i and m in FIG. 11A). To understand this difference and confirm that Man3 was indeed modified, sequentially modified Neu samples were subject to GlyQ analysis. It was found that the precursor substrate glycan for FUT8 (M3) on Neu was only about 3% of that of the precursor substrate glycans for ST6Gal1 (both M3 and M3F) (FIG. 11C), thereby explaining the difference on Neu samples labeled by FUT8 and ST6Gal1 in FIG. 11A. Similar results were obtained when the experiment was repeated on both monomeric and dimeric recombinant 1918 H1N₁ influenza neuraminidase prepared in different batches (FIG. 13).

Examination of the Enzymatic Synthesis of Le^{x} and sLe^{x} Epitopes Using Glycan Gel Electrophoresis

As a further demonstration of glycan gel electrophoresis, the enzymatic synthesis of Le^{x} and sLe^{x} based on the antibody glycan G0 were examined. For Le^{x} synthesis, G0 was first converted to G2 by B4GalT1 and then converted to G2F2 (carrier of Le^{x}) by FUT6 or FUT9. For sLe^{x} synthesis, G0 was first converted to G2 by B4GalT1, then converted to A2[3] by ST3Gal4, and finally converted to A2[3]F2 (carrier of sLe^{x}) by FUT7 (FIG. 21A). During these steps, different amounts of enzymes, and multiple reaction times were applied to reveal the intermediate products. Indeed, in FIG. 21B, the intermediate products of G1, A1[3] and A1[6] during the synthesis of G2, A2[3], and A2[6], respectively, were observed. While it only took 10 minutes for the complete conversion of G0 to G2 by B4GalT1, it took 5 hours for the complete conversion of G2 to A2[3] by ST3Gal4 and to A2[6] by ST6Gal1. No intermediate product was initially observed during the conversion of A2[3] to A2[3]F2 by FUT7 in a 5-hour reaction. To search for the intermediate product, additional experiments were performed with FUT6 and FUT7 with only a 20-minute reaction. FIG. 21C shows the transition of A2[3] to A2[3]F2 by both FUT6 and FUT7, but with no distinct band for the intermediate, likely because the mobility shift caused by the modification is too small. Similar observation was made on the transition of G2 to G2F2 by FUT6 or FUT9 (FIG. 21D).

Intermediate products are signs for the progress of each enzymatic step. Based on the amount of enzyme and the time needed for reaching the completion of each reaction, the relative velocities and therefore the activities of the enzymes in FIG. 21 were estimated (Table 3). It is noted that the FUTs have much faster kinetics than the sialyltransferases with the difference from 1 to 3 orders of magnitude. Considering that the FUTs are responsible for the final steps of Le^{x} and sLe^{x} formation, it is likely that these enzymes are subject to strict regulation therefore allow the cells to quickly respond to biological stimulation.

TABLE 3
Relative activities of the enzymes for Le^{x} and
sLe^{x} synthesis based on reaction completion* in FIG. 6

| | | | Activity based | |
|---|---|---|---|---|
| | | | on reaction | |
| | | Completed | completion | Relative |
| Enzyme | Substrate | Product | (pmol/min/μg)* | activity** |
| FUT6 | A2[3] | sLe^{x} | 13.5 | 100 |
| FUT6 | G2 | Le^{x} | 2.3 | 17 |
| FUT7 | A2[3] | sLe^{x} | 0.38 | 2.8 |
| FUT9 | G2 | Le^{x} | 2.3 | 17 |
| ST3Gal4 | G2 | A2[3] | 0.0084 | 0.06 |
| ST6Gal1 | G2 | A2[6] | 0.025 | 0.19 |
| B4GalT1 | G0 | G2 | 2.3 | 17 |

Note:
*The reaction velocity was calculated based on the time and amount of an enzyme required for the completion of a reaction but rather than the initial velocity used in Michaelis-Menten kinetics. Activities calculated based on completed reactions should be substantially lower than initial velocities but still give good estimations of overall real activities.
**Relative activities were normalized to that of FUT6 on sLe^{x} synthesis.

### Quantification of Cy5-Labeled Glycans by Establishing a Response Curve for Labeled G0 Glycan Standard

To evaluate the quantitative aspects of the glycan electrophoresis described in this article, a series of enzymatic reactions towards Le^{a} synthesis were run along with a 2-fold serial dilution of the FUT8-labeled antibody glycan G0 (G0f) (FIG. 22). Le^{a} was synthesized from G0f sequentially via the steps of galactosylation by B3GalT2, sialylation by ST3Gal3 and fucosylation by FUT3 (FIG. 22A). Major intermediate products of Le^{a} synthesis including G1[3]f, A1G1[3]f and A1 G1[3]F1f were observed (FIG. 22B). When the band intensities of the serial dilution of G0f in FIG. 22B were plotted against the masses of the glycan, a response curve with the linear coefficient of 0.9998 was obtained, with the slope of the curve representing the signal to mass ratio (FIG. 22C). The masses of the intermediates of Le^{a} synthesis were then calculated using the signal to mass ratio. While the reactions by ST3Gal3 and FUT3 were almost completed, B3GalT2 converted 53% and 9% of G0f to G1[3]f and G2[3]f, respectively. The results indicate that fluorophore-labeled glycans separated by electrophoresis can be quantitatively measured when a standard curve is established. In addition, the results also indicate that the sensitivity of the current method for glycan detection is below picomole level.

### Discussion

The results of this Example provide further evidence that various fluorophore-conjugated fucoses are well tolerated by FUTs. By incorporating these conjugated fucoses to target glycans and separating them through gel electrophoresis, the substrate glycans as well as the substrate specificities of these FUTs were elucidated. More specifically, specific N-glycans were detected on therapeutic antibodies, RNase B and recombinant influenza viral neuraminidase. This Example also demonstrated step-by-step enzymatic synthesis of Le^{x} and sLe^{x} from defined glycan structure, and further revealed that the responsible FUTs have kinetics 1 to 3 orders of magnitude faster than those of corresponding sialyltransferases. Together with other glycosyltransferases and glycosidases, the methods allow quick detection of certain glycans and kinetic study of the biosynthesis of certain glycan epitopes. These results support the notion that FUTs are subject to strict regulation for their roles in Le^{x} and sLe^{x} biosynthesis.

### Example 4— Glycan Fingerprinting of SARS2 Spike Proteins

This Example describes a method of glycan fingerprinting based on enzymatic fluorescent labeling and gel electrophoresis. The method is illustrated on SARS-2 spike (S) glycoproteins. The SARS-2 coronavirus (causative agent of COVID-19 pandemic) uses the extensively glycosylated S protein to mediate its infection process. Although the S protein is the principal target of many vaccines in development, glycosylation of the S protein, due to its complexity and variability, presents a major challenge for generating an effective vaccine.

As further described in this Example, to obtain the glycan fingerprint of a S protein, glycans released from the protein were first labeled through enzymatic incorporation of fluorophore-conjugated sialic acid or fucose, then separated on acrylamide gel through electrophoresis, and finally visualized with a fluorescent imager. To identify the labeled glycans of a fingerprint, glycan standards and glycan ladders that were enzymatically generated were run alongside the samples as references. By comparing the mobility of a labeled glycan to that of a glycan standard and the mobility shifts caused by additional enzymatic modification, the identity of glycans may be determined.

### Material and Methods

### Protein Sources

Recombinant SARS-CoV-2 Spike RBD proteins expressed in HEK293 cells, Tn5 insect cells, CHO cells; full length recombinant SARS-CoV-2 Spike proteins expressed in HEK293 cells and CHO cells; and recombinant SARS-CoV-2 Spike 51 subunit protein expressed in HEK293 cells were from Bio-Techne (Minneapolis, MN). Recombinant human ST6Gal1, FUT8, B4GalT1, MGAT1, ST3Gal6, FUT9, ST3Gal4 and ST3Gal3, and C. perfringens neuraminidase and F. meningosepticum PNGase, CMP-Cy5-Siallic acid, GDP-AlexaFluor555-Fucose were from Bio-Techne (Minneapolis, MN). IgG glycan G0, G1F and G0F were from Dextra Laboratories (Reading, United Kingdom).

### Releasing and Labeling of the Glycans of Spike Proteins

To release N-glycans, 5 μg of a spike protein was mixed with 0.2 μg PNGase F and supplemented with labeling buffer (25 mM Tris pH 7.5, 10 mM MnCl₂) to 20 μL and then incubated at 37° C. for 30 minutes. For desialylation, an additional 0.2 μg C.p. neuraminidase was also added into the reaction mixture. The above mixture was then heated at 95° C. for two minutes to inactivate the enzymes. Labeling mixture contained 0.5 μg of a sialyltransferase together with 0.4 nmol of CMP-Cy5-Sialic Acid supplemented with labeling buffer to 10 μl. In the case for labeling oligomannose, additional 0.5 μg of FUT8 together with 0.4 nmol of GDP-AlexaFluor555-Fuc and 0.5 μg of MGAT1 together with 10 nmol of UDP-GlcNAc were also added into the labeling mixture. The labeling mixture was then added into the reaction mixture and incubate at 37° C. for 1 to 2 hours or overnight at room temperature.

### Labeling Glycan Standards and Building Glycan Ladder

For labeling a glycan with Cy5-Sialic Acid, 1 μg of the standard was mixed with 1 μg of ST6Gal1 and 1 nmol of CMP-Cy5-Sialic Acid together with 0.5 μg B4GalT1 and 10 nmol of UDP-Gal supplemented with labeling buffer to 20 μL and the mixture was incubated at 37° C. for 2 hours or overnight at room temperature. For labeling a glycan standard with Cy5-Fucose, 2 μg of the standard was mixed with 1 μg of FUT8 and 2 nmol of GDP-Cy5-Fucose supplemented with labeling buffer to 20 μL and the mixture was incubated at 37° C. for 2 hours or overnight at room temperature. For building a glycan ladder based on Cy5-Fucose labeled glycan standard, 200 ng of the above labeled glycan was extended with additional one or more of 0.5 μg each of the glycosyltransferases including MGAT3, MGAT5, B4GalT1, FUT9, ST3Gal6 and ST6Gal1 together with their donor substrates at 37° C. for 2 hours or overnight at room temperature or whenever the reactions were completed. The reactions were then stopped by heating at 95° C. for 2 minutes. Glycan ladder was built by mixing equal amounts of the extended labeled glycans described above.

### Glycan Electrophoresis and Imaging

All labeled samples including, for example, glycan standards were separated by sodium dodecyl sulfate—polyacrylamide gel electrophoresis (SDS-PAGE) in 15% or 17% SDS gels at 20 volts/cm. After separation, gels were imaged using a FluorChem M imager (ProteinSimple, Bio-Techne, Minneapolis, MN). To image protein content, the gels were also imaged with traditional methods such as silver staining or trichloroethanol (TCE) staining.

### Results

### Non-Reducing End Based Naming of N-Glycans

As described in Example 3, a fluorophore-conjugated glycan may be separated by SDS-PAGE. To apply the method to glycan analysis, it is preferred to identify those separated glycans.

The nomenclature of these N-glycans for the purpose of this Example is described in FIG. 23 and below. Only monosaccharides at the non-reducing ends of an N-glycan are specified. The rules of this method are the following. First, sialic acid, galactose, mannose, glucosamine and fucose that are commonly found at the non-reducing ends of N-glycans are represented with capital letter S, G, M, N, and F, respectively, with the exception that bisecting GlcNAc and core fucose are represented with small letter n and f, respectively. Second, a prime symbol is used to indicate if a monosaccharide is conjugated to a fluorophore. Third, the number of a non-reducing end monosaccharide of an N-glycan is specified after the letters, which is usually no more than five as this is the maximal number of the branches found on an N-glycan. Fourth, the linkage of a non-reducing end monosaccharide is represented as a number within square brackets ([ ]). For example, S1[3]N1nf′ represents a biantennary N-glycan that contains an α,3-linked sialic acid, a GlcNAc residue with no specification on linkage, a bisecting GlcNAc and a fluorophore-conjugated core fucose at its non-reducing ends. Since there is only one bisecting GlcNAc residue and only one core fucose residue on common N-glycans, the number and linkage for these monosaccharides are omitted.

### Electrophoretic Mobility of Cy5-Labeled Glycans on SDS PAGE

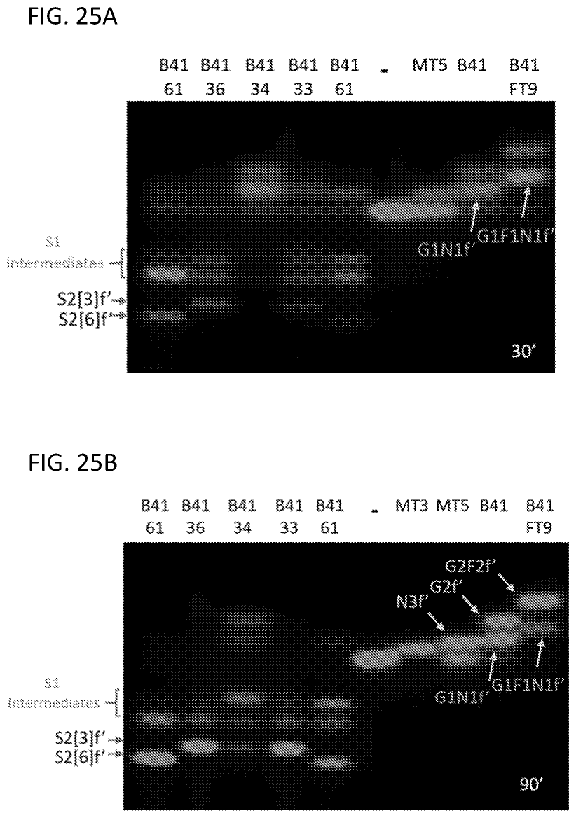

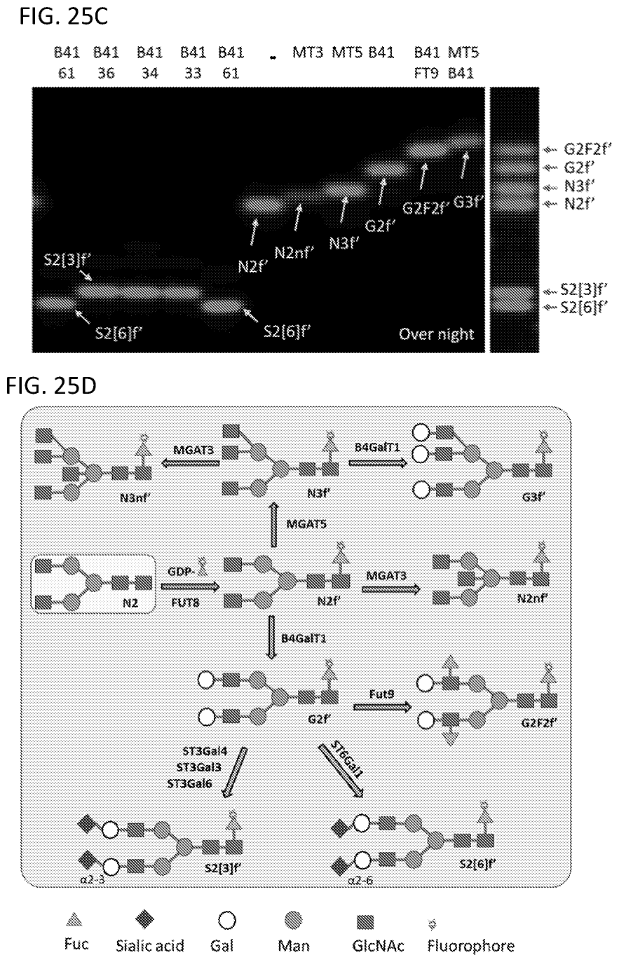

To correlate the glycan structures to their mobility, a series of labeled glycans based on a biantennary antibody glycan N2 (known as G0 in common antibody glycan nomenclature) were established and separated using SDS-PAGE (FIG. 24). N2 was first labeled by FUT8 with Cy5-conjugated fucose to become N2f′. A series of glycans were then generated enzymatically based on N2f′ (FIG. 24A and FIG. 24B). N2 was also extended by B4GalT1 with or without prior modification by FUT8 and finally labeled by ST6Gal1 with Cy5-conjugated sialic acid to generate S′1[6]G1f and S′1[6]G1 (FIG. 24C). The following observations were made regarding the mobility change caused by the addition of different monosaccharides. First, addition of a neutral monosaccharide such as a Gal, GlcNAc, and Fuc to a glycan slows down the mobility of the glycan at a noticeable rate. Second, addition of a bisecting GlcNAc slows down the mobility of a glycan at roughly the half rate of that of a β,6-linked GlcNAc. Third, addition of a sialic acid residue significantly increases the mobility of a glycan, with even more increase on mobility by an α,6-linked sialic acid than by an α,3-linked sialic acid. Likewise, when a monosaccharide can be added at multiple positions on a glycan, intermediate glycosylation products exhibit intermediate mobilities, for example, G1N1f′ moves faster than G2f′, and S1[6]G1f′ moves slower than S2[6]f′ (FIG. 25). Intermediate products were only observed within a short time window and were converted to final products after prolonged incubation.

### Selection of Labeling Enzyme and Optimization of Substrate Concentrations

Before fingerprinting glycans released from various SARS2 spike proteins, the labeling enzymes were screened and the substrate concentration for the labeling reaction was optimized using glycans released from the RBD protein expressed in CHO cells as the substrates. The glycans were first probed by various sialyltransferases, including ST6Gal1 that generates α2,6-sialylated N-glycans (Weinstein et al. J Biol Chem 262, 17735-17743 (1987)), and, ST3Gal3, ST3Gal4 and ST3Gal6 that generate α2,3-sialylated N-glycans (Qi et al. FASEB J 34, 881-897 (2020), Okajima et al. J Biol Chem 274, 11479-11486 (1999)). Among these enzymes, ST6Gal1 and ST3Gal6 gave stronger signals (FIG. 26A) and were chosen for the following glycan fingerprinting study. Stronger signal intensities were also observed when the substrate input was around 2 μg (FIG. 26B) and the donor CMP-Cy5-Sialic Acid input was around 0.4 nmol (FIG. 26C), therefore these conditions were chosen for the following fingerprinting study.

Glycan Finger-Printing Study of SARS-2 Spike Proteins with ST6Gal1

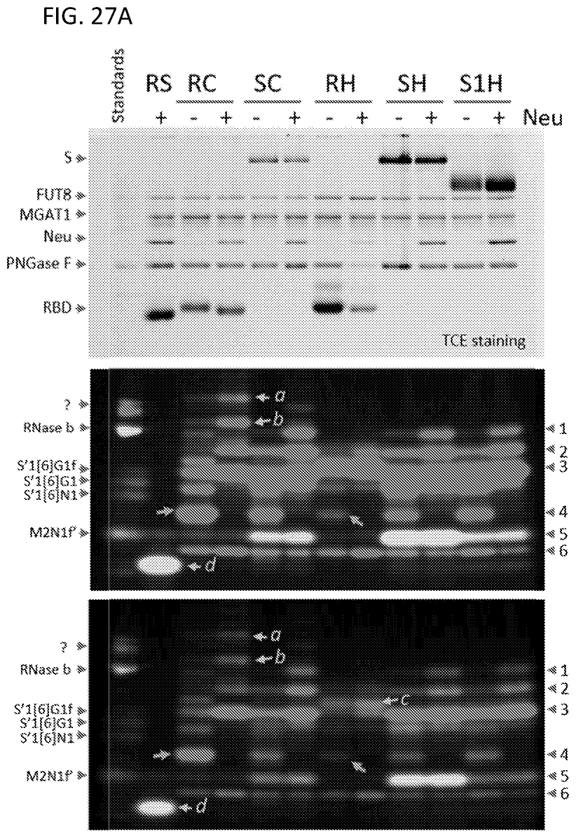

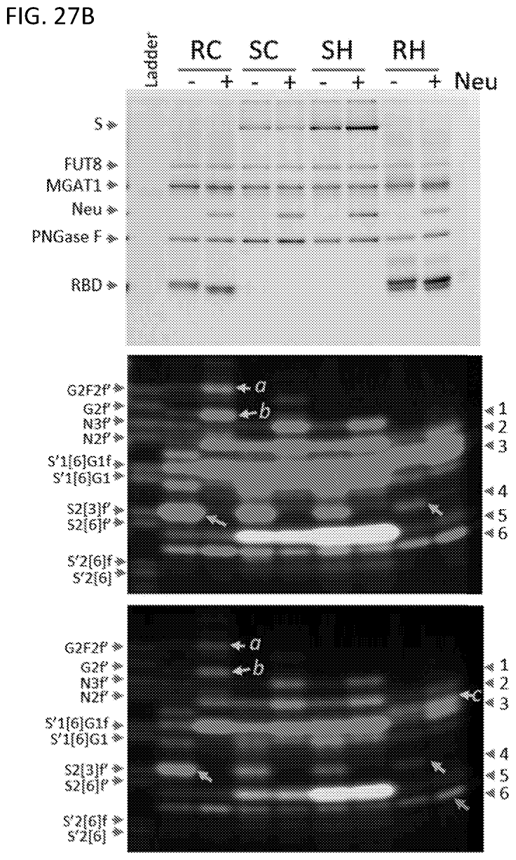

N-glycans released from the following SARS-2 spike protein constructs with or without prior desialylation were then labeled with ST6Gal1/CMP-Cy5-Sialic Acid: RBD domain expressed in Sf21 cells (RS), RBD domain expressed in CHO cells (RC), RBD domain expressed in HEK293 cells (RH), full length spike protein expressed in CHO cells (SC), full length spike protein expressed in HEK293 cells (SH), and 51 protein expressed in HEK293 cells (S1H). As the presence of oligomannose glycans on S proteins were reported previously (Shajahan et al. Glycobiology, cwaa042 (2020), Watanabe et al. Science 369, 330-333 (2020)), FUT8/GDP-AlexaFluor555-Fuc together with MGAT1/UDP-GlcNAc that allows the labeling of Man3 and Man5 (Wu et al. Glycobiology, cwaa030 (2020)) were also added into the final labeling reactions to reveal these glycans. ST6Gal1 labeling revealed a series of bands with large variations from all constructs except RS (FIG. 27A). In general, desialylation resulted in elimination of some fast-moving bands and increased labeling on some slow-moving bands, suggesting the existence of both sialylated and asialylated glycans on these proteins.

Several common bands were observed (e.g., bands 1 to 6 in FIG. 27). Bands 1 and 2 were mainly found in neuraminidase treated samples of SC, SH and S1H at the position around N3f′ and N₂f (FIG. 27B). Since labeling through ST6Gal1 also contributes a sialic acid and therefore makes a labeled glycan move much faster, band 1 and band 2 could result from labeling of highly branched complexed glycans, such as tetra- and tri-antennary complex glycans. Band 1 was mainly observed in desialylated SC, SH and S1H, suggesting that the glycan was initially sialylated. Band 2 was observed in SC, SH and S1H samples before and after desialylation, but with great signal increase upon desialylation, suggesting that the glycan was initially largely sialylated. Band 3 was prominent in all SH and S1H samples and in desialylated samples of RC and SC and had the same mobility of S′1[6]G1f (FIG. 27B). Since band 3 and the reference glycan S′1[6]G1f had the same labeling (both labeled on α,6-linked sialic acid with Cy5) and same mobility, band 3 likely had the same structure as S′1[6]G1f and was the labeling product of G2f (FIG. 24E). The fact that band 3 was much weaker in RC and SC than in desialylated RC and SC samples suggests that the glycan was initially sialylated in these samples. Opposite to band 3, band 4 around the position of S2[3]f exhibited a strong presence in RC and SC but not in the desialylated RC and SC samples, suggesting that band 4 was a result of the labeling of a partially sialylated glycan that was converted to band 3 when desialyation occurred before labeling. Band 5 was likely due to the labeling of oligomannose M3 (known as Man5) because the band had same mobility of the reference glycan M2N1f′ (FIG. 27A). Band 6 had almost equal intensity in all samples and did not respond to C.p Neuraminidase treatment. The fast mobility of band 6 suggests that it is highly sialylated, but its unresponsiveness to neuraminidase treatment suggests the opposite. The nature of band 6 remains to be investigated.

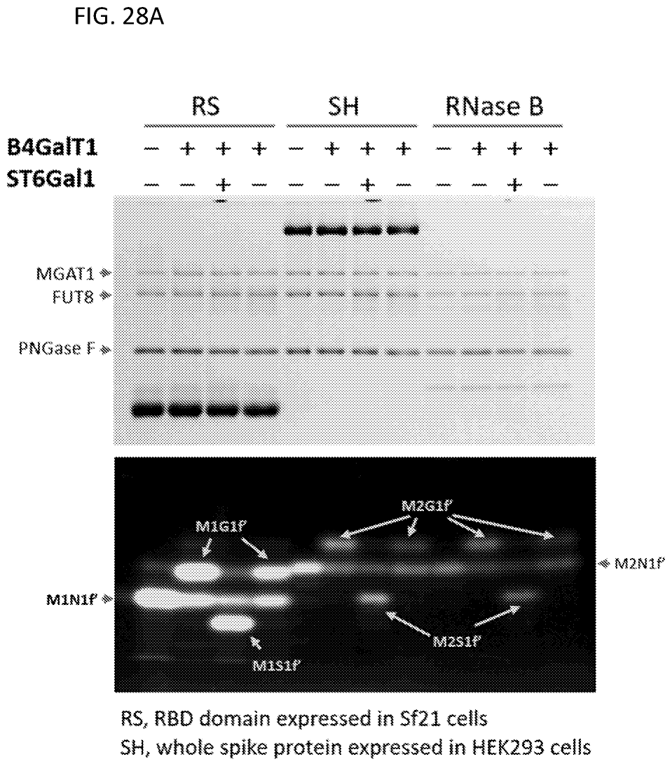

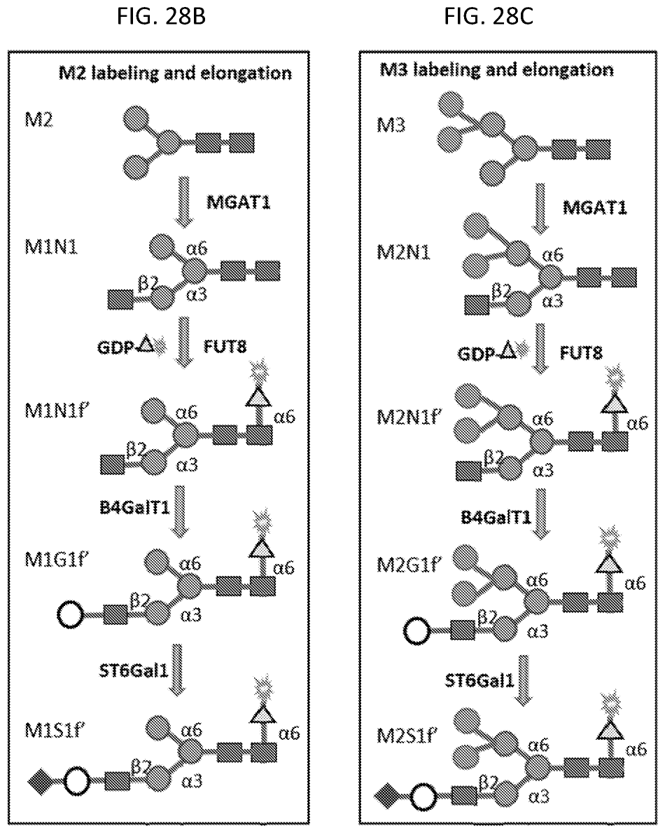

Most of the common bands displayed great variation among the samples. For example, band 4 was the most abundant in RC but almost at negligible level in RH (blue arrows in FIG. 27); band 5 was the most abundant in SH but almost completely lacking in RH. Surprisingly, some bands were only found in one sample but not the others, such as band a, b, c and d (FIG. 27A). Band a and b in RC had slow mobility and responded to neuraminidase treatment, suggesting that they had highly complexed structures and were initially sialylated. Band c in RH was just above the position of S′1[6]G1f, suggesting that it might be S′1[6]G1fn that contains a bisecting GlcNAc (FIG. 24B and FIG. 24C). Band d labeled by FUT8 was found only in RS and had faster mobility than M2N1f′, suggesting that it be M1N1f′ (labeled product of Man3), in consistent with the notion that Man3 is a main glycan expressed in insect cells (Shi et al. Curr Drug Targets 8, 1116-1125 (2007)). Additional enzymatic conversion of band d with B4GalT1 and ST6Gal1 further confirmed the identity of band d (FIG. 28).

Glycan Fingerprinting Study of SARS-2 Spike Proteins with ST3Gal6

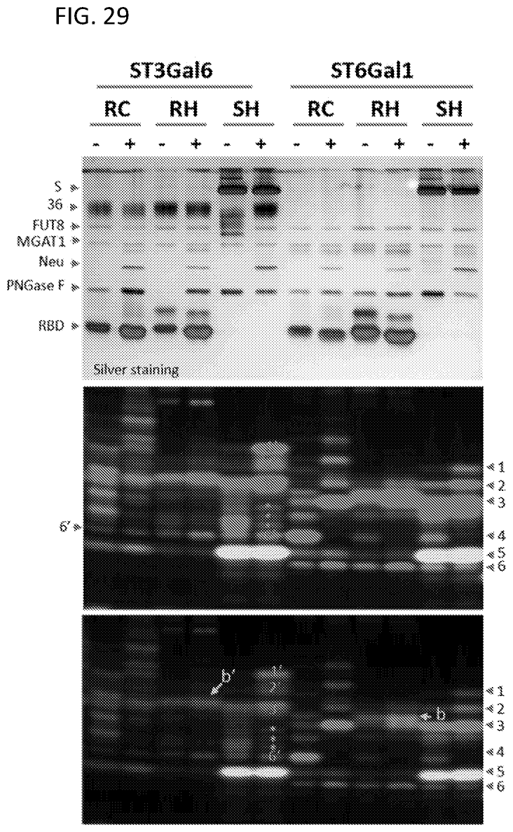

Both ST6Gal1 and ST3Gal6 are known to sialylate the Galβ1,4 GlcNAc structure on glycoproteins (Okajima et al. J Biol Chem 274, 11479-11486 (1999)). When the same set of the SARS-2 spike protein samples were probed with ST3Gal6, similar but distinctive glycan fingerprints were observed (FIG. 29). It seems that the entire band pattern revealed by ST3Gal6 was upshifted from that of ST6Gal 1. For example, the bands 1′, 2′, 3′, and 6′ in THE ST3Gal6-labeled SH sample corresponded well with the bands 1, 2, 3, and 6 in ST6Gal1-labeled SH sample; similar to band 6, band 6′ was found across all lanes; similar to the relative positioning of band b and band 3 revealed by ST6Gal1, band b′ labeled by ST3Gal6 was slightly upshifted from band 3′. The upshift observed of the bands revealed by ST3Gal6 from those by ST6Gal1 is likely a result of the slower mobility of glycans with α,3-linked sialic acid compared to corresponding glycans with α,6-linked sialic acid (FIG. 24).

While the glycan fingerprints revealed by the two enzymes were similar, ST3Gal6 labeling also revealed some unique bands. For example, bands marked with asterisks in SH revealed by ST3Gal6 had no corresponding bands in SH revealed by ST6Gal1 (FIG. 29). These differences suggest that ST3Gal6 and ST6Gal1 have overlapping but distinctive substrate preferences.

### Discussion

The data presented in this Example suggest that the RBD of the SARS2 spike protein expressed in HEK293 cells mainly contains complex glycans, consistent with the reports of Watanabe, et al (Watanabe et al. Biochim Biophys Acta Gen Subj 1863, 1480-1497 (2019)). These data also suggest that bisecting GlcNAc may mainly exist on the RBD portion, and oligomannose glycans may mainly exist on other parts of S protein except the RBD portion when expressed in HEK293 cells. These data further indicate that the glycans of S proteins expressed in insect cells and HEK293 cells are completely different. Altogether, the following may be concluded for the glycosylation of the SARS2 spike proteins: the type of host cell determines the types of glycans attached to the spike proteins; protein primary sequence determines if the protein is glycosylated; and secondary and tertiary structure may affect the type of glycosylation as well.

The complete disclosure of all patents, patent applications, and publications, and electronically available material (including, for instance, nucleotide sequence submissions in, for example, GenBank and Ref Seq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A composition comprising a fluorophore-conjugated sialic acid or a fluorophore-conjugated fucose or both a fluorophore-conjugated sialic acid and a fluorophore-conjugated fucose.

2. The composition of claim 1,
wherein the fluorophore-conjugated sialic acid comprises an activated fluorophore-conjugated sialic acid, or
wherein the fluorophore-conjugated fucose comprises an activated fluorophore-conjugated fucose, or
both.

3. The composition of claim 2,
wherein the fluorophore-conjugated sialic acid comprises a cytidine monophosphate activated fluorophore-conjugated sialic acid (CMP-f-SA), or
wherein the fluorophore-conjugated fucose comprises guanosine diphosphate activated fluorophore-conjugated fucose (GDP-f-Fuc), or
both.

4. The composition of claim 1, wherein the fluorophore-conjugated sialic acid comprises N-acetyl-neuraminic acid (Neu5Ac or NANA), 2-keto-3-deoxynononic acid (Kdn), N-glycolylneuraminic acid (Neu5Gc), neuraminic acid (Neu), or 2-deoxy-2,3-didehydro-Neu5Ac (Neu2en5Ac), or a combination thereof.

5. The composition of claim 1,
wherein the fluorophore of the fluorophore-conjugated sialic acid comprises Alexa Fluor® 488, Alexa Fluor® 555, or Cy5; or
wherein the fluorophore of the fluorophore-conjugated fucose comprises Alexa Fluor® 488, Alexa Fluor® 555, or Cy5.

6. A method comprising making a composition comprising a fluorophore-conjugated sialic acid or a fluorophore-conjugated fucose or both, wherein the method comprises
incubating a CMP-Azido-Sialic acid (CMP-N₃-SA) and an alkyne-conjugated fluorophore, or
incubating a GDP-Azido-Fucose (GDP-N₃-Fucose) and an alkyne-conjugated fluorophore.

7. The method of claim 6, wherein the CMP-N₃-SA and the alkyne-conjugated fluorophore or the GDP-N₃-Fucose and the alkyne-conjugated fluorophore are conjugated via copper (1)-catalyzed azide-alkyne cycloaddition.

8. The method of claim 6, wherein the method further comprises
forming cytidine monophosphate activated fluorophore-conjugated sialic acid (CMP-f-SA), or
forming guanosine diphosphate activated fluorophore-conjugated fucose (GDP-f-Fuc).

9. The method of claim 8, wherein the method further comprises:
purifying the CMP-f-SA or the GDP-f-Fuc; and/or
concentrating the CMP-f-SA or the GDP-f-Fuc.

10. (canceled)

11. A method comprising:
attaching the fluorophore-conjugated sialic acid of the composition of claim 1 to a glycan, or
attaching the fluorophore-conjugated fucose of the composition of claim 1 to a glycan, or
attaching both the fluorophore-conjugated sialic acid and the fluorophore-conjugated fucose to a glycan.

12. The method of claim 11, wherein the method comprises
attaching the fluorophore-conjugated sialic acid to a glycan using a sialyltransferase, or
attaching the fluorophore-conjugated fucose to a glycan using a fucosyltransferase.

13. The method of claim 12,
wherein the sialyltransferase comprises ST3Gal1, ST3Gal2, ST3Gal3, ST3Gal4, ST3Gal5, ST3Gal6, ST6Gal 1, ST6Gal2, ST6GalNAc1, ST6GalNAc2, ST6GalNAc3, ST6GalNAc4, ST6GalNAc5, ST6GalNAc6, ST8SIA1, ST8SIA2, ST8SIA3, ST8SIA4, ST8SIA5, or ST8SIA6, or a combination thereof;
wherein the fucosyltransferase comprises FUT1, FUT2, FUT3, FUT4, FUT5, FUT6, FUT7, FUT8, FUT9, FUT10, or FUT11, or a combination thereof; or
both.

14. The method of claim 11, wherein the method comprises mixing
the glycan or a target protein comprising the glycan,
a fluorophore-conjugated sugar comprising the fluorophore-conjugated sialic acid or the fluorophore-conjugated fucose or both, and
an enzyme comprising a sialyltransferase or a fucosyltransferase or both,
wherein the fluorophore-conjugated sugar is attached to the glycan to form a labeled glycan or a labeled target protein.

15. The method of claim 14, wherein the method comprises
mixing the glycan or target protein, the fluorophore-conjugated sugar, and the enzyme in a buffer,
incubating the glycan or target protein, the fluorophore-conjugated sugar, and the enzyme in a buffer together for at least 1 minute and up to 48 hours, and/or
incubating the glycan or target protein, the fluorophore-conjugated sugar, and the enzyme at a temperature of at least 20° C. and up to 50° C.

16. The method of claim 14, wherein the method comprises mixing the glycan or target protein, the fluorophore-conjugated sugar, and the enzyme with
a neuraminidase, a galactosidase, α-2 mannosidase, or MGAT1, or a combination thereof.

17. The method of claim 14, wherein the method further comprises separating components of a mixture comprising the labeled glycan or separating components of a mixture comprising the labeled target protein.

18. The method of claim 17, wherein the method further comprises:
separating components of the mixture comprising the labeled glycan or separating components of the mixture comprising the labeled target protein comprises gel electrophoresis and/or
imaging the labeled glycan or labeled target protein.

19. (canceled)

20. The method of claim 14, wherein the method comprises cleaving the labeled glycan from the labeled target protein to form a freed labeled glycan.

21. The method of claim 20, wherein the method comprises comparing mobility of the freed labeled glycan to mobility of a glycan standard or a glycan ladder, wherein the glycan standard comprises a fluorophore-conjugated glycan and wherein the glycan ladder comprises two or more fluorophore-conjugated glycans.

22. (canceled)

23. A composition comprising a glycan ladder, wherein the glycan ladder comprises at least two fluorophore-conjugated glycans.
